(19) United States
(12) Patent Application Publication
(10) Pub. No.: US 2025/0257059 A1
(43) Pub. Date: Aug. 14, 2025

(54) PROTEIN TYROSINE PHOSPHATASE INHIBITORS AND USES THEREOF

(71) Applicant: Nerio Therapeutics, Inc., La Jolla, CA (US)

(72) Inventors: Jason ROLAND, San Diego, CA (US); Sergio G. DURON, San Diego, CA (US); Christian PEREZ, Chula Vista, CA (US)

(21) Appl. No.: 18/855,786

(22) PCT Filed: Apr. 13, 2023
(86) PCT No.: PCT/US2023/018508

§ 371 Date: Oct. 10, 2024

Related U.S. Application Data
(60) Provisional application No. 63/486,559, filed on Feb. 23, 2023.
(60) Provisional application No. 63/331,073, filed on Apr. 14, 2022.

(51) Int. Cl. C07D 417/10; A61K 31/433; A61K 31/438; A61K 31/439; A61K 31/454; A61K 31/4545; A61K 31/496; A61K 31/5377; A61K 31/55; A61P 35/00; C07D 285/10; C07D 417/14; C07D 471/08; C07D 491/107

(52) U.S. Cl. CPC C07D 417/10; A61K 31/433; A61K 31/438; A61K 31/439; A61K 31/454; A61K 31/4545; A61K 31/496; A61K 31/5377; A61K 31/55; A61P 35/00; C07D 285/10; C07D 417/14; C07D 471/08; C07D 491/107

(57) ABSTRACT

Provided herein are compounds, compositions, and methods useful for inhibiting protein tyrosine phosphatase, e.g., protein tyrosine phosphatase non-receptor type 2 (PTPN2) and/or protein tyrosine phosphatase non-receptor type 1 (PTPN1), and for treating related diseases, disorders, and conditions favorably responsive to PTPN1 or PTPN2 inhibitor treatment, e.g., a cancer or a metabolic disease.

### CROSS-REFERENCE

This application claims the benefit of U.S. Provisional Application Ser. No. 63/331,073 filed Apr. 14, 2022 and U.S. Provisional Application Ser. No. 63/486,559 filed Feb. 23, 2023; which are hereby incorporated by reference in their entirety.

### BACKGROUND OF THE INVENTION

Cancer immunotherapy regimens targeting immune evasion mechanisms including checkpoint blockade (e.g., PD-1/PD-L1 and CTLA-4 blocking antibodies) have been shown to be effective in treating in a variety of cancers, dramatically improving outcomes in some populations refractory to conventional therapies. However, incomplete clinical responses and the development of intrinsic or acquired resistance continue to limit the patient populations who could benefit from checkpoint blockade.

Protein tyrosine phosphatase non-receptor type 2 (PTPN2), also known as T cell protein tyrosine phosphatase (TC-PTP), is an intracellular member of the class I subfamily of phospho-tyrosine specific phosphatases that control multiple cellular regulatory processes by removing phosphate groups from tyrosine substrates. PTPN2 is ubiquitously expressed, but expression is highest in hematopoietic and placental cells. In humans, PTPN2 expression is controlled post-transcriptionally by the existence of two splice variants, a 45 kDa form that contains a nuclear localization signal at the C-terminus upstream of the splice junction, and a 48 kDa canonical form which has a C-terminal ER retention motif. The 45 kDa isoform can passively transfuse into the cytosol under certain cellular stress conditions. Both isoforms share an N-terminal phospho-tyrosine phosphatase catalytic domain. PTPN2 negatively regulates signaling of non-receptor tyrosine kinases (e.g., JAK1, JAK3), receptor tyrosine kinases (e.g., INSR, EGFR, CSF1R, PDGFR), transcription factors (e.g., STAT1, STAT3, STAT5a/b), and Src family kinases (e.g., Fyn, Lck). As a critical negative regulator of the JAK-STAT pathway, PTPN2 functions to directly regulate signaling through cytokine receptors, including IFNγ. The PTPN2 catalytic domain shares 74% sequence homology with PTPN1 (also called PTP1B), and shares similar enzymatic kinetics. Data from a loss of function in vivo genetic screen using CRISPR/Cas9 genome editing in a mouse B16F10 transplantable tumor model show that deletion of Ptpn2 gene in tumor cells improved response to the immunotherapy regimen of a GM-CSF secreting vaccine (GVAX) plus PD-1 checkpoint blockade. Loss of PTPN2 sensitized tumors to immunotherapy by enhancing IFNγ-mediated effects on antigen presentation and growth suppression. The same screen also revealed that genes known to be involved in immune evasion, including PD-L1 and CD47, were also depleted under immunotherapy selective pressure, while genes involved in the IFNγ signaling pathway, including IFNGR, JAK1, and STAT1, were enriched. These observations point to a putative role for therapeutic strategies that enhance IFNγ sensing and signaling in enhancing the efficacy of cancer immunotherapy regimens.

Protein tyrosine phosphatase non-receptor type 1 (PTPN1), also known as protein tyrosine phosphatase-1B (PTP1B), has been shown to play a key role in insulin and leptin signaling and is a primary mechanism for down-regulating both the insulin and leptin receptor signaling pathways. Animals deficient in PTP1B have improved glucose regulation and lipid profiles and are resistant to weight gain when treated with a high fat diet. Thus, PTP1B inhibitors are expected to be useful for the treatment of type 2 diabetes, obesity, and metabolic syndrome.

### SUMMARY OF THE INVENTION

Disclosed herein is a compound of Formula (I), or a pharmaceutically acceptable salt, solvate, or stereoisomer thereof:

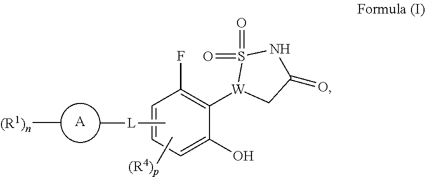

wherein:
Ring A is a heterocycloalkyl or heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R^{1a};
or two R¹ on the same atom are taken together to form an oxo;
or two R¹ on the same carbon are taken together to form a cycloalkyl or heterocycloalkyl; each optionally substituted with one or more R;
or two R¹ on the different atoms are taken together to form a cycloalkyl, heterocycloalkyl, aryl, or heteroaryl; each optionally substituted with one or more R;
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{a}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R;
or two R^{1a} on the same atom are taken together to form an oxo;
n is 0-11;
L is —O—, —S—, —S(═O)—, —S(═O)₂—, —NR²—, [C(R³)₂]ₘ—, —O[C(R³)₂]ₘ—, —NR²[C(R³)₂]ₘ, —[C(R³)₂]ₘO—, or —[C(R³)₂]ₘNR²—;
R² is hydrogen, —C(═O)R^{a}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
each R³ is independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R;
or two R³ are taken together to form a cycloalkyl or heterocycloalkyl; each optionally substituted with one or more R;
m is 1-4;
each R⁴ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
p is 0-2;
W is CR^{W} or N;
R^{W} is hydrogen, deuterium, halogen, —CN, —NO₂, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{a}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently and optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently and optionally substituted with one or more R;
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently and optionally substituted with one or more R;
or R^{c} and R^{d} are taken together with the atom to which they are attached to form a heterocycloalkyl optionally substituted with one or more R; and
each R is independently halogen, —CN, —OH, —OC₁-C₃alkyl, —OC₁-C₃haloalkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃deuteroalkyl, C₁-C₃haloalkyl, C₁-C₃hydroxyalkyl, C₁-C₃aminoalkyl, C₁-C₃heteroalkyl, or C₃-C₆cycloalkyl;
or two R on the same atom form an oxo.

In some embodiments of a compound of Formula (I), the compound is of Formula (Ia):

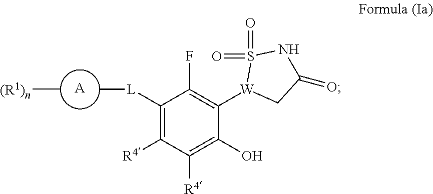

wherein R^{4′} is hydrogen or R⁴.

In some embodiments of a compound of Formula (I), the compound is of Formula (Ib):

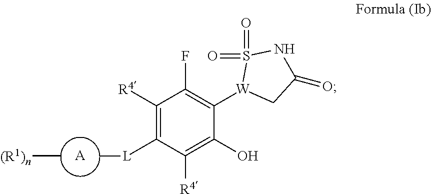

wherein R^{4′} is hydrogen or R⁴.

In some embodiments of a compound of Formula (I), the compound is of Formula (Ic):

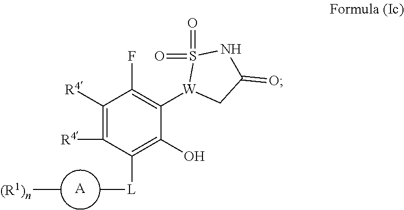

wherein R^{4′} is hydrogen or R⁴.

Also disclosed herein is a compound of Formula (II), or a pharmaceutically acceptable salt, solvate, or stereoisomer thereof:

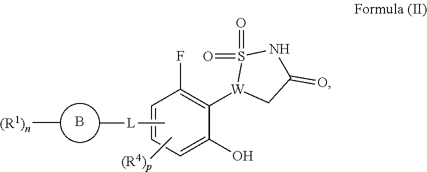

wherein:
Ring B is a cycloalkyl, heterocycloalkyl, or heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{e}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R^{1a};
or two R¹ on the same atom are taken together to form an oxo;
or two R¹ on the same carbon are taken together to form a cycloalkyl or heterocycloalkyl; each optionally substituted with one or more R;
or two R¹ on the different atoms are taken together to form a cycloalkyl, heterocycloalkyl, aryl, or heteroaryl; each optionally substituted with one or more R;
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{a}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R;
or two R^{1a} on the same atom are taken together to form an oxo;
n is 1-11;
L is —O—, —S—, —S(═O)—, —S(═O)₂—, —NR²—, [C(R³)₂]ₘ—, —NR²S(═O)₂—, —S(═O)₂NR²—, —NR²C(═O)—, —C(═O)NR²—, —O[C(R³)₂]ₘ—, —NR²[C(R³)₂]ₘ—, [C(R³)₂]ₘ—, or [C(R³)₂]ₘNR²—;
R² is hydrogen, —C(═O)W, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
each R³ is independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R;
or two R³ are taken together to form a cycloalkyl or heterocycloalkyl; each optionally substituted with one or more R;
m is 1-4;
each R⁴ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
p is 0-2;
W is CR^{W} or N;
R^{W} is hydrogen, deuterium, halogen, —CN, —NO₂, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{a}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently and optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently and optionally substituted with one or more R;
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently and optionally substituted with one or more R;
or R^{c} and R^{d} are taken together with the atom to which they are attached to form a heterocycloalkyl optionally substituted with one or more R; and
each R is independently halogen, —CN, —OH, —OC₁-C₃alkyl, —OC₁-C₃haloalkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃deuteroalkyl, C₁-C₃haloalkyl, C₁-C₃hydroxyalkyl, C₁-C₃aminoalkyl, C₁-C₃heteroalkyl, or C₃-C₆cycloalkyl;
or two R on the same atom form an oxo.

In some embodiments of a compound of Formula (II), the compound is of Formula (IIa):

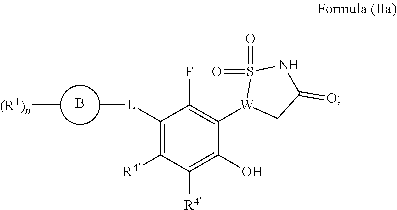

wherein R^{4′} is hydrogen or R⁴.

In some embodiments of a compound of Formula (II), the compound is of Formula (IIb):

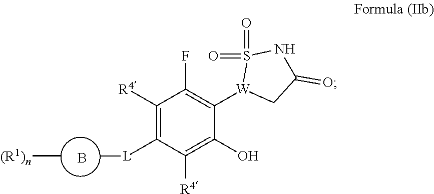

wherein R^{4′} is hydrogen or R⁴.

In some embodiments of a compound of Formula (II), the compound is of Formula (IIc):

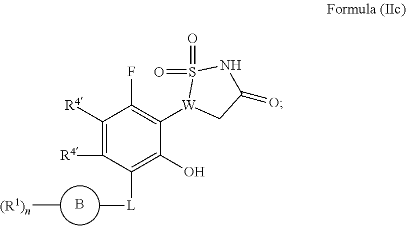

wherein R^{4′} is hydrogen or R⁴.

Also disclosed herein is a pharmaceutical composition comprising a compound disclosed herein, or a pharmaceutically acceptable salt, solvate, or stereoisomer thereof, and a pharmaceutically acceptable excipient.

Also disclosed herein is a method of treating cancer in a subject in need thereof, the method comprising administering to the subject an effective amount of a compound disclosed herein, or a pharmaceutically acceptable salt, solvate, or stereoisomer thereof.

Also disclosed herein is a method of treating cancer in a subject in need thereof, the method comprising administering to the subject a pharmaceutical composition disclosed herein.

In some embodiments, the method further comprises administering an additional therapeutic agent. In some embodiments, the additional therapeutic agent is an immunotherapeutic agent. In some embodiments, the immunotherapeutic agent is an anti-PD-1 antibody, an anti-PD-L1 antibody, or an anti-CTLA-4 antibody.

Also disclosed herein is a method of treating type-2 diabetes in a subject in need thereof, the method comprising administering to the subject an effective amount of a compound disclosed herein, or a pharmaceutically acceptable salt, solvate, or stereoisomer thereof.

Also disclosed herein is a method of treating type-2 diabetes in a subject in need thereof, the method comprising administering to the subject a pharmaceutical composition disclosed herein.

Also disclosed herein is a method of treating and/or controlling obesity in a subject in need thereof, the method comprising administering to the subject an effective amount of a compound disclosed herein, or a pharmaceutically acceptable salt, solvate, or stereoisomer thereof.

Also disclosed herein is a method of treating and/or controlling obesity in a subject in need thereof, the method comprising administering to the subject a pharmaceutical composition disclosed herein.

Also disclosed herein is a method of treating a metabolic disease in a subject in need thereof, the method comprising administering to the subject an effective amount of a compound disclosed herein, or a pharmaceutically acceptable salt, solvate, or stereoisomer thereof.

Also disclosed herein is a method of treating a metabolic disease in a subject in need thereof, the method comprising administering to the subject a pharmaceutical composition disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom) or bridged ring systems. In some embodiments, the cycloalkyl is fully saturated. Representative cycloalkyls include, but are not limited to, cycloalkyls having from three to fifteen carbon atoms (C₃-C₁₅ cycloalkyl or C₃-C₁₅ cycloalkenyl), from three to ten carbon atoms (C₃-C₁₀ cycloalkyl or C₃-C₁₀ cycloalkenyl), from three to eight carbon atoms (C₃-C₈ cycloalkyl or C₃-C₈ cycloalkenyl), from three to six carbon atoms (C₃-C₆ cycloalkyl or C₃-C₆ cycloalkenyl), from three to five carbon atoms (C₃-C₅ cycloalkyl or C₃-C₅ cycloalkenyl), or three to four carbon atoms (C₃-C₄ cycloalkyl or C₃-C₄ cycloalkenyl). In some embodiments, the cycloalkyl is a 3- to 10-membered cycloalkyl or a 3- to 10-membered cycloalkenyl. In some embodiments, the cycloalkyl is a 3- to 6-membered cycloalkyl or a 3- to 6-membered cycloalkenyl. In some embodiments, the cycloalkyl is a 5- to 6-membered cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Deuteroalkyl” refers to an alkyl radical, as defined above, that is substituted by one or more deuteriums. In some embodiments, the alkyl is substituted with one deuterium. In some embodiments, the alkyl is substituted with one, two, or three deuteriums. In some embodiments, the alkyl is substituted with one, two, three, four, five, or six deuteriums. Deuteroalkyl include, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl or C₂-C₁₅ heterocycloalkenyl), from two to ten carbon atoms (C₂-C₁₀ heterocycloalkyl or C₂-C₁₀ heterocycloalkenyl), from two to eight carbon atoms (C₂-C₈ heterocycloalkyl or C₂-C₈ heterocycloalkenyl), from two to seven carbon atoms (C₂-C₇ heterocycloalkyl or C₂-C₇ heterocycloalkenyl), from two to six carbon atoms (C₂-C₆ heterocycloalkyl or C₂-C₇ heterocycloalkenyl), from two to five carbon atoms (C₂-C₅ heterocycloalkyl or C₂-C₅ heterocycloalkenyl), or two to four carbon atoms (C₂-C₄ heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 12-membered heterocycloalkyl. In some embodiments, the heterocycloalkyl is a 3- to 10-membered heterocycloalkyl. In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 12-membered heterocycloalkenyl. In some embodiments, the heterocycloalkyl is a 3- to 10-membered heterocycloalkenyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to four heteroatoms selected from the group consisting of nitrogen, oxygen, and sulfur. In some embodiments, the heteroaryl comprises one to three heteroatoms selected from the group consisting of nitrogen, oxygen, and sulfur. In some embodiments, the heteroaryl comprises one to four heteroatoms selected from the group consisting of nitrogen and oxygen. In some embodiments, the heteroaryl comprises one to three heteroatoms selected from the group consisting of nitrogen and oxygen. In some embodiments, the heteroaryl comprises one to four nitrogens. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

The term “one or more” when referring to an optional substituent means that the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition.

“Synergy” or “synergize” refers to an effect of a combination that is greater than additive of the effects of each component alone at the same doses.

As used herein, the term “PTPN2-mediated” disorder or disease or alternatively “disease or disorder associated with PTPN2” means any disease or other deleterious condition in which PTPN2 or a mutant thereof is known to play a role. Accordingly, in some embodiments, the methods relate to treating or lessening the severity of one or more diseases in which PTPN2, or a mutant thereof, is known to play a role.

As used herein, the term “PTPN1-mediated” disorder or disease or alternatively “disease or disorder associated with PTPN1” means any disease or other deleterious condition in which PTPN1 or a mutant thereof is known to play a role. Accordingly, in some embodiments, the methods relate to treating or lessening the severity of one or more diseases in which PTPN1, or a mutant thereof, is known to play a role.

### Compounds

Described herein are compounds of Formula (I) and (II), or a pharmaceutically acceptable salt, solvate, or stereoisomer thereof, that are dual PTPN1/PTPN2 inhibitors.

Disclosed herein is a compound of Formula (I), or a pharmaceutically acceptable salt, solvate, or stereoisomer thereof:

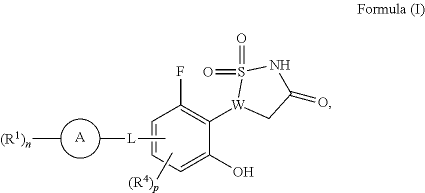

wherein:
Ring A is a heterocycloalkyl or heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R^{1a};
or two R¹ on the same atom are taken together to form an oxo;
or two R¹ on the same carbon are taken together to form a cycloalkyl or heterocycloalkyl; each optionally substituted with one or more R;
or two R¹ on the different atoms are taken together to form a cycloalkyl, heterocycloalkyl, aryl, or heteroaryl; each optionally substituted with one or more R;
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{a}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R;
or two R^{1a} on the same atom are taken together to form an oxo;
n is 0-11;
L is —O—, —S—, —S(═O)—, —S(═O)₂—, —NR²—, [C(R³)₂]ₘ—, —NR²S(═O)₂—, —S(═O)₂NR²—, —NR²C(═O)—, —C(═O)NR²—, —O[C(R³)₂]ₘ—, —NR²[C(R³)₂]ₘ—, —[C(R³)₂]ₘO—, or —[C(R³)₂]ₘNR²—;
R² is hydrogen, —C(═O)R^{a}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
each R³ is independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R;
or two R³ are taken together to form a cycloalkyl or heterocycloalkyl; each optionally substituted with one or more R;
m is 1-4;
each R⁴ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
p is 0-2;
W is CR^{W} or N;
R^{W} is hydrogen, deuterium, halogen, —CN, —NO₂, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{a}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently and optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently and optionally substituted with one or more R;
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently and optionally substituted with one or more R;
or R^{c} and R^{d} are taken together with the atom to which they are attached to form a heterocycloalkyl optionally substituted with one or more R; and
each R is independently halogen, —CN, —OH, —OC₁-C₃alkyl, —OC₁-C₃haloalkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃deuteroalkyl, C₁-C₃haloalkyl, C₁-C₃hydroxyalkyl, C₁-C₃aminoalkyl, C₁-C₃heteroalkyl, or C₃-C₆cycloalkyl;
or two R on the same atom form an oxo.

Disclosed herein is a compound of Formula (I), or a pharmaceutically acceptable salt, solvate, or stereoisomer thereof:

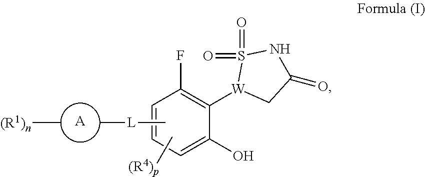

wherein:
Ring A is a heterocycloalkyl or heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{e}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R^{1a};
or two R¹ on the same atom are taken together to form an oxo;
or two R¹ on the same carbon are taken together to form a cycloalkyl or heterocycloalkyl; each optionally substituted with one or more R;
or two R¹ on the different atoms are taken together to form a cycloalkyl, heterocycloalkyl, aryl, or heteroaryl; each optionally substituted with one or more R;
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{a}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R;
or two R^{1a} on the same atom are taken together to form an oxo;
n is 0-11;
L is —O—, —S—, —S(═O)—, —S(═O)₂—, —NR²—, [C(R³)₂]ₘ—, —O[C(R³)₂]ₘ—, —NR²[C(R³)₂]ₘ, —[C(R³)₂]ₘO—, or —[C(R³)₂]ₘNR²—;
R² is hydrogen, —C(═O)R^{a}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
each R³ is independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R;
or two R³ are taken together to form a cycloalkyl or heterocycloalkyl; each optionally substituted with one or more R;
m is 1-4;
each R⁴ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
p is 0-2;
W is CR^{W} or N;
R^{W} is hydrogen, deuterium, halogen, —CN, —NO₂, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{a}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently and optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently and optionally substituted with one or more R;
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently and optionally substituted with one or more R;
or R^{c} and R^{d} are taken together with the atom to which they are attached to form a heterocycloalkyl optionally substituted with one or more R; and
each R is independently halogen, —CN, —OH, —OC₁-C₃alkyl, —OC₁-C₃haloalkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃deuteroalkyl, C₁-C₃haloalkyl, C₁-C₃hydroxyalkyl, C₁-C₃aminoalkyl, C₁-C₃heteroalkyl, or C₃-C₆cycloalkyl;
or two R on the same atom form an oxo.

In some embodiments of a compound of Formula (I), the compound is of Formula (Ia):

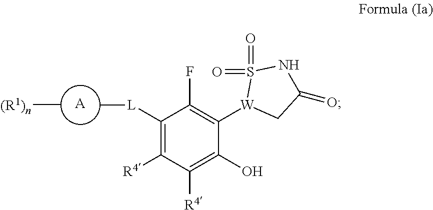

wherein R^{4′} is hydrogen or R⁴.

In some embodiments of a compound of Formula (I), the compound is of Formula (Ib):

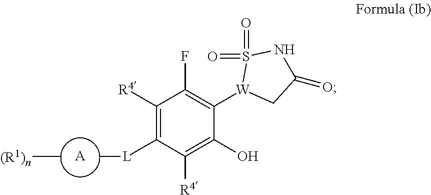

wherein R^{4′} is hydrogen or R⁴.

In some embodiments of a compound of Formula (I), the compound is of Formula (Ic):

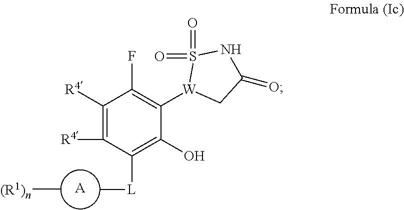

wherein R^{4′} is hydrogen or R⁴.

In some embodiments of a compound of Formula (I) or (Ia)-(Ic), Ring A is heterocycloalkyl. In some embodiments of a compound of Formula (I) or (Ia)-(Ic), Ring A is N-linked heterocycloalkyl. In some embodiments of a compound of Formula (I) or (Ia)-(Ic), Ring A is C-linked heterocycloalkyl. In some embodiments of a compound of Formula (I) or (Ia)-(Ic), Ring A is 4- to 8-membered heterocycloalkyl. In some embodiments of a compound of Formula (I) or (Ia)-(Ic), Ring A is 5- to 8-membered heterocycloalkyl. In some embodiments of a compound of Formula (I) or (Ia)-(Ic), Ring A is 5- to 6-membered heterocycloalkyl. In some embodiments of a compound of Formula (I) or (Ia)-(Ic), Ring A is 5-membered heterocycloalkyl. In some embodiments of a compound of Formula (I) or (Ia)-(Ic), Ring A is 6-membered heterocycloalkyl. In some embodiments of a compound of Formula (I) or (Ia)-(Ic), Ring A is heterocycloalkyl comprising 1 to 4 heteroatoms selected from the group consisting of O, S, and N. In some embodiments of a compound of Formula (I) or (Ia)-(Ic), Ring A is heterocycloalkyl comprising 1 to 3 heteroatoms selected from the group consisting of O, S, and N. In some embodiments of a compound of Formula (I) or (Ia)-(Ic), Ring A is heterocycloalkyl comprising 1 to 3 heteroatoms selected from the group consisting of O and N. In some embodiments of a compound of Formula (I) or (Ia)-(Ic), Ring A is heterocycloalkyl comprising 1 to 2 heteroatoms selected from the group consisting of O and N. In some embodiments of a compound of Formula (I) or (Ia)-(Ic), Ring A is heterocycloalkyl comprising 1 to 2 heteroatoms that are N. In some embodiments of a compound of Formula (I) or (Ia)-(Ic), Ring A is heterocycloalkyl comprising 1 heteroatom that is N.

In some embodiments of a compound of Formula (I) or (Ia)-(Ic), Ring A is monocyclic heterocycloalkyl. In some embodiments of a compound of Formula (I) or (Ia)-(Ic), Ring A is azetidinyl, pyrrolidinyl, piperidinyl, piperazinyl, morpholinyl, or azepanyl. In some embodiments of a compound of Formula (I) or (Ia)-(Ic), Ring A is pyrrolidinyl or piperidinyl. In some embodiments of a compound of Formula (I) or (Ia)-(Ic), Ring A is pyrrolidinyl. In some embodiments of a compound of Formula (I) or (Ia)-(Ic), Ring A is piperidinyl.

In some embodiments of a compound of Formula (I) or (Ia)-(Ic), Ring A is bicyclic heterocycloalkyl.

In some embodiments of a compound of Formula (I) or (Ia)-(Ic), Ring A is 6-azaspiro[3.4]octanyl, 7-azaspiro[3.5]nonanyl, 6-azaspiro[2.5]octanyl, 2-azaspiro[4.4]nonanyl, 8-oxa-2-azaspiro[4.5]decanyl, 2-azaspiro[3.4]octanyl, 2-oxa-7-azaspiro[4.4]nonanyl, 2-azaspiro[4.5]decanyl, or 2-azaspiro[3.3]heptanyl.

In some embodiments of a compound of Formula (I) or (Ia)-(Ic), Ring A is heteroaryl. In some embodiments of a compound of Formula (I) or (Ia)-(Ic), Ring A is 5- or 6-membered heteroaryl. In some embodiments of a compound of Formula (I) or (Ia)-(Ic), Ring A is 5-membered heteroaryl. In some embodiments of a compound of Formula (I) or (Ia)-(Ic), Ring A is 6-membered heteroaryl.

In some embodiments of a compound of Formula (I) or (Ia)-(Ic), Ring A is heteroaryl comprising 1 to 4 heteroatoms selected from the group consisting of O, S, and N. In some embodiments of a compound of Formula (I) or (Ia)-(Ic), Ring A is heteroaryl comprising 1 to 3 heteroatoms selected from the group consisting of O, S, and N. In some embodiments of a compound of Formula (I) or (Ia)-(Ic), Ring A is heteroaryl comprising 1 to 3 heteroatoms selected from the group consisting of O and N. In some embodiments of a compound of Formula (I) or (Ia)-(Ic), Ring A is heteroaryl comprising 1 to 2 heteroatoms selected from the group consisting of O and N. In some embodiments of a compound of Formula (I) or (Ia)-(Ic), Ring A is heteroaryl comprising 1 to 2 heteroatoms that are N. In some embodiments of a compound of Formula (I) or (Ia)-(Ic), Ring A is heteroaryl comprising 1 heteroatom that is N.

In some embodiments of a compound of Formula (I) or (Ia)-(Ic), Ring A is

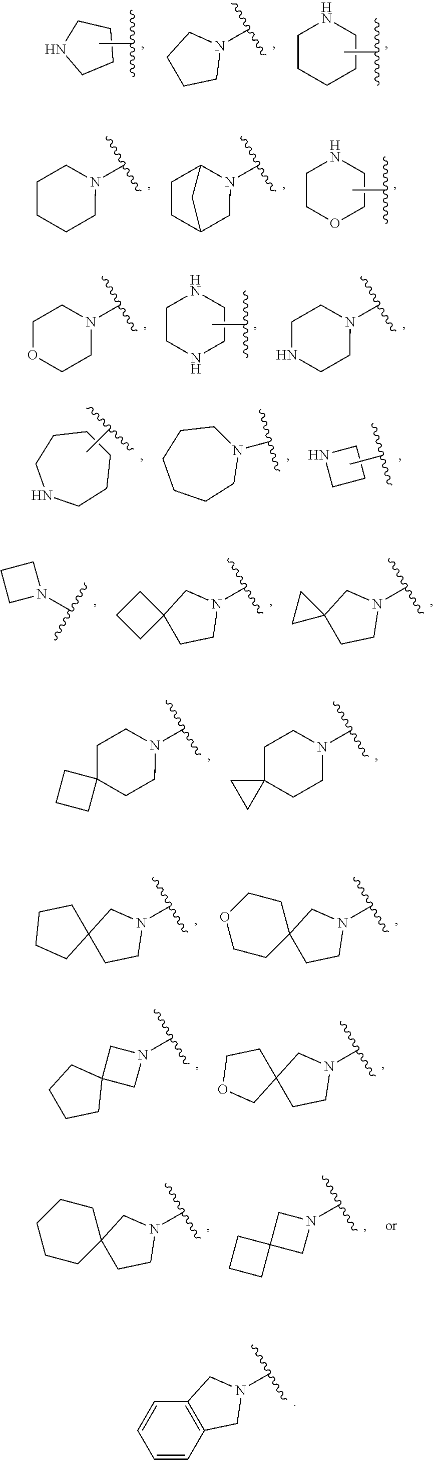

In some embodiments of a compound of Formula (I) or (Ia)-(Ic), Ring A is

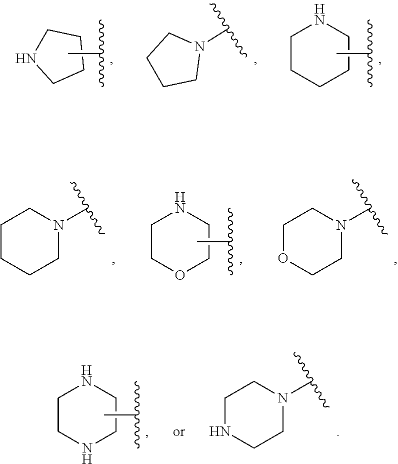

In some embodiments of a compound of Formula (I) or (Ia)-(Ic), Ring A is

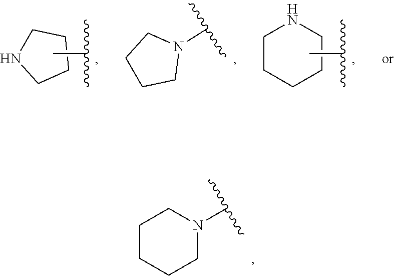

In some embodiments of a compound of Formula (I) or (Ia)-(Ic), Ring A is

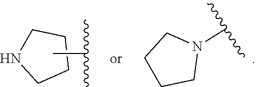

In some embodiments of a compound of Formula (I) or (Ia)-(Ic), Ring A is

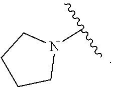 [SMILES: *N1CCCC1]

In some embodiments of a compound of Formula (I) or (Ia)-(Ic), Ring A is

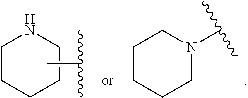

In some embodiments of a compound of Formula (I) or (Ia)-(Ic), Ring A is

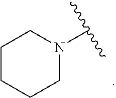 [SMILES: *N1CCCCC1]

In some embodiments of a compound of Formula (I) or (Ia)-(Ic), Ring A is,

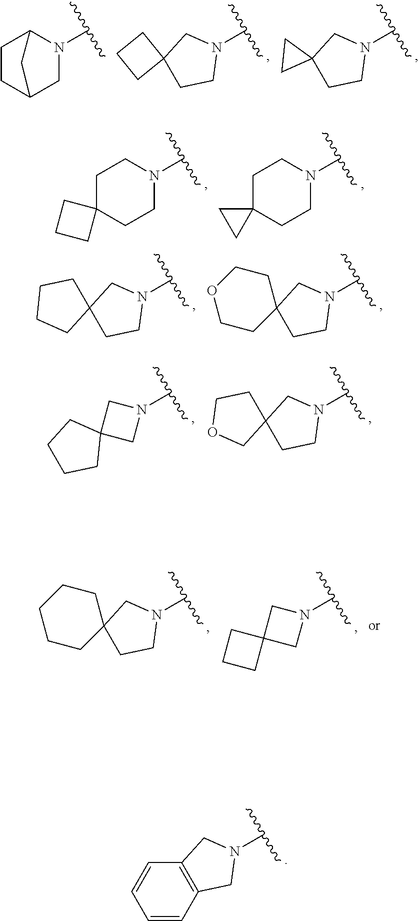

In some embodiments of a compound of Formula (I) or (Ia)-(Ic), Ring A is

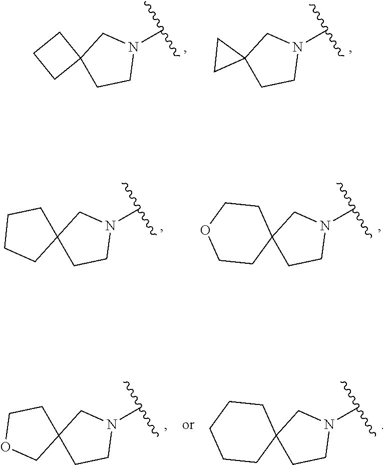 [SMILES: *N1CCC2(CCC2)C1 ; *N1CCC2(CC2)C1 ; *N1CCC2(CCCC2)C1 ; *N1CCC2(CCOCC2)C1 ; *N1CCC2(CCOC2)C1 ; *N1CCC2(CCCCC2)C1]

In some embodiments of a compound of Formula (I) or (Ia)-(Ic), Ring A is

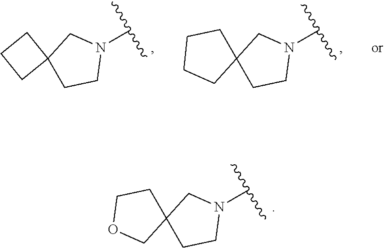 [SMILES: *N1CCC2(CCC2)C1 ; *N1CCC2(CCCC2)C1 ; *N1CCC2(CCOC2)C1]

In some embodiments of a compound of Formula (I) or (Ia)-(Ic), Ring A is

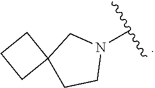 [SMILES: *N1CCC2(CCC2)C1]

In some embodiments of a compound of Formula (I) or (Ia)-(Ic), Ring A is

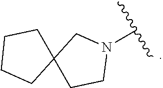 [SMILES: *N1CCC2(CCCC2)C1]

In some embodiments of a compound of Formula (I) or (Ia)-(Ic), Ring A is

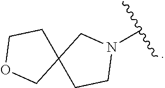 [SMILES: *N1CCC2(CCOC2)C1]

In some embodiments of a compound of Formula (I) or (Ia)-(Ic), Ring A is

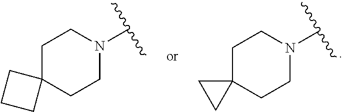 [SMILES: *N1CCC2(CCC2)CC1 ; *N1CCC2(CC1)CC2]

In some embodiments of a compound of Formula (I) or (Ia)-(Ic), L is —O—, —S—, —S(═O)—, —S(═O)₂—, —NR²—, [(R³)₂]ₘ—, —O[C(R³)₂]ₘ, —NR²[C(R³)₂]ₘ—, —[C(R³)₂]ₘO—, or —[C(R³)₂]ₘNR²—.

In some embodiments of a compound of Formula (I) or (Ia)-(Ic), L is —[C(R³)₂]—.

In some embodiments of a compound of Formula (I) or (Ia)-(Ic), each R³ is independently hydrogen, deuterium, halogen, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; or two R³ are taken together to form a cycloalkyl. In some embodiments of a compound of Formula (I) or (Ia)-(Ic), each R³ is independently hydrogen, deuterium, halogen, —OH, C₁-C₆alkyl, or C₁-C₆haloalkyl; or two R³ are taken together to form a cycloalkyl. In some embodiments of a compound of Formula (I) or (Ia)-(Ic), each R³ is independently hydrogen, halogen, —OH, or C₁-C₆alkyl; or two R³ are taken together to form a cycloalkyl. In some embodiments of a compound of Formula (I) or (Ia)-(Ic), two R³ are taken together to form a cycloalkyl or heterocycloalkyl; each optionally substituted with one or more R. In some embodiments of a compound of Formula (I) or (Ia)-(Ic), two R³ are taken together to form a cycloalkyl optionally substituted with one or more R.

In some embodiments of a compound of Formula (I) or (Ia)-(Ic), each R³ is independently hydrogen, deuterium, halogen, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl. In some embodiments of a compound of Formula (I) or (Ia)-(Ic), each R³ is independently hydrogen, deuterium, halogen, —OH, C₁-C₆alkyl, or C₁-C₆haloalkyl. In some embodiments of a compound of Formula (I) or (Ia)-(Ic), each R³ is independently hydrogen, C₁-C₆alkyl, or C₁-C₆haloalkyl. In some embodiments of a compound of Formula (I) or (Ia)-(Ic), each R³ is independently hydrogen or C₁-C₆alkyl. In some embodiments of a compound of Formula (I) or (Ia)-(Ic), each R³ is hydrogen.

In some embodiments of a compound of Formula (I) or (Ia)-(Ic), m is 1-3. In some embodiments of a compound of Formula (I) or (Ia)-(Ic), m is 1 or 2. In some embodiments of a compound of Formula (I) or (Ia)-(Ic), m is 1. In some embodiments of a compound of Formula (I) or (Ia)-(Ic), m is 2.

In some embodiments of a compound of Formula (I) or (Ia)-(Ic), L is —CH₂—, —CH₂CH₂—, or —CH₂CH₂CH₂—. In some embodiments of a compound of Formula (I) or (Ia)-(Ic), L is —CH₂—. In some embodiments of a compound of Formula (I) or (Ia)-(Ic), L is —CH₂CH₂—. In some embodiments of a compound of Formula (I) or (Ia)-(Ic), L is —CH₂CH₂CH₂—.

In some embodiments of a compound of Formula (I) or (Ia)-(Ic), L is —O—. In some embodiments of a compound of Formula (I) or (Ia)-(Ic), L is —S—, —S(═O)—, or —S(═O)₂—. In some embodiments of a compound of Formula (I) or (Ia)-(Ic), L is —NR²—.

In some embodiments of a compound of Formula (I) or (Ia)-(Ic), L is —NR²C(═O)— or —C(═O)NR²—.

In some embodiments of a compound of Formula (I) or (Ia)-(Ic), L is —NR²S(═O)₂— or —S(═O)₂NR²—. In some embodiments of a compound of Formula (I) or (Ia)-(Ic), L is —O[C(R³)₂]ₘ, —NR²[C(R³)₂]ₘ, —[C(R³)₂]ₘO—, or [C(R³)₂]ₘNR²—. In some embodiments of a compound of Formula (I) or (Ia)-(Ic), L is —O[C(R³)₂]ₘ— or —NR²[C(R³)₂]ₘ—. In some embodiments of a compound of Formula (I) or (Ia)-(Ic), L is —[C(R³)₂]O— or —[C(R³)₂]ₘNR²—.

In some embodiments of a compound of Formula (I) or (Ia)-(Ic), R² is hydrogen, C₁-C₆alkyl, C₁-C₆haloalkyl, C₁-C₆deuteroalkyl, cycloalkyl, or heterocycloalkyl. In some embodiments of a compound of Formula (I) or (Ia)-(Ic), R² is hydrogen, C₁-C₆alkyl, or C₁-C₆haloalkyl. In some embodiments of a compound of Formula (I) or (Ia)-(Ic), R² is hydrogen or C₁-C₆alkyl.

In some embodiments of a compound of Formula (I) or (Ia)-(Ic), each R⁴ is independently deuterium, halogen, C₁-C₆alkyl, or C₁-C₆haloalkyl. In some embodiments of a compound of Formula (I) or (Ia)-(Ic), each R⁴ is independently deuterium or halogen. In some embodiments of a compound of Formula (I) or (Ia)-(Ic), each R⁴ is independently halogen. In some embodiments of a compound of Formula (I) or (Ia)-(Ic), each R⁴ is independently —OR^{a} or C₁-C₆alkyl.

In some embodiments of a compound of Formula (I) or (Ia)-(Ic), p is 0 or 1. In some embodiments of a compound of Formula (I) or (Ia)-(Ic), p is 0. In some embodiments of a compound of Formula (I) or (Ia)-(Ic), p is 1.

In some embodiments of a compound of Formula (I) or (Ia)-(Ic), W is N. In some embodiments of a compound of Formula (I) or (Ia)-(Ic), W is CR^{W}.

In some embodiments of a compound of Formula (I) or (Ia)-(Ic), R^{W} is hydrogen, deuterium, halogen, or C₁-C₆alkyl. In some embodiments of a compound of Formula (I) or (Ia)-(Ic), R^{W} is hydrogen or C₁-C₆alkyl. In some embodiments of a compound of Formula (I) or (Ia)-(Ic), R^{W} is hydrogen. In some embodiments of a compound of Formula (I) or (Ia)-(Ic), R^{W} is C₁-C₆alkyl.

In some embodiments of a compound of Formula (I) or (Ia)-(Ic), each R¹ is independently deuterium, halogen, —CN, —OH, —OR^{a}, —NR^{c}R^{d}, —C(═O)R^{a}, —C(═O)OR^{a}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R^{1a}. In some embodiments of a compound of Formula (I) or (Ia)-(Ic), each R¹ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R^{1a}. In some embodiments of a compound of Formula (I) or (Ia)-(Ic), each R¹ is independently deuterium, halogen, —OH, —OR^{a}, C₁-C₆alkyl, C₁-C₆haloalkyl, C₁-C₆hydroxyalkyl, C₁-C₆heteroalkyl, or cycloalkyl; wherein each alkyl and cycloalkyl is independently and optionally substituted with one or more R^{1a}. In some embodiments of a compound of Formula (I) or (Ia)-(Ic), each R¹ is independently halogen, —OH, —OR^{a}, C₁-C₆alkyl, C₁-C₆haloalkyl, C₁-C₆hydroxyalkyl, C₁-C₆heteroalkyl, or cycloalkyl. In some embodiments of a compound of Formula (I) or (Ia)-(Ic), each R¹ is independently halogen, C₁-C₆alkyl, or C₁-C₆haloalkyl. In some embodiments of a compound of Formula (I) or (Ia)-(Ic), each R¹ is independently halogen or C₁-C₆alkyl. In some embodiments of a compound of Formula (I) or (Ia)-(Ic), each R¹ is independently C₁-C₆alkyl. In some embodiments of a compound of Formula (I) or (Ia)-(Ic), each R¹ is independently C₁-C₆alkyl optionally substituted with one or more R¹.

In some embodiments of a compound of Formula (I) or (Ia)-(Ic), each R¹ is independently deuterium, halogen, —CN, —OH, —OR^{a}, —S(═O)₂R^{a}, —S(═O)₂NR^{c}R^{d}, —NR^{c}R^{d}, —C(═O)R^{a}, —C(═O)OR^{a}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R^{1a}.

In some embodiments of a compound of Formula (I) or (Ia)-(Ic), each R¹ is independently deuterium, halogen, —CN, —OH, —OR^{a}, —S(═O)₂R^{a}, —S(═O)₂NR^{c}R^{d}, —NR^{c}R^{d}, —C(═O)R^{a}, —C(═O)OR^{a}, —C(═O)NR^{c}R^{d}, C₁-C₆alkyl, C₁-C₆haloalkyl, C₁-C₆deuteroalkyl, cycloalkyl, or heterocycloalkyl; wherein each alkyl, cycloalkyl, and heterocycloalkyl is independently and optionally substituted with one or more R^{1a}. In some embodiments of a compound of Formula (I) or (Ia)-(Ic), each R¹ is independently deuterium, halogen, —CN, —OH, —OR^{a}, —S(═O)₂R^{a}, —S(═O)₂NR^{c}R^{d}, —NR^{c}R^{d}, —C(═O)R^{a}, —C(═O)OR^{a}, —C(═O)NR^{c}R^{d}, C₁-C₆alkyl, C₁-C₆haloalkyl, C₁-C₆deuteroalkyl. In some embodiments of a compound of Formula (I) or (Ia)-(Ic), each R^{a} is independently deuterium, halogen, —S(═O)₂R^{a}, —S(═O)₂NR^{c}R^{d}, —C(═O)R^{a}, —C(═O)OR^{a}, —C(═O)NR^{c}R^{d}, C₁-C₆alkyl, C₁-C₆haloalkyl, C₁-C₆deuteroalkyl. In some embodiments of a compound of Formula (I) or (Ia)-(Ic), each R¹ is independently C₁-C₆alkyl or C₁-C₆haloalkyl. In some embodiments of a compound of Formula (I) or (Ia)-(Ic), each R¹ is independently C₁-C₆alkyl. In some embodiments of a compound of Formula (I) or (Ia)-(Ic), two R¹ on the same atom are taken together to form an oxo. In some embodiments of a compound of Formula (I) or (Ia)-(Ic), two R¹ on the same carbon are taken together to form a cycloalkyl or heterocycloalkyl; each optionally substituted with one or more R. In some embodiments of a compound of Formula (I) or (Ia)-(Ic), two R¹ on the different atoms are taken together to form a cycloalkyl, heterocycloalkyl, aryl, or heteroaryl; each optionally substituted with one or more R.

In some embodiments of a compound of Formula (I) or (Ia)-(Ic), each R^{1a} is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R. In some embodiments of a compound of Formula (I) or (Ia)-(Ic), each R^{1a} is independently deuterium, halogen, —CN, —OH, —OR^{a}, —NR^{c}R^{d}, C₁-C₆alkyl, C₁-C₆haloalkyl, cycloalkyl, heterocycloalkyl, aryl, or heteroaryl; wherein each alkyl, cycloalkyl, heterocycloalkyl, aryl, and heteroaryl is independently and optionally substituted with one or more R. In some embodiments of a compound of Formula (I) or (Ia)-(Ic), each R^{1a} is independently deuterium, halogen, —CN, —OH, —OR^{a}, —NR^{c}R^{d}, C₁-C₆alkyl, C₁-C₆haloalkyl, cycloalkyl, heterocycloalkyl, aryl, or heteroaryl. In some embodiments of a compound of Formula (I) or (Ia)-(Ic), each R^{1a} is independently halogen, —NR^{c}R^{d}, C₁-C₆alkyl, C₁-C₆haloalkyl, cycloalkyl, heterocycloalkyl, aryl, or heteroaryl. In some embodiments of a compound of Formula (I) or (Ia)-(Ic), each R^{1a} is independently —NR^{c}R^{d}, cycloalkyl, heterocycloalkyl, aryl, or heteroaryl. In some embodiments of a compound of Formula (I) or (Ia)-(Ic), each R^{1a} is independently cycloalkyl, heterocycloalkyl, aryl, or heteroaryl.

In some embodiments of a compound of Formula (I) or (Ia)-(Ic), each R¹ is independently fluoro,

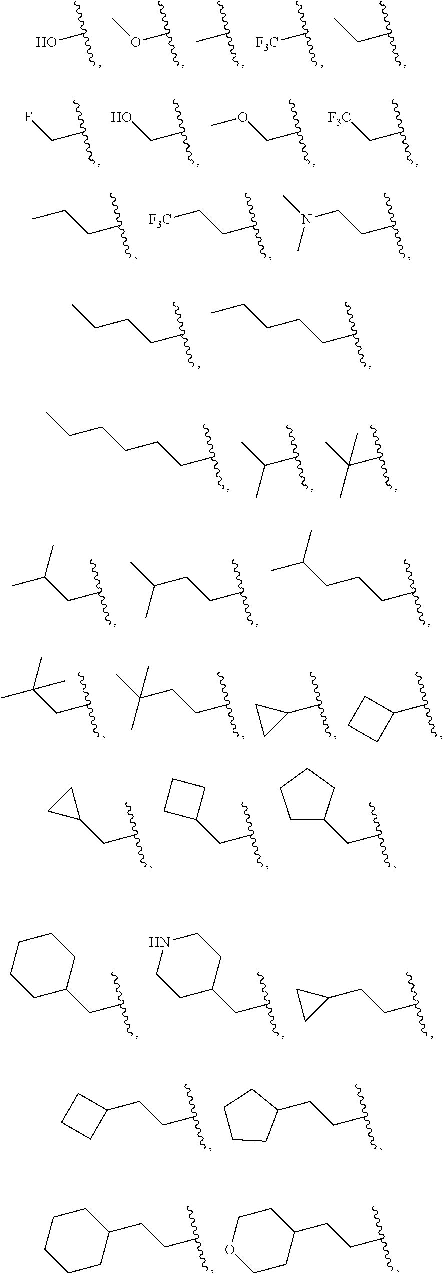

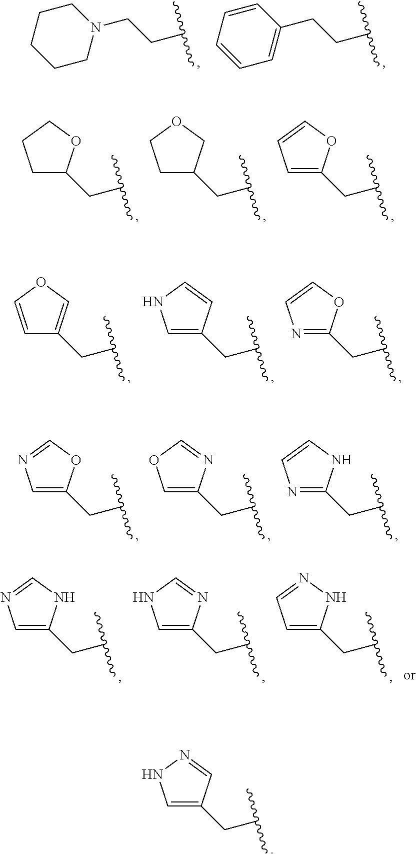

In some embodiments of a compound of Formula (I) or (Ia)-(Ic), n is 0-8. In some embodiments of a compound of Formula (I) or (Ia)-(Ic), n is 0-7. In some embodiments of a compound of Formula (I) or (Ia)-(Ic), n is 0-6. In some embodiments of a compound of Formula (I) or (Ia)-(Ic), n is 0-5. In some embodiments of a compound of Formula (I) or (Ia)-(Ic), n is 0-4. In some embodiments of a compound of Formula (I) or (Ia)-(Ic), n is 0-3. In some embodiments of a compound of Formula (I) or (Ia)-(Ic), n is 0-2. In some embodiments of a compound of Formula (I) or (Ia)-(Ic), n is 0 or 1. In some embodiments of a compound of Formula (I) or (Ia)-(Ic), n is 0. In some embodiments of a compound of Formula (I) or (Ia)-(Ic), n is 1-8. In some embodiments of a compound of Formula (I) or (Ia)-(Ic), n is 1-7. In some embodiments of a compound of Formula (I) or (Ia)-(Ic), n is 1-6. In some embodiments of a compound of Formula (I) or (Ia)-(Ic), n is 1-5. In some embodiments of a compound of Formula (I) or (Ia)-(Ic), n is 1-4. In some embodiments of a compound of Formula (I) or (Ia)-(Ic), n is 1-3. In some embodiments of a compound of Formula (I) or (Ia)-(Ic), n is 1 or 2. In some embodiments of a compound of Formula (I) or (Ia)-(Ic), n is 1. In some embodiments of a compound of Formula (I) or (Ia)-(Ic), n is 2. In some embodiments of a compound of Formula (I) or (Ia)-(Ic), n is 3. In some embodiments of a compound of Formula (I) or (Ia)-(Ic), n is 4. In some embodiments of a compound of Formula (I) or (Ia)-(Ic), n is 5. In some embodiments of a compound of Formula (I) or (Ia)-(Ic), n is 6.

In some embodiments of a compound of Formula (I) or (Ia)-(Ic),

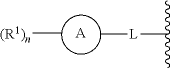

is

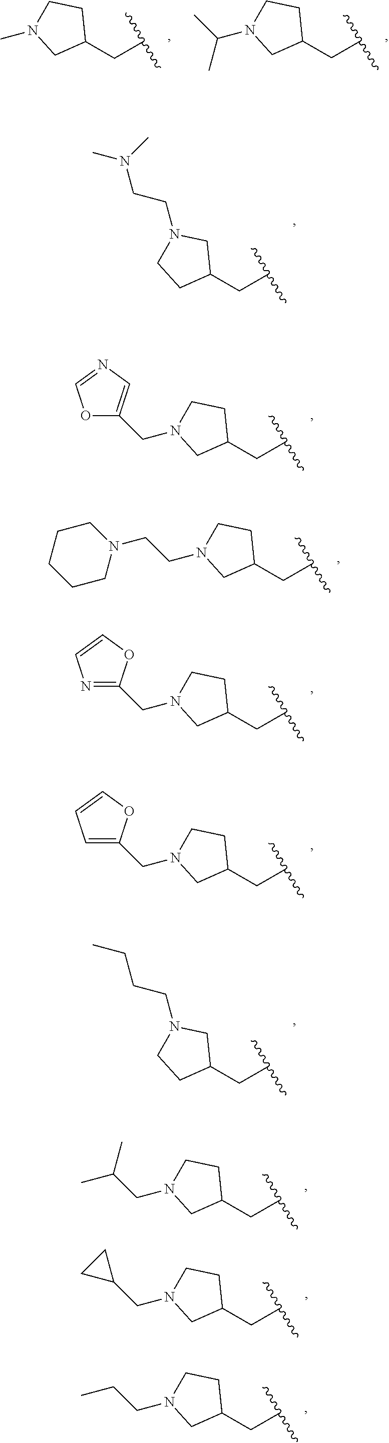

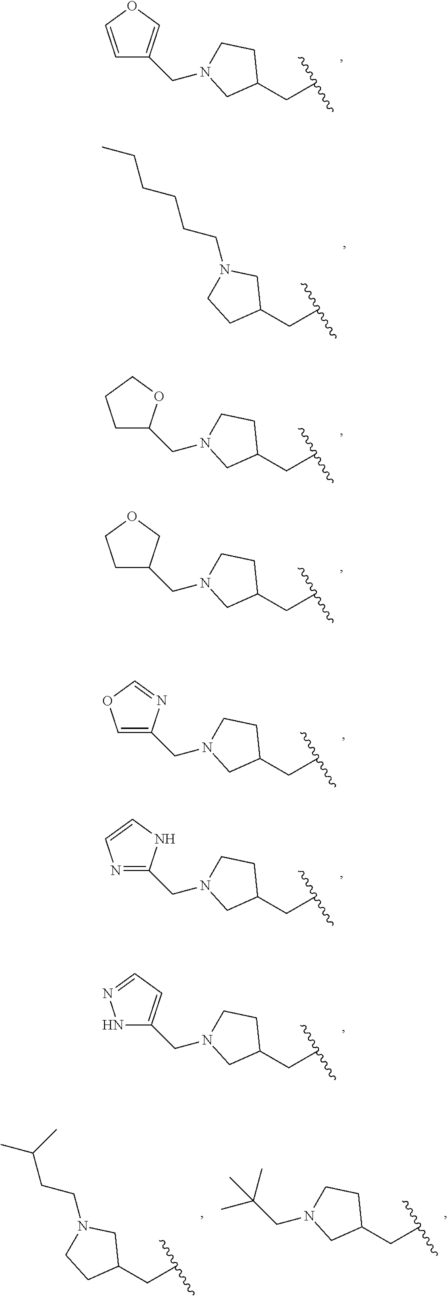

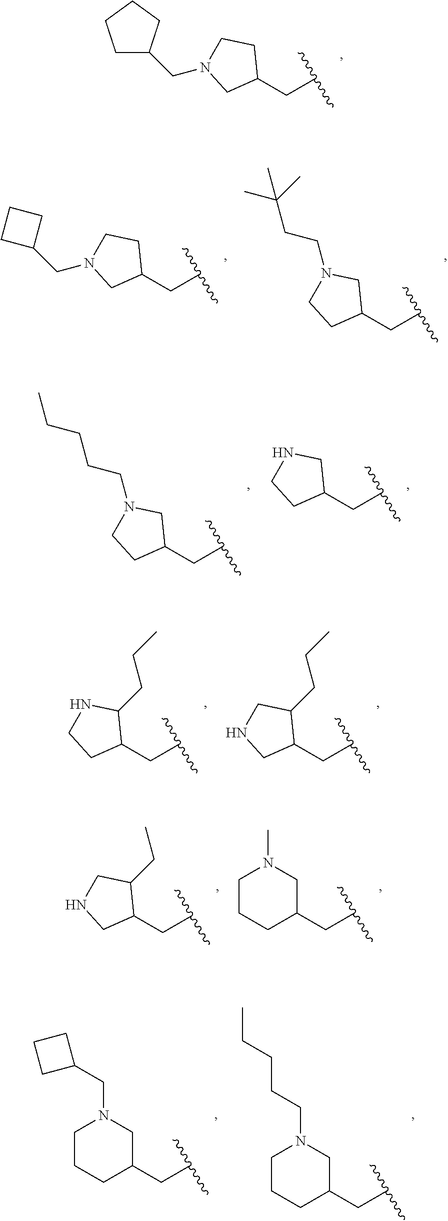

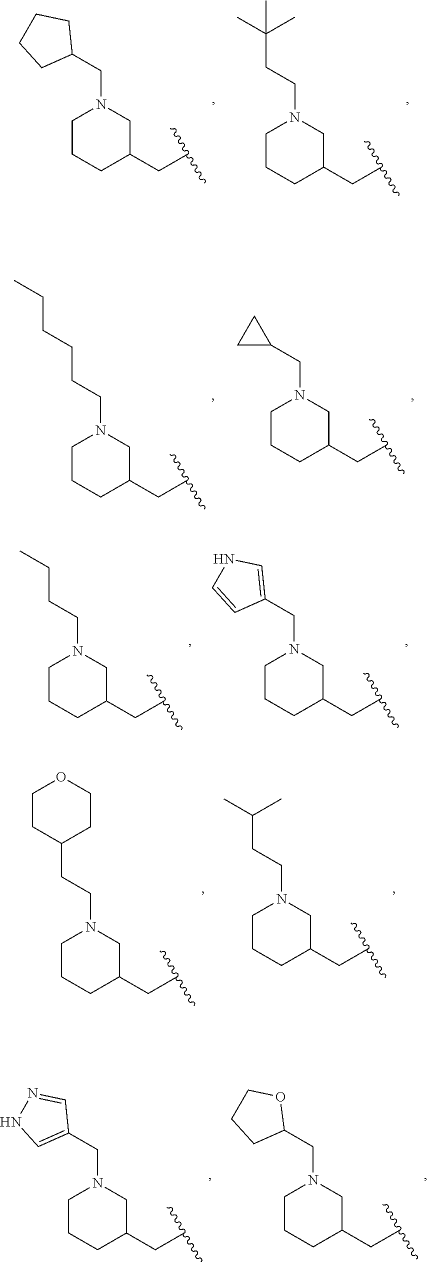

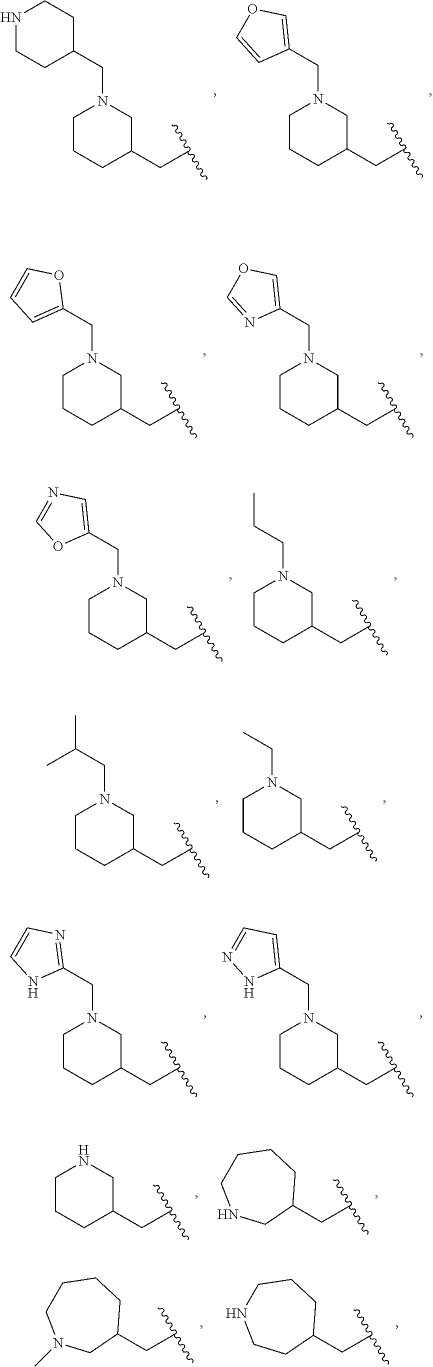

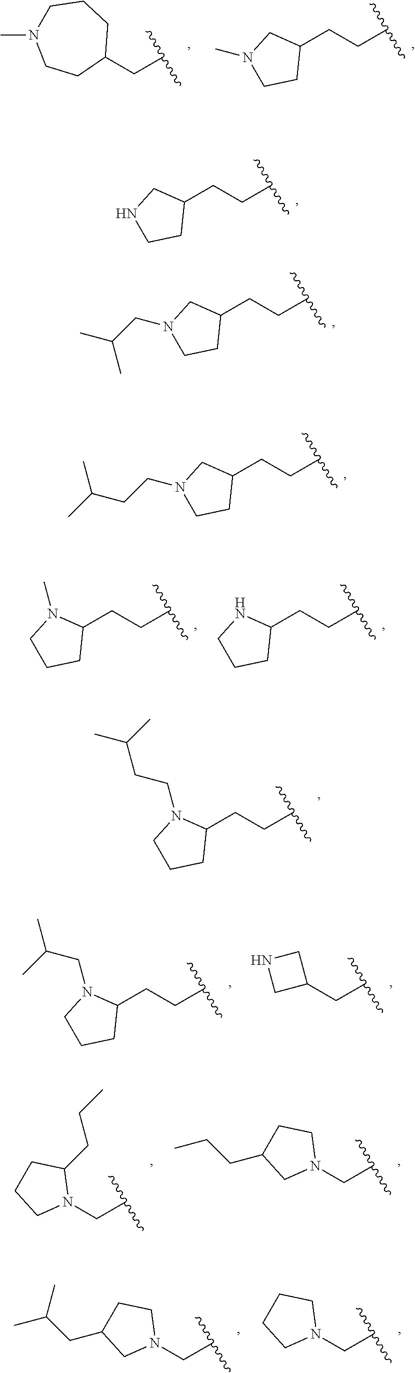

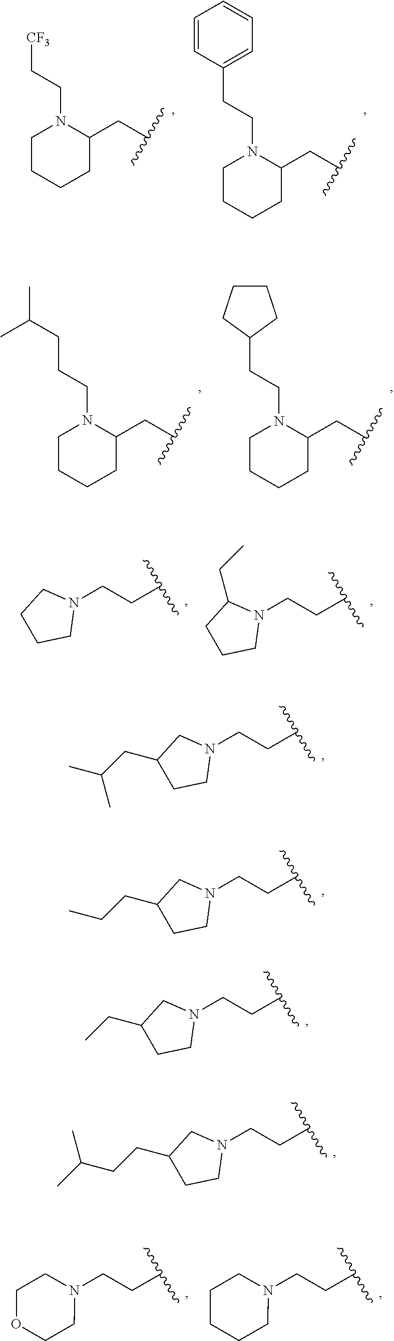

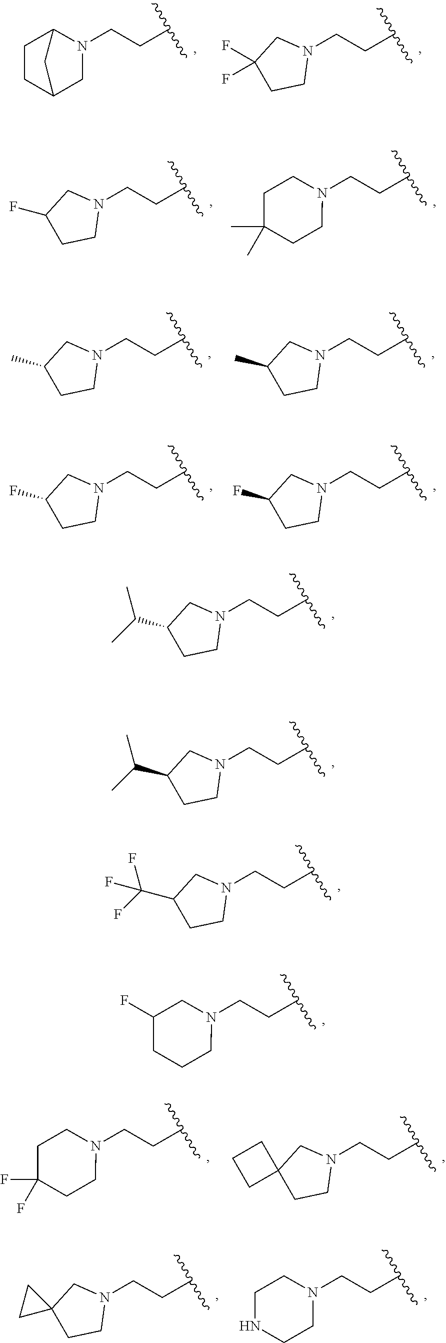

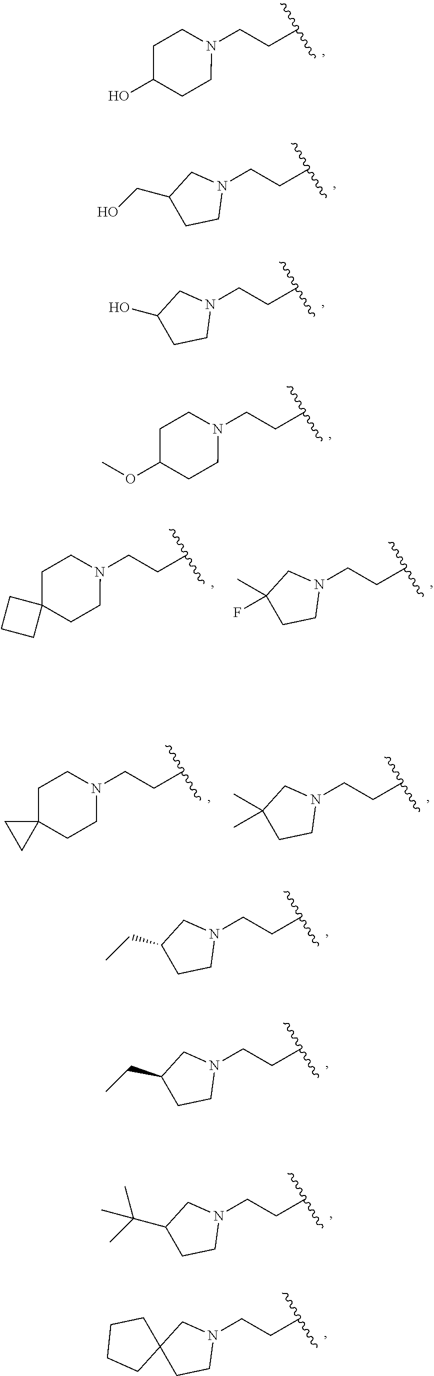

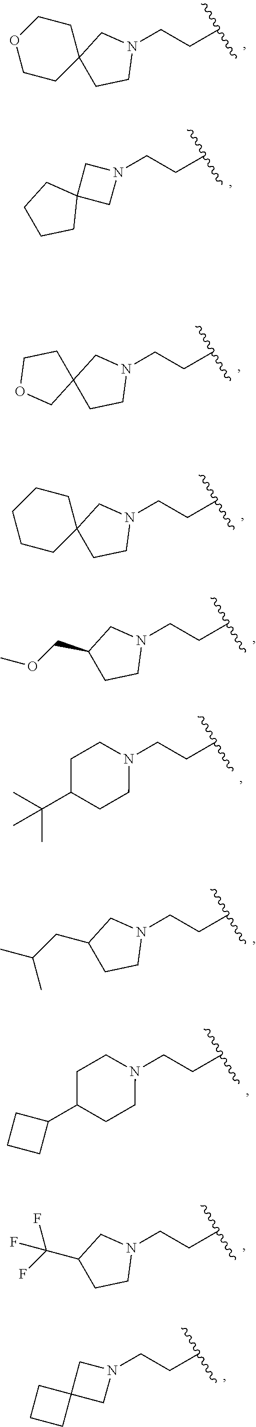

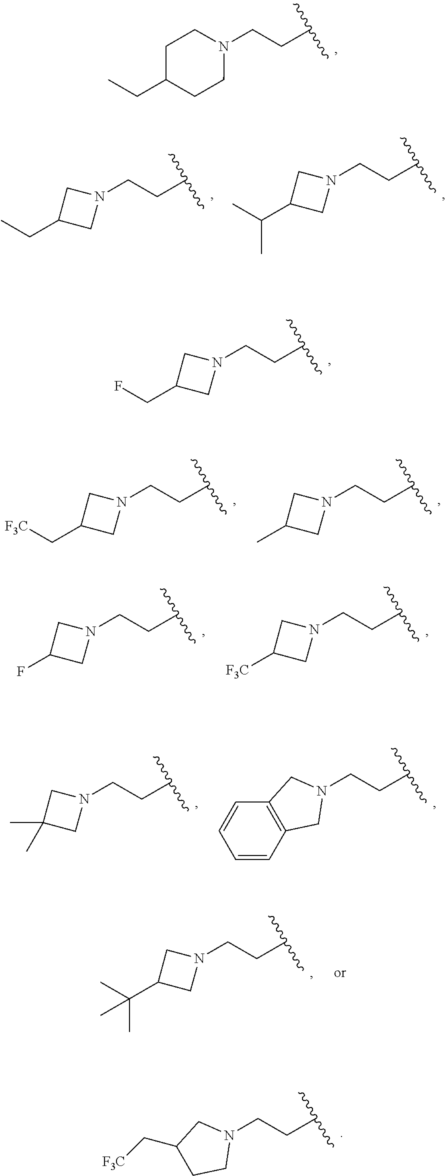

In some embodiments of a compound of Formula (I) or (Ia)-(Ic),

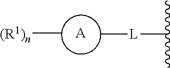

is

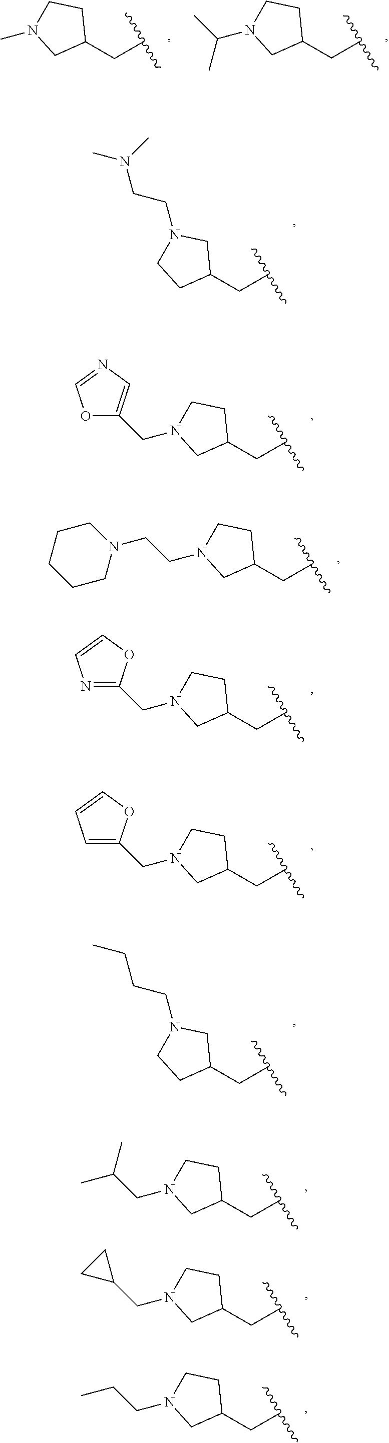

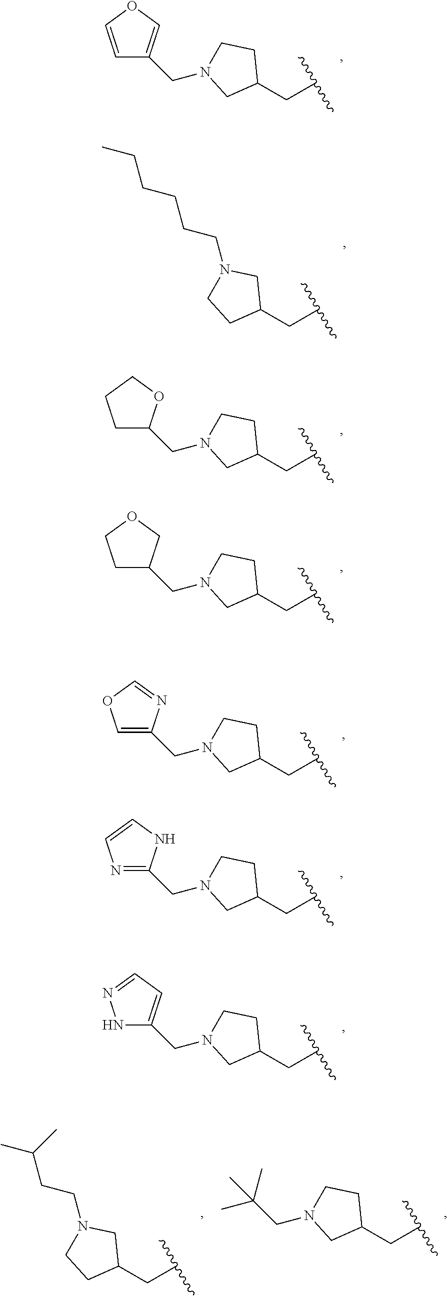

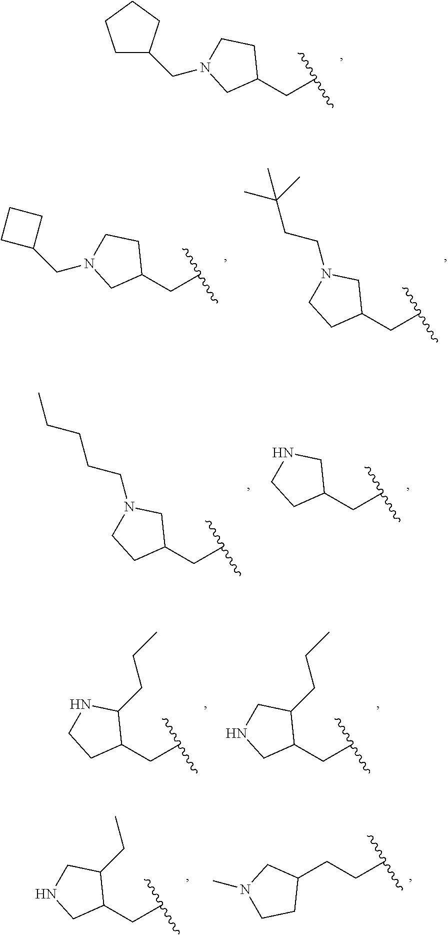

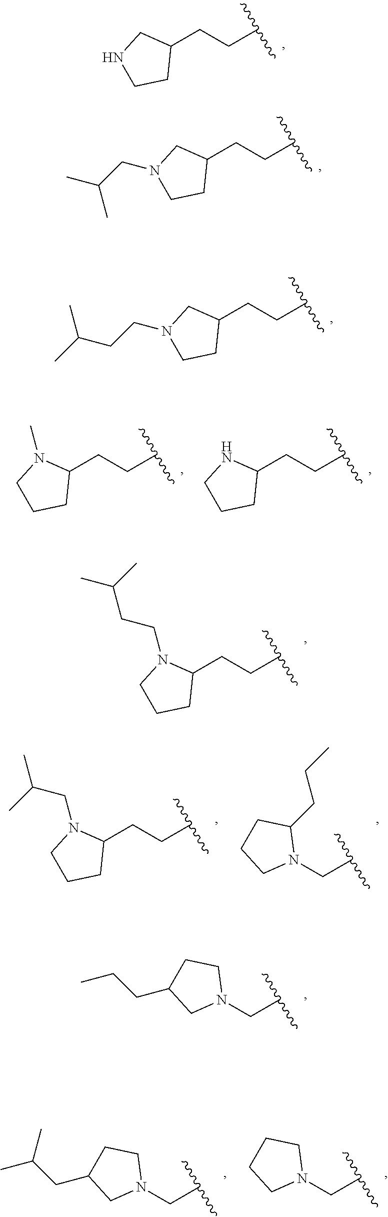

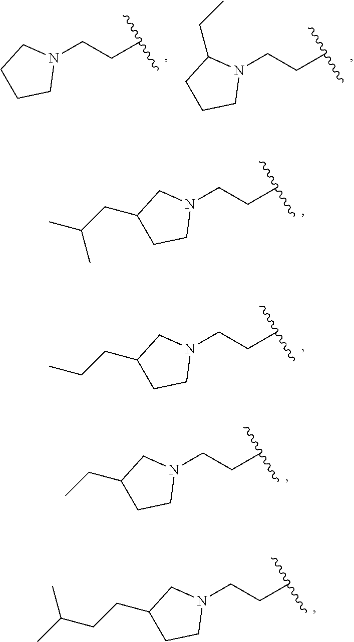

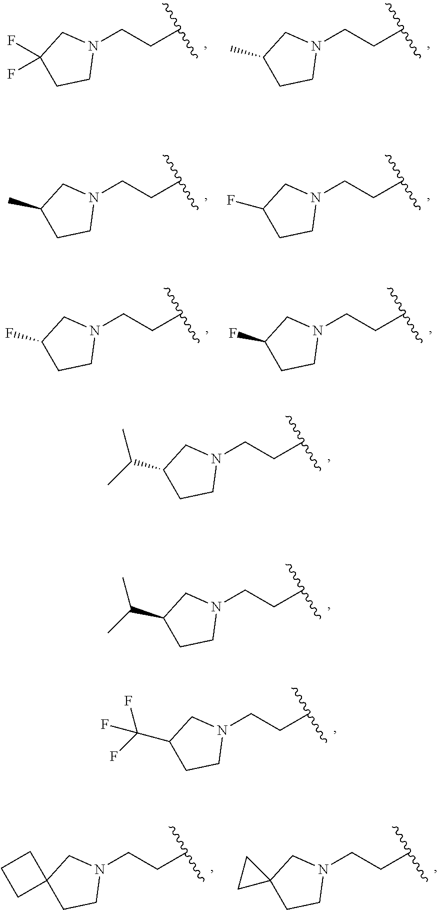

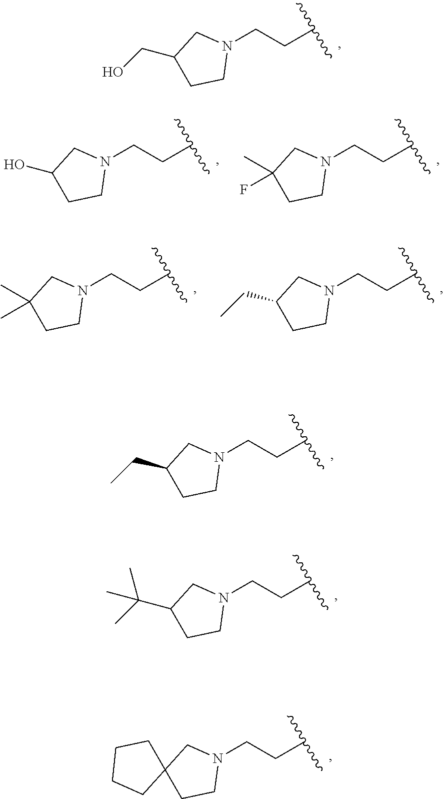

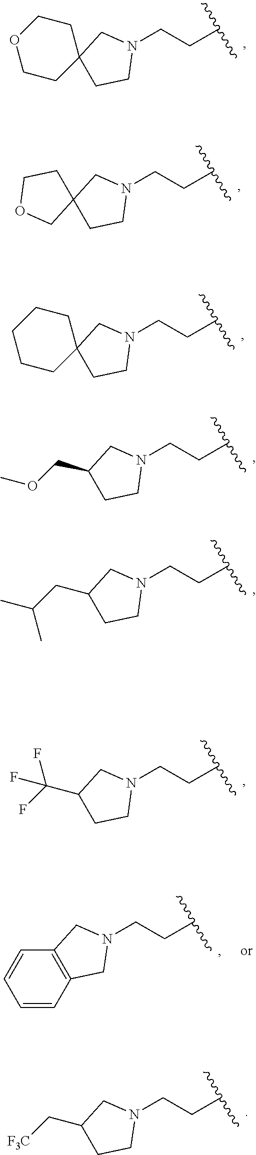

In some embodiments of a compound of Formula (I) or (Ia)-(Ic),

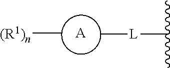

is

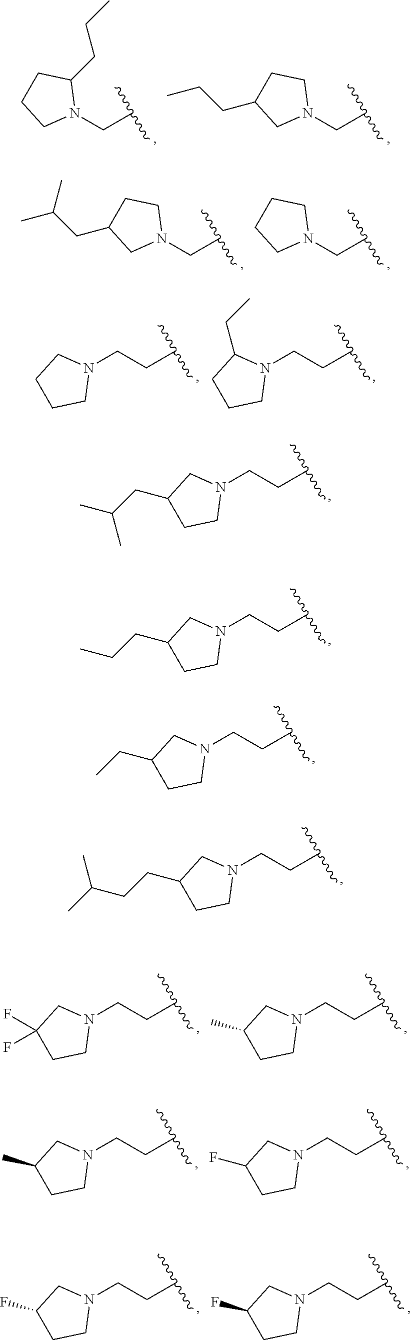

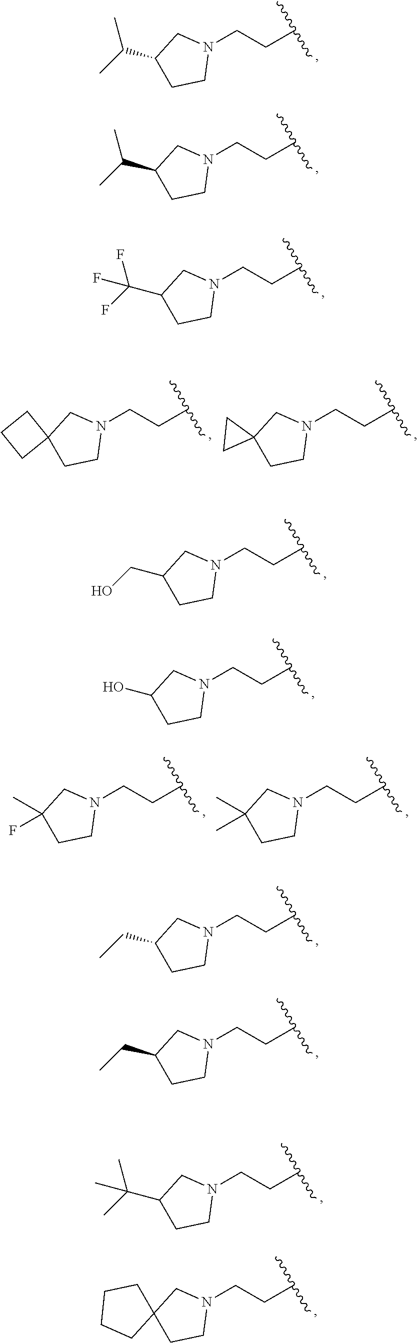

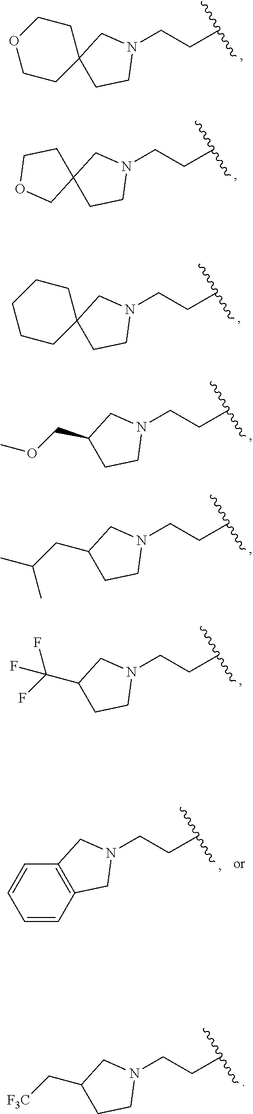

In some embodiments of a compound of Formula (I) or (Ia)-(Ic),

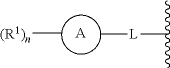

is

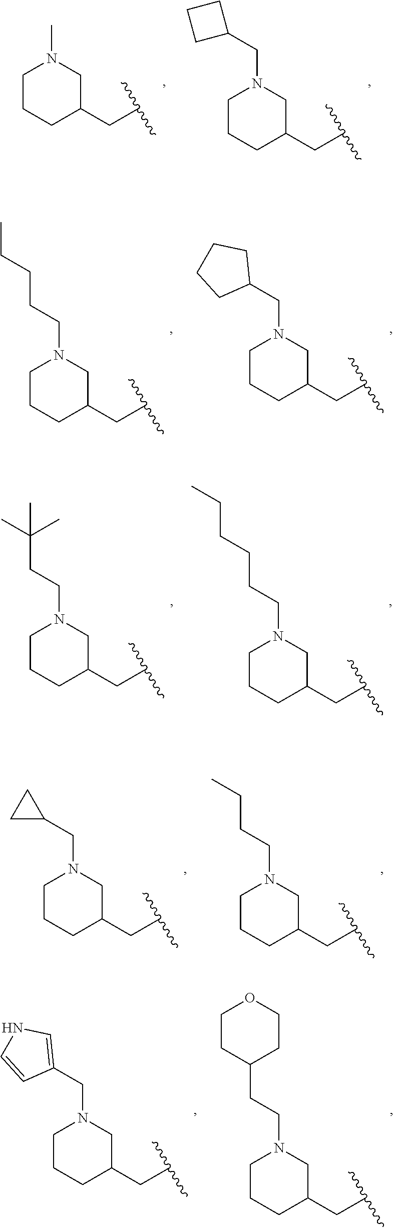

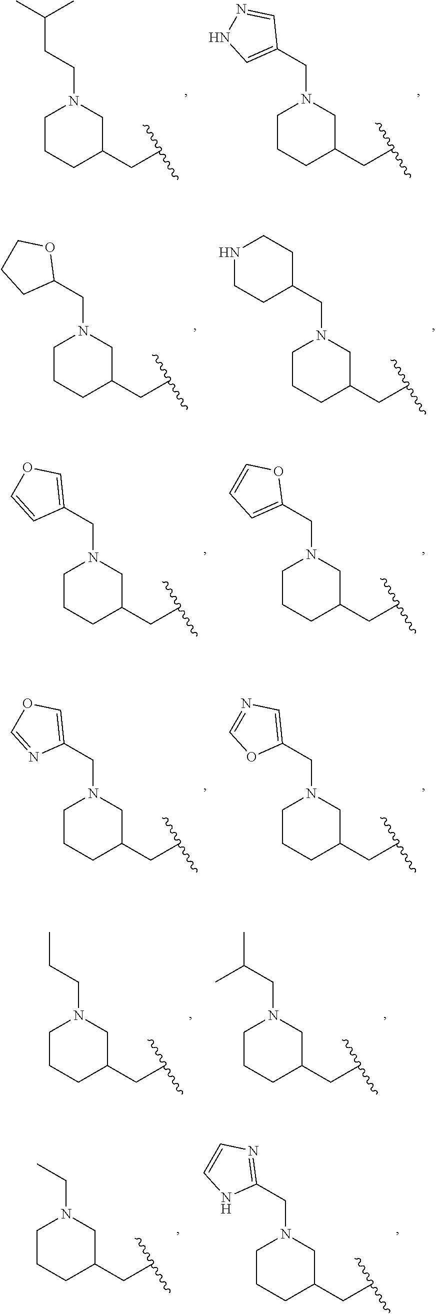

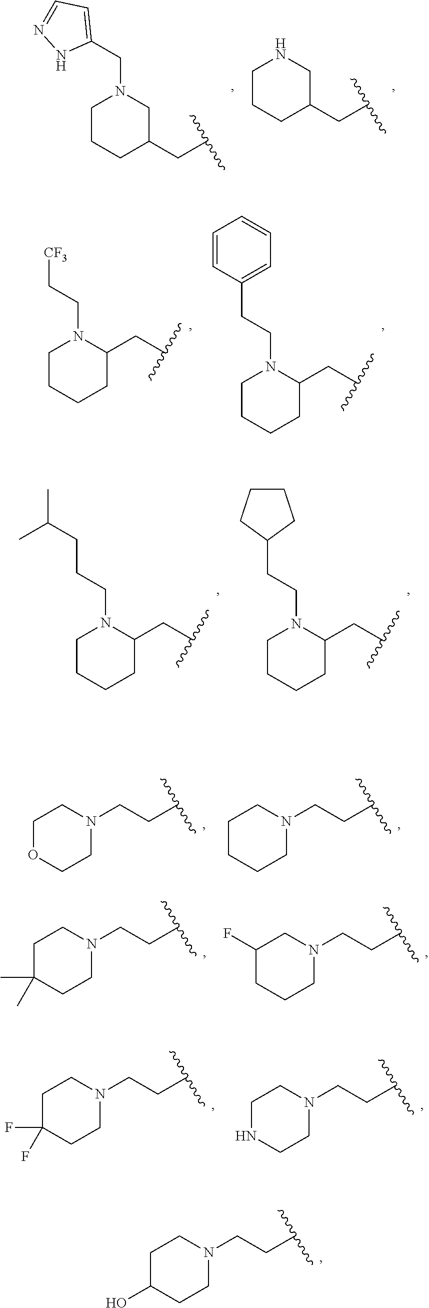

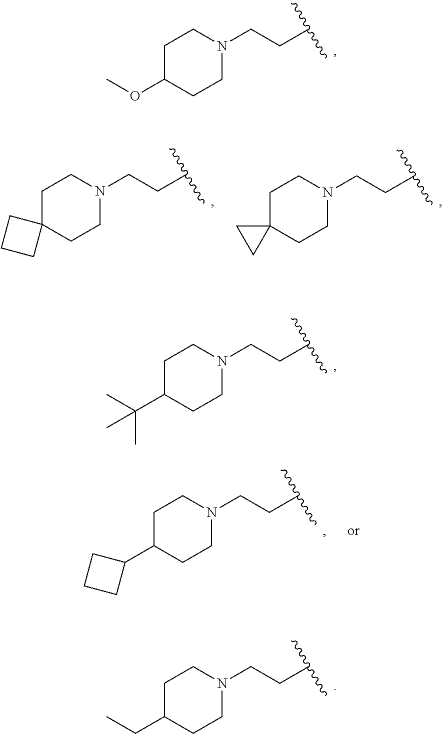 [SMILES: *CCN1CCC(OC)CC1 ; *CCN1CCC2(CCC2)CC1 ; *CCN1CCC2(CC1)CC2 ; *CCN1CCC(C(C)(C)C)CC1 ; *CCN1CCC(C2CCC2)CC1 ; *CCN1CCC(CC)CC1]

Also disclosed herein is a compound of Formula (II), or a pharmaceutically acceptable salt, solvate, or stereoisomer thereof:

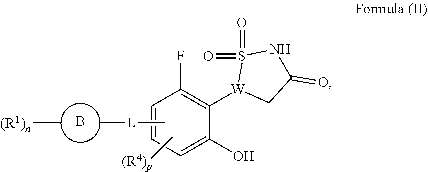

wherein:
Ring B is a cycloalkyl, heterocycloalkyl, or heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R^{1a};
or two R¹ on the same atom are taken together to form an oxo;
or two R¹ on the same carbon are taken together to form a cycloalkyl or heterocycloalkyl; each optionally substituted with one or more R;
or two R¹ on the different atoms are taken together to form a cycloalkyl, heterocycloalkyl, aryl, or heteroaryl; each optionally substituted with one or more R;
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R;
or two R^{1a} on the same atom are taken together to form an oxo;
n is 1-11;
L is —O—, —S—, —S(═O)—, —S(═O)₂—, —NR²—, [C(R³)₂]ₘ—, —NR²S(═O)₂—, —S(═O)₂NR²—, —NR²C(═O)—, —C(═O)NR²—, —O[C(R³)₂]ₘ—, —NR²[C(R³)₂]ₘ—, [C(R³)₂]ₘ—, or [C(R³)₂]ₘNR²—;
R² is hydrogen, —C(═O)R^{a}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
each R³ is independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R;
or two R³ are taken together to form a cycloalkyl or heterocycloalkyl; each optionally substituted with one or more R;
m is 1-4;
each R⁴ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
p is 0-2;
W is CR^{W} or N;
R^{W} is hydrogen, deuterium, halogen, —CN, —NO₂, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{a}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently and optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently and optionally substituted with one or more R;
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently and optionally substituted with one or more R;
or R^{c} and R^{d} are taken together with the atom to which they are attached to form a heterocycloalkyl optionally substituted with one or more R; and
each R is independently halogen, —CN, —OH, —OC₁-C₃alkyl, —OC₁-C₃haloalkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃deuteroalkyl, C₁-C₃haloalkyl, C₁-C₃hydroxyalkyl, C₁-C₃aminoalkyl, C₁-C₃heteroalkyl, or C₃-C₆cycloalkyl;
or two R on the same atom form an oxo.

In some embodiments of a compound of Formula (II), the compound is of Formula (IIa):

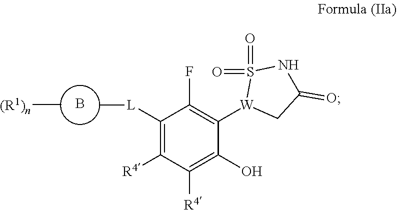

wherein R^{4′} is hydrogen or R⁴.

In some embodiments of a compound of Formula (II), the compound is of Formula (IIb):

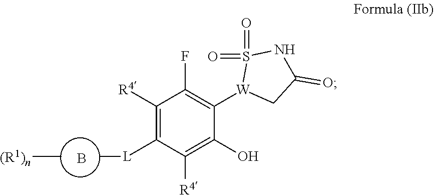

wherein R^{4′} is hydrogen or R⁴.

In some embodiments of a compound of Formula (II), the compound is of Formula (IIc):

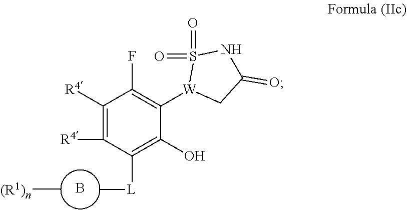

wherein R^{4′} is hydrogen or R⁴.

In some embodiments of a compound of Formula (II) or (IIa)-(IIc), Ring B is heterocycloalkyl. In some embodiments of a compound of Formula (II) or (IIa)-(IIc), Ring B is 4- to 8-membered heterocycloalkyl. In some embodiments of a compound of Formula (II) or (IIa)-(IIc), Ring B is 5- to 8-membered heterocycloalkyl. In some embodiments of a compound of Formula (II) or (IIa)-(IIc), Ring B is 5- to 6-membered heterocycloalkyl. In some embodiments of a compound of Formula (II) or (IIa)-(IIc), Ring B is 5-membered heterocycloalkyl. In some embodiments of a compound of Formula (II) or (IIa)-(IIc), Ring B is 6-membered heterocycloalkyl. In some embodiments of a compound of Formula (II) or (IIa)-(IIc), Ring B is heterocycloalkyl comprising 1 to 4 heteroatoms selected from the group consisting of O, S, and N. In some embodiments of a compound of Formula (II) or (IIa)-(IIc), Ring B is heterocycloalkyl comprising 1 to 3 heteroatoms selected from the group consisting of O, S, and N. In some embodiments of a compound of Formula (II) or (IIa)-(IIc), Ring B is heterocycloalkyl comprising 1 to 3 heteroatoms selected from the group consisting of 0 and N. In some embodiments of a compound of Formula (II) or (IIa)-(IIc), Ring B is heterocycloalkyl comprising 1 to 2 heteroatoms selected from the group consisting of 0 and N. In some embodiments of a compound of Formula (II) or (IIa)-(IIc), Ring B is heterocycloalkyl comprising 1 to 2 heteroatoms that are N. In some embodiments of a compound of Formula (II) or (IIa)-(IIc), Ring B is heterocycloalkyl comprising 1 heteroatom that is N.

In some embodiments of a compound of Formula (II) or (IIa)-(IIc), Ring B is cycloalkyl. In some embodiments of a compound of Formula (II) or (IIa)-(IIc), Ring B is 4- to 8-membered cycloalkyl. In some embodiments of a compound of Formula (II) or (IIa)-(IIc), Ring B is 5- to 8-membered cycloalkyl. In some embodiments of a compound of Formula (II) or (IIa)-(IIc), Ring B is 5- to 6-membered cycloalkyl.

In some embodiments of a compound of Formula (II) or (IIa)-(IIc), Ring B is heteroaryl. In some embodiments of a compound of Formula (II) or (IIa)-(IIc), Ring B is 5- or 6-membered heteroaryl. In some embodiments of a compound of Formula (II) or (IIa)-(IIc), Ring B is 5-membered heteroaryl. In some embodiments of a compound of Formula (II) or (IIa)-(IIc), Ring B is 6-membered heteroaryl.

In some embodiments of a compound of Formula (II) or (IIa)-(IIc), Ring B is heteroaryl comprising 1 to 4 heteroatoms selected from the group consisting of O, S, and N. In some embodiments of a compound of Formula (II) or (IIa)-(IIc), Ring B is heteroaryl comprising 1 to 3 heteroatoms selected from the group consisting of O, S, and N. In some embodiments of a compound of Formula (II) or (IIa)-(IIc), Ring B is heteroaryl comprising 1 to 3 heteroatoms selected from the group consisting of 0 and N. In some embodiments of a compound of Formula (II) or (IIa)-(IIc), Ring B is heteroaryl comprising 1 to 2 heteroatoms selected from the group consisting of 0 and N. In some embodiments of a compound of Formula (II) or (IIa)-(IIc), Ring B is heteroaryl comprising 1 to 2 heteroatoms that are N. In some embodiments of a compound of Formula (II) or (IIa)-(IIc), Ring B is heteroaryl comprising 1 heteroatom that is N.

In some embodiments of a compound of Formula (II) or (IIa)-(IIc), L is —O—, —S—, —S(═O)—, —S(═O)₂—, —NR²—, —[C(R³)₂]ₘ, —O[C(R³)₂]ₘ—, —NR²[C(R³)₂]ₘ—, —[C(R³)₂]ₘO—, or [C(R³)₂]ₘNR²—.

In some embodiments of a compound of Formula (II) or (IIa)-(IIc), L is —[C(R³)₂]—.

In some embodiments of a compound of Formula (II) or (IIa)-(IIc), L is —CH₂—, —CH₂CH₂—, or —CH₂CH₂CH₂—. In some embodiments of a compound of Formula (II) or (IIa)-(IIc), L is —CH₂—. In some embodiments of a compound of Formula (II) or (IIa)-(IIc), L is —CH₂CH₂—. In some embodiments of a compound of Formula (II) or (IIa)-(IIc), L is —CH₂CH₂CH₂—.

In some embodiments of a compound of Formula (II) or (IIa)-(IIc), L is —O—. In some embodiments of a compound of Formula (II) or (IIa)-(IIc), L is —S—, —S(═O)—, or —S(═O)₂—. In some embodiments of a compound of Formula (II) or (IIa)-(IIc), L is —NR²—.

In some embodiments of a compound of Formula (II) or (IIa)-(IIc), L is —NR²C(═O)— or —C(═O)NR²—. In some embodiments of a compound of Formula (II) or (IIa)-(IIc), L is —NR²S(═O)₂— or —S(═O)₂NR²—. In some embodiments of a compound of Formula (II) or (IIa)-(IIc), L is —O[C(R³)₂]ₘ—, —NR²[C(R³)₂]ₘ—, —[C(R³)₂]O—, or —[C(R³)₂]ₘNR²—. In some embodiments of a compound of Formula (II) or (IIa)-(IIc), L is —O[C(R³)₂]ₘ— or —NR²[C(R³)₂]ₘ—. In some embodiments of a compound of Formula (II) or (IIa)-(IIc), L is —[C(R³)₂]O— or —[C(R³)₂]ₘNR²—.

In some embodiments of a compound of Formula (II) or (IIa)-(IIc), each R³ is independently hydrogen, deuterium, halogen, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; or two R³ are taken together to form a cycloalkyl. In some embodiments of a compound of Formula (II) or (IIa)-(IIc), each R³ is independently hydrogen, deuterium, halogen, —OH, C₁-C₆alkyl, or C₁-C₆haloalkyl; or two R³ are taken together to form a cycloalkyl. In some embodiments of a compound of Formula (II) or (IIa)-(IIc), each R³ is independently hydrogen, halogen, —OH, or C₁-C₆alkyl; or two R³ are taken together to form a cycloalkyl. In some embodiments of a compound of Formula (II) or (IIa)-(IIc), two R³ are taken together to form a cycloalkyl or heterocycloalkyl; each optionally substituted with one or more R. In some embodiments of a compound of Formula (II) or (IIa)-(IIc), two R³ are taken together to form a cycloalkyl optionally substituted with one or more R.

In some embodiments of a compound of Formula (II) or (IIa)-(IIc), each R³ is independently hydrogen, deuterium, halogen, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl. In some embodiments of a compound of Formula (II) or (IIa)-(IIc), each R³ is independently hydrogen, deuterium, halogen, —OH, C₁-C₆alkyl, or C₁-C₆haloalkyl. In some embodiments of a compound of Formula (II) or (IIa)-(IIc), each R³ is independently hydrogen, C₁-C₆alkyl, or C₁-C₆haloalkyl. In some embodiments of a compound of Formula (II) or (IIa)-(IIc), each R³ is independently hydrogen or C₁-C₆alkyl. In some embodiments of a compound of Formula (II) or (IIa)-(IIc), each R³ is hydrogen.

In some embodiments of a compound of Formula (II) or (IIa)-(IIc), m is 1-3. In some embodiments of a compound of Formula (II) or (IIa)-(IIc), m is 1 or 2. In some embodiments of a compound of Formula (II) or (IIa)-(IIc), m is 1. In some embodiments of a compound of Formula (II) or (IIa)-(IIc), m is 2.

In some embodiments of a compound of Formula (II) or (IIa)-(IIc), R² is hydrogen, C₁-C₆alkyl, C₁-C₆haloalkyl, C₁-C₆deuteroalkyl, cycloalkyl, or heterocycloalkyl. In some embodiments of a compound of Formula (II) or (IIa)-(IIc), R² is hydrogen, C₁-C₆alkyl, or C₁-C₆haloalkyl. In some embodiments of a compound of Formula (II) or (IIa)-(IIc), R² is hydrogen or C₁-C₆alkyl.

In some embodiments of a compound of Formula (II) or (IIa)-(IIc), each R⁴ is independently deuterium, halogen, C₁-C₆alkyl, or C₁-C₆haloalkyl. In some embodiments of a compound of Formula (II) or (IIa)-(IIc), each R⁴ is independently deuterium or halogen. In some embodiments of a compound of Formula (II) or (IIa)-(IIc), each R⁴ is independently halogen. In some embodiments of a compound of Formula (II) or (IIa)-(IIc), each R⁴ is independently —OR^{a} or C₁-C₆alkyl.

In some embodiments of a compound of Formula (II) or (IIa)-(IIc), p is 0 or 1. In some embodiments of a compound of Formula (II) or (IIa)-(IIc), p is 0. In some embodiments of a compound of Formula (II) or (IIa)-(IIc), p is 1.

In some embodiments of a compound of Formula (II) or (IIa)-(IIc), W is N. In some embodiments of a compound of Formula (II) or (IIa)-(IIc), W is CR^{W}.

In some embodiments of a compound of Formula (II) or (IIa)-(IIc), R^{W} is hydrogen, deuterium, halogen, or C₁-C₆alkyl. In some embodiments of a compound of Formula (II) or (IIa)-(IIc), R^{W} is hydrogen or C₁-C₆alkyl. In some embodiments of a compound of Formula (II) or (IIa)-(IIc), R^{W} is hydrogen. In some embodiments of a compound of Formula (II) or (IIa)-(IIc), R^{W} is C₁-C₆alkyl. In some embodiments of a compound of Formula (II) or (IIa)-(IIc), each R¹ is independently deuterium, halogen, —CN, —OH, —OR^{a}, —S(═O)₂R^{a}, —S(═O)₂NR^{c}R^{d}, —NR^{c}R^{d}, —C(═O)R^{a}, —C(═O)OR^{a}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R^{1a}.

In some embodiments of a compound of Formula (II) or (IIa)-(IIc), each R¹ is independently deuterium, halogen, —CN, —OH, —OR^{a}, —S(═O)₂R^{a}, —S(═O)₂NR^{c}R^{d}, —NR^{c}R^{d}, —C(═O)R^{a}, —C(═O)OR^{a}, —C(═O)NR^{c}R^{d}, C₁-C₆alkyl, C₁-C₆haloalkyl, C₁-C₆deuteroalkyl, cycloalkyl, or heterocycloalkyl; wherein each alkyl, cycloalkyl, and heterocycloalkyl is independently and optionally substituted with one or more R^{1a}. In some embodiments of a compound of Formula (II) or (IIa)-(IIc), each R¹ is independently deuterium, halogen, —CN, —OH, —OR^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl. In some embodiments of a compound of Formula (II) or (IIa)-(IIc), each R¹ is independently deuterium, halogen, —S(═O)₂R^{a}, —S(═O)₂NR^{c}R^{d}, —C(═O)R^{a}, —C(═O)OR^{a}, —C(═O)NR^{c}R^{d}, C₁-C₆alkyl, C₁-C₆haloalkyl, C₁-C₆deuteroalkyl. In some embodiments of a compound of Formula (II) or (IIa)-(IIc), each R¹ is independently C₁-C₆alkyl or C₁-C₆haloalkyl. In some embodiments of a compound of Formula (II) or (IIa)-(IIc), each R¹ is independently C₁-C₆alkyl. In some embodiments of a compound of Formula (II) or (IIa)-(IIc), two R¹ on the same atom are taken together to form an oxo. In some embodiments of a compound of Formula (II) or (IIa)-(IIc), two R¹ on the same carbon are taken together to form a cycloalkyl or heterocycloalkyl; each optionally substituted with one or more R. In some embodiments of a compound of Formula (II) or (IIa)-(IIc), two R¹ on the different atoms are taken together to form a cycloalkyl, heterocycloalkyl, aryl, or heteroaryl; each optionally substituted with one or more R. In some embodiments of a compound of Formula (II) or (IIa)-(IIc), n is 1-8. In some embodiments of a compound of Formula (II) or (IIa)-(IIc), n is 1-7. In some embodiments of a compound of Formula (II) or (IIa)-(IIc), n is 1-6. In some embodiments of a compound of Formula (II) or (IIa)-(IIc), n is 1-5. In some embodiments of a compound of Formula (II) or (IIa)-(IIc), n is 1-4. In some embodiments of a compound of Formula (II) or (IIa)-(IIc), n is 1-3. In some embodiments of a compound of Formula (II) or (IIa)-(IIc), n is 1 or 2. In some embodiments of a compound of Formula (II) or (IIa)-(IIc), n is 1. In some embodiments of a compound of Formula (II) or (IIa)-(IIc), n is 2. In some embodiments of a compound of Formula (II) or (IIa)-(IIc), n is 3. In some embodiments of a compound of Formula (II) or (IIa)-(IIc), n is 4. In some embodiments of a compound of Formula (II) or (IIa)-(IIc), n is 5. In some embodiments of a compound of Formula (II) or (IIa)-(IIc), n is 6.

In some embodiments of a compound disclosed herein, each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R. In some embodiments of a compound disclosed herein, each R^{a} is independently C₁-C₆alkyl, C₁-C₆haloalkyl, C₁-C₆deuteroalkyl, cycloalkyl, or heterocycloalkyl; wherein each alkyl, cycloalkyl, and heterocycloalkyl is independently and optionally substituted with one or more R. In some embodiments of a compound disclosed herein, each R^{a} is independently C₁-C₆alkyl, C₁-C₆haloalkyl, or cycloalkyl; wherein each alkyl and cycloalkyl is independently and optionally substituted with one or more R. In some embodiments of a compound disclosed herein, each R^{a} is independently C₁-C₆alkyl or cycloalkyl; wherein each alkyl and cycloalkyl is independently and optionally substituted with one or more R. In some embodiments of a compound disclosed herein, each R^{a} is independently C₁-C₆alkyl or C₁-C₆haloalkyl; wherein each alkyl is independently and optionally substituted with one or more R.

In some embodiments of a compound disclosed herein, each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R. In some embodiments of a compound disclosed herein, each R^{b} is independently hydrogen, C₁-C₆alkyl, C₁-C₆haloalkyl, C₁-C₆deuteroalkyl, cycloalkyl, or heterocycloalkyl; wherein each alkyl, cycloalkyl, and heterocycloalkyl is independently and optionally substituted with one or more R. In some embodiments of a compound disclosed herein, each R^{b} is independently hydrogen, C₁-C₆alkyl, C₁-C₆haloalkyl, or cycloalkyl; wherein each alkyl and cycloalkyl is independently and optionally substituted with one or more R. In some embodiments of a compound disclosed herein, each R^{b} is independently hydrogen, C₁-C₆alkyl, or cycloalkyl; wherein each alkyl and cycloalkyl is independently and optionally substituted with one or more R. In some embodiments of a compound disclosed herein, each R^{b} is independently hydrogen, C₁-C₆alkyl, or C₁-C₆haloalkyl; wherein each alkyl is independently and optionally substituted with one or more R. In some embodiments of a compound disclosed herein, each R^{b} is independently hydrogen or C₁-C₆alkyl independently and optionally substituted with one or more R.

In some embodiments of a compound disclosed herein, each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R. In some embodiments of a compound disclosed herein, each R^{c} and R^{d} are independently hydrogen, C₁-C₆alkyl, C₁-C₆haloalkyl, C₁-C₆deuteroalkyl, cycloalkyl, or heterocycloalkyl; wherein each alkyl, cycloalkyl, and heterocycloalkyl is independently and optionally substituted with one or more R. In some embodiments of a compound disclosed herein, each R^{c} and R^{d} are independently hydrogen, C₁-C₆alkyl, C₁-C₆haloalkyl, or cycloalkyl; wherein each alkyl and cycloalkyl is independently and optionally substituted with one or more R. In some embodiments of a compound disclosed herein, each R^{c} and R^{d} are independently hydrogen, C₁-C₆alkyl, or cycloalkyl; wherein each alkyl and cycloalkyl is independently and optionally substituted with one or more R. In some embodiments of a compound disclosed herein, each R^{c} and R^{d} are independently hydrogen, C₁-C₆alkyl, or C₁-C₆haloalkyl; wherein each alkyl is independently and optionally substituted with one or more R. In some embodiments of a compound disclosed herein, each R^{c} and R^{d} are independently hydrogen or C₁-C₆alkyl independently and optionally substituted with one or more R. In some embodiments of a compound disclosed herein, R^{c} is cycloalkyl and R^{d} hydrogen.

In some embodiments of a compound disclosed herein, R^{c} and R^{d} are taken together with the atom to which they are attached to form a heterocycloalkyl optionally substituted with one or more R. In some embodiments of a compound disclosed herein, R^{c} and R^{d} are taken together with the atom to which they are attached to form a heterocycloalkyl.

In some embodiments of a compound disclosed herein, each R is independently halogen, —CN, —OH, —OC₁-C₃alkyl, —OC₁-C₃haloalkyl, NH₂, —NHC₁-C₃alkyl, —N(C₁-C₃alkyl)₂, C₁-C₃alkyl, C₁-C₃deuteroalkyl, C₁-C₃haloalkyl, C₁-C₃hydroxyalkyl, C₁-C₃aminoalkyl, C₁-C₃heteroalkyl, or C₃-C₆cycloalkyl. In some embodiments of a compound disclosed herein, each R is independently halogen, —CN, —OH, —OC₁-C₃alkyl, —OC₁-C₃haloalkyl, NH₂, C₁-C₃alkyl, C₁-C₃deuteroalkyl, or C₁-C₃haloalkyl. In some embodiments of a compound disclosed herein, each R is independently halogen, —CN, —OH, —OC₁-C₃alkyl, NH₂, C₁-C₃alkyl, or C₁-C₃haloalkyl. In some embodiments of a compound disclosed herein, each R is independently halogen, C₁-C₃alkyl, or C₁-C₃haloalkyl.

Any combination of the groups described above for the various variables is contemplated herein. Throughout the specification, groups and substituents thereof are chosen by one skilled in the field to provide stable moieties and compounds.

In some embodiments, the compound disclosed herein is a compound selected from Table 1, or a pharmaceutically acceptable salt, solvate, or stereoisomer thereof.

TABLE 1

| Example | Structure |
|---|---|
| 1 | 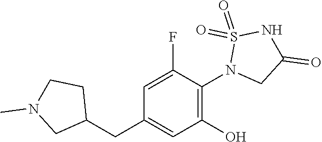 [SMILES: CN1CCC(Cc2cc(O)c(N3CC(=O)NS3(=O)=O)c(F)c2)C1] |
| 2 | 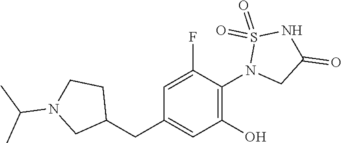 [SMILES: CC(C)N1CCC(Cc2cc(O)c(N3CC(=O)NS3(=O)=O)c(F)c2)C1] |
| 3 | 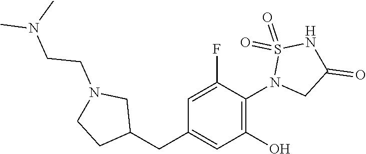 [SMILES: CN(C)CCN1CCC(Cc2cc(O)c(N3CC(=O)NS3(=O)=O)c(F)c2)C1] |
| 4 | 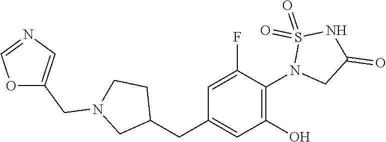 [SMILES: O=C1CN(c2c(O)cc(CC3CCN(Cc4cnco4)C3)cc2F)S(=O)(=O)N1] |
| 5 | 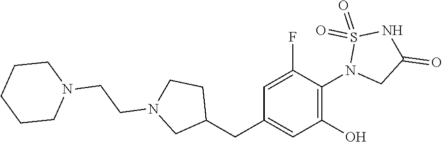 [SMILES: O=C1CN(c2c(O)cc(CC3CCN(CCN4CCCCC4)C3)cc2F)S(=O)(=O)N1] |
| 6 | 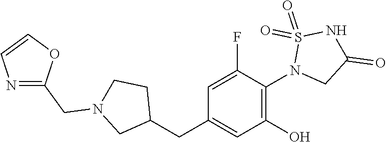 [SMILES: O=C1CN(c2c(O)cc(CC3CCN(Cc4ncco4)C3)cc2F)S(=O)(=O)N1] |
| 7 | 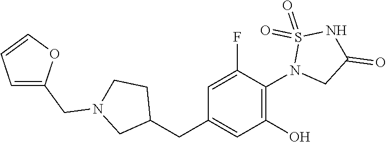 [SMILES: O=C1CN(c2c(O)cc(CC3CCN(Cc4ccco4)C3)cc2F)S(=O)(=O)N1] |
| 8 | 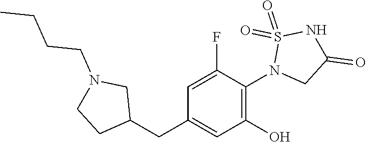 [SMILES: CCCCN1CCC(Cc2cc(O)c(N3CC(=O)NS3(=O)=O)c(F)c2)C1] |
| 9 | 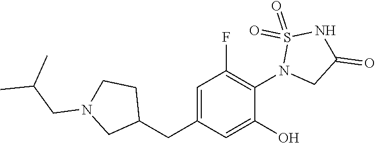 [SMILES: CC(C)CN1CCC(Cc2cc(O)c(N3CC(=O)NS3(=O)=O)c(F)c2)C1] |
| 10 | 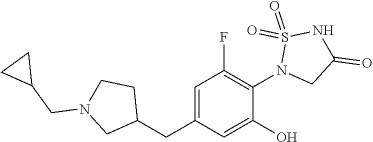 [SMILES: O=C1CN(c2c(O)cc(CC3CCN(CC4CC4)C3)cc2F)S(=O)(=O)N1] |
| 11 | 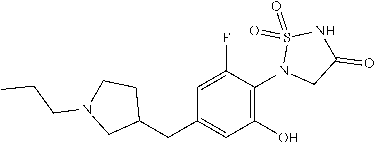 [SMILES: CCCN1CCC(Cc2cc(O)c(N3CC(=O)NS3(=O)=O)c(F)c2)C1] |
| 12 | 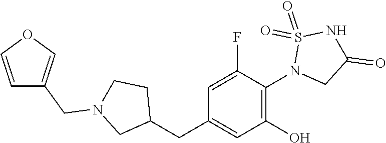 [SMILES: O=C1CN(c2c(O)cc(CC3CCN(Cc4ccoc4)C3)cc2F)S(=O)(=O)N1] |
| 13 | 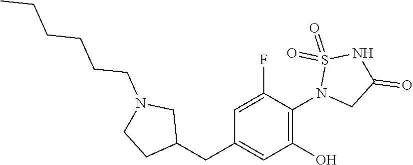 [SMILES: CCCCCCN1CCC(Cc2cc(O)c(N3CC(=O)NS3(=O)=O)c(F)c2)C1] |
| 14 | 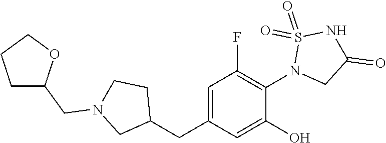 [SMILES: O=C1CN(c2c(O)cc(CC3CCN(CC4CCCO4)C3)cc2F)S(=O)(=O)N1] |
| 15 | 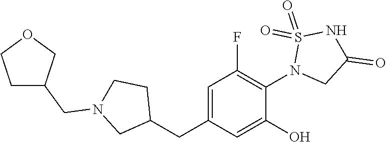 [SMILES: O=C1CN(c2c(O)cc(CC3CCN(CC4CCOC4)C3)cc2F)S(=O)(=O)N1] |
| 16 | 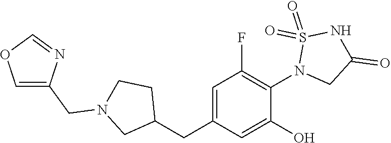 [SMILES: O=C1CN(c2c(O)cc(CC3CCN(Cc4cocn4)C3)cc2F)S(=O)(=O)N1] |
| 17 | 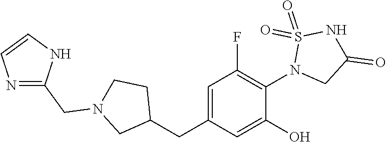 [SMILES: O=C1CN(c2c(O)cc(CC3CCN(Cc4ncc[nH]4)C3)cc2F)S(=O)(=O)N1] |
| 18 | 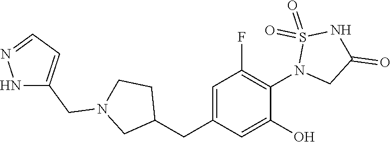 [SMILES: O=C1CN(c2c(O)cc(CC3CCN(Cc4ccn[nH]4)C3)cc2F)S(=O)(=O)N1] |
| 19 | 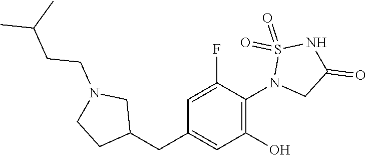 [SMILES: CC(C)CCN1CCC(Cc2cc(O)c(N3CC(=O)NS3(=O)=O)c(F)c2)C1] |
| 20 | 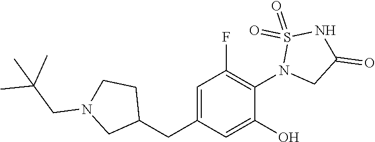 [SMILES: CC(C)(C)CN1CCC(Cc2cc(O)c(N3CC(=O)NS3(=O)=O)c(F)c2)C1] |
| 21 | 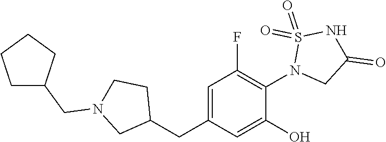 [SMILES: O=C1CN(c2c(O)cc(CC3CCN(CC4CCCC4)C3)cc2F)S(=O)(=O)N1] |
| 22 | 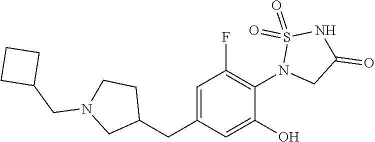 [SMILES: O=C1CN(c2c(O)cc(CC3CCN(CC4CCC4)C3)cc2F)S(=O)(=O)N1] |
| 23 | 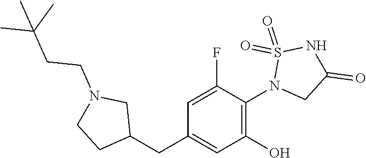 [SMILES: CC(C)(C)CCN1CCC(Cc2cc(O)c(N3CC(=O)NS3(=O)=O)c(F)c2)C1] |
| 24 | 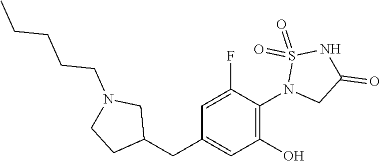 [SMILES: CCCCCN1CCC(Cc2cc(O)c(N3CC(=O)NS3(=O)=O)c(F)c2)C1] |
| 25* | 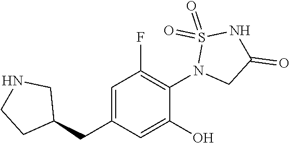 [SMILES: O=C1CN(c2c(O)cc(C[C@H]3CCNC3)cc2F)S(=O)(=O)N1] |
| 26* | 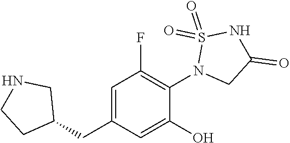 [SMILES: O=C1CN(c2c(O)cc(C[C@@H]3CCNC3)cc2F)S(=O)(=O)N1] |
| 27 | 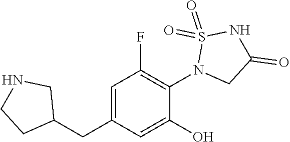 [SMILES: O=C1CN(c2c(O)cc(CC3CCNC3)cc2F)S(=O)(=O)N1] |
| 28 | 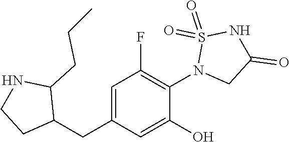 [SMILES: CCCC1NCCC1Cc1cc(O)c(N2CC(=O)NS2(=O)=O)c(F)c1] |
| 29 | 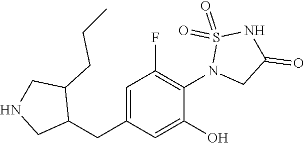 [SMILES: CCCC1CNCC1Cc1cc(O)c(N2CC(=O)NS2(=O)=O)c(F)c1] |
| 30 | 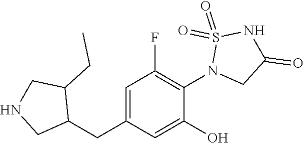 [SMILES: CCC1CNCC1Cc1cc(O)c(N2CC(=O)NS2(=O)=O)c(F)c1] |
| 31 | 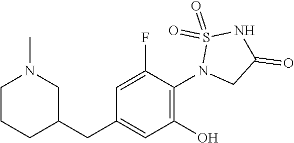 [SMILES: CN1CCCC(Cc2cc(O)c(N3CC(=O)NS3(=O)=O)c(F)c2)C1] |
| 32 | 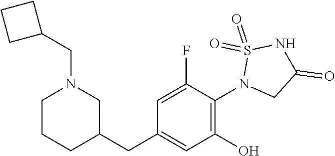 [SMILES: O=C1CN(c2c(O)cc(CC3CCCN(CC4CCC4)C3)cc2F)S(=O)(=O)N1] |
| 33 | 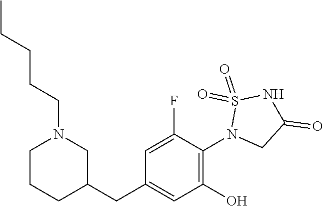 [SMILES: CCCCCN1CCCC(Cc2cc(O)c(N3CC(=O)NS3(=O)=O)c(F)c2)C1] |
| 34 | 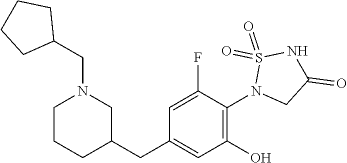 [SMILES: O=C1CN(c2c(O)cc(CC3CCCN(CC4CCCC4)C3)cc2F)S(=O)(=O)N1] |
| 35 | 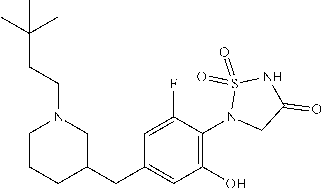 [SMILES: CC(C)(C)CCN1CCCC(Cc2cc(O)c(N3CC(=O)NS3(=O)=O)c(F)c2)C1] |
| 36 | 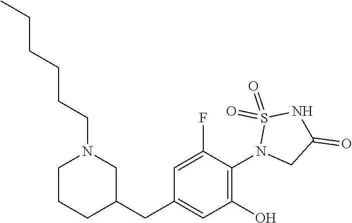 [SMILES: CCCCCCN1CCCC(Cc2cc(O)c(N3CC(=O)NS3(=O)=O)c(F)c2)C1] |
| 37 | 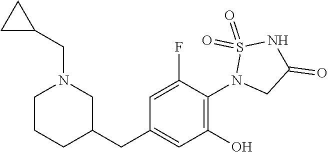 [SMILES: O=C1CN(c2c(O)cc(CC3CCCN(CC4CC4)C3)cc2F)S(=O)(=O)N1] |
| 38 | 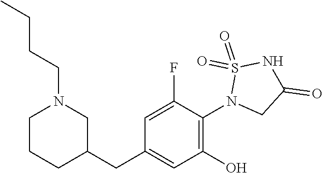 [SMILES: CCCCN1CCCC(Cc2cc(O)c(N3CC(=O)NS3(=O)=O)c(F)c2)C1] |
| 39 | 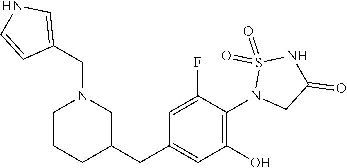 [SMILES: O=C1CN(c2c(O)cc(CC3CCCN(Cc4cc[nH]c4)C3)cc2F)S(=O)(=O)N1] |
| 40 | 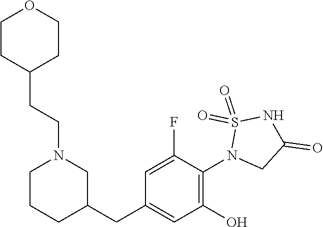 [SMILES: O=C1CN(c2c(O)cc(CC3CCCN(CCC4CCOCC4)C3)cc2F)S(=O)(=O)N1] |
| 41 | 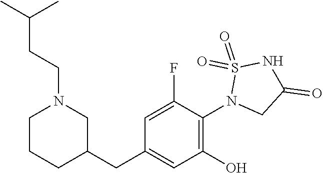 [SMILES: CC(C)CCN1CCCC(Cc2cc(O)c(N3CC(=O)NS3(=O)=O)c(F)c2)C1] |
| 42 | 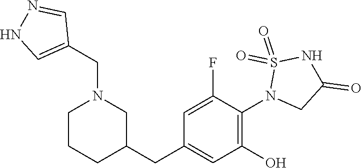 [SMILES: O=C1CN(c2c(O)cc(CC3CCCN(Cc4cn[nH]c4)C3)cc2F)S(=O)(=O)N1] |
| 43 | 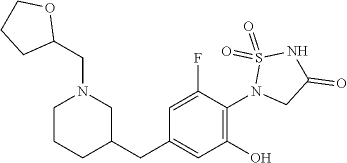 [SMILES: O=C1CN(c2c(O)cc(CC3CCCN(CC4CCCO4)C3)cc2F)S(=O)(=O)N1] |
| 44 | 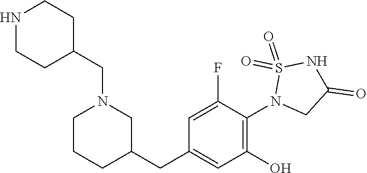 [SMILES: O=C1CN(c2c(O)cc(CC3CCCN(CC4CCNCC4)C3)cc2F)S(=O)(=O)N1] |
| 45 | 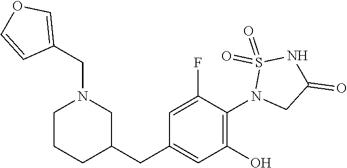 [SMILES: O=C1CN(c2c(O)cc(CC3CCCN(Cc4ccoc4)C3)cc2F)S(=O)(=O)N1] |
| 46 | 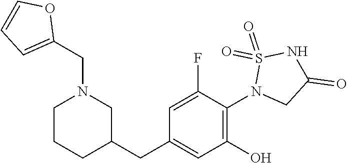 [SMILES: O=C1CN(c2c(O)cc(CC3CCCN(Cc4ccco4)C3)cc2F)S(=O)(=O)N1] |
| 47 | 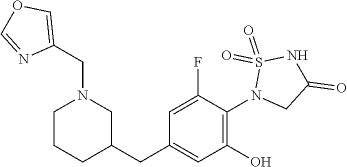 [SMILES: O=C1CN(c2c(O)cc(CC3CCCN(Cc4cocn4)C3)cc2F)S(=O)(=O)N1] |
| 48 | 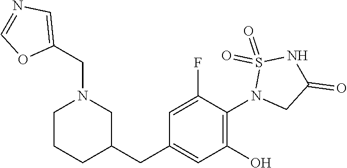 [SMILES: O=C1CN(c2c(O)cc(CC3CCCN(Cc4cnco4)C3)cc2F)S(=O)(=O)N1] |
| 49 | 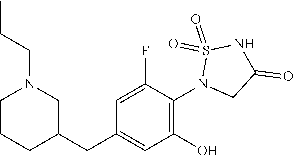 [SMILES: CCCN1CCCC(Cc2cc(O)c(N3CC(=O)NS3(=O)=O)c(F)c2)C1] |
| 50 | 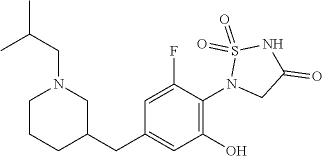 [SMILES: CC(C)CN1CCCC(Cc2cc(O)c(N3CC(=O)NS3(=O)=O)c(F)c2)C1] |
| 51 | 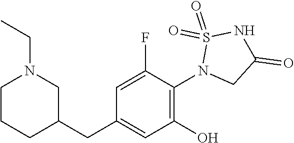 [SMILES: CCN1CCCC(Cc2cc(O)c(N3CC(=O)NS3(=O)=O)c(F)c2)C1] |
| 52 | 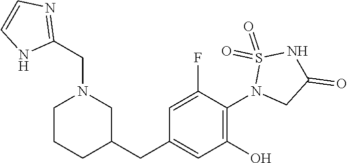 [SMILES: O=C1CN(c2c(O)cc(CC3CCCN(Cc4ncc[nH]4)C3)cc2F)S(=O)(=O)N1] |
| 53 | 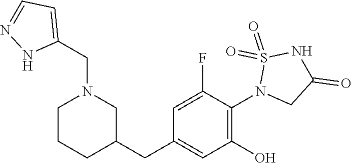 [SMILES: O=C1CN(c2c(O)cc(CC3CCCN(Cc4ccn[nH]4)C3)cc2F)S(=O)(=O)N1] |
| 54 | 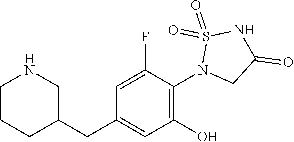 [SMILES: O=C1CN(c2c(O)cc(CC3CCCNC3)cc2F)S(=O)(=O)N1] |
| 55 | 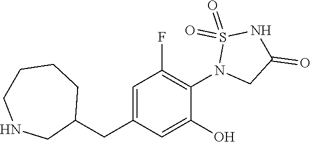 [SMILES: O=C1CN(c2c(O)cc(CC3CCCCNC3)cc2F)S(=O)(=O)N1] |
| 56 | 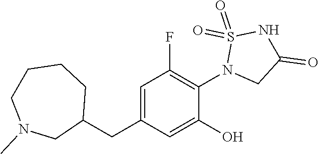 [SMILES: CN1CCCCC(Cc2cc(O)c(N3CC(=O)NS3(=O)=O)c(F)c2)C1] |
| 57 | 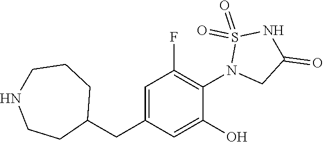 [SMILES: O=C1CN(c2c(O)cc(CC3CCCNCC3)cc2F)S(=O)(=O)N1] |
| 58* | 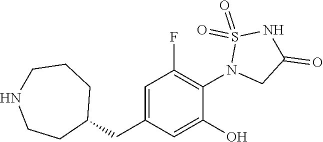 [SMILES: O=C1CN(c2c(O)cc(C[C@H]3CCCNCC3)cc2F)S(=O)(=O)N1] |
| 59* | 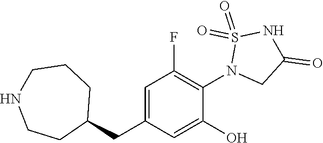 [SMILES: O=C1CN(c2c(O)cc(C[C@@H]3CCCNCC3)cc2F)S(=O)(=O)N1] |
| 60 | 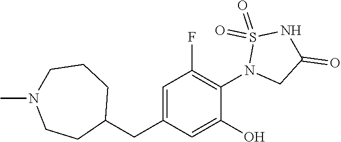 [SMILES: CN1CCCC(Cc2cc(O)c(N3CC(=O)NS3(=O)=O)c(F)c2)CC1] |
| 61 | 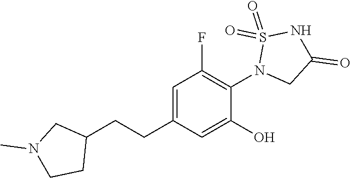 [SMILES: CN1CCC(CCc2cc(O)c(N3CC(=O)NS3(=O)=O)c(F)c2)C1] |
| 62 | 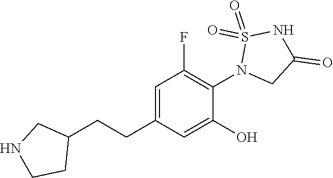 [SMILES: O=C1CN(c2c(O)cc(CCC3CCNC3)cc2F)S(=O)(=O)N1] |
| 63 | 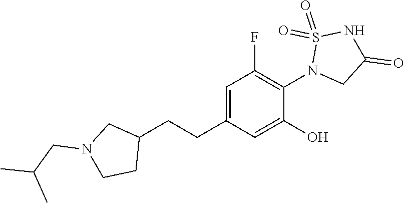 [SMILES: CC(C)CN1CCC(CCc2cc(O)c(N3CC(=O)NS3(=O)=O)c(F)c2)C1] |
| 64 | 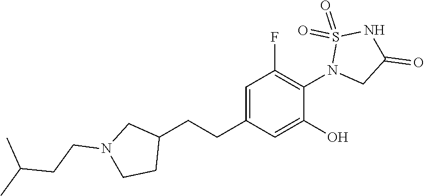 [SMILES: CC(C)CCN1CCC(CCc2cc(O)c(N3CC(=O)NS3(=O)=O)c(F)c2)C1] |
| 65 | 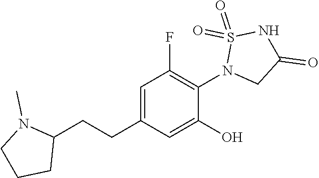 [SMILES: CN1CCCC1CCc1cc(O)c(N2CC(=O)NS2(=O)=O)c(F)c1] |
| 66 | 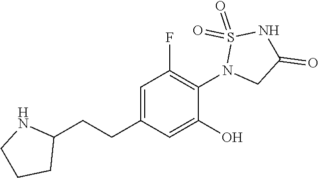 [SMILES: O=C1CN(c2c(O)cc(CCC3CCCN3)cc2F)S(=O)(=O)N1] |
| 67 | 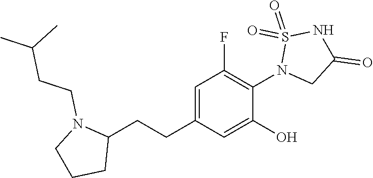 [SMILES: CC(C)CCN1CCCC1CCc1cc(O)c(N2CC(=O)NS2(=O)=O)c(F)c1] |
| 68 | 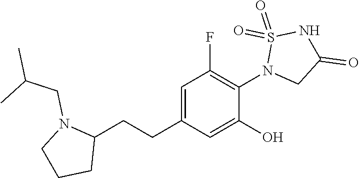 [SMILES: CC(C)CN1CCCC1CCc1cc(O)c(N2CC(=O)NS2(=O)=O)c(F)c1] |
| 69 | 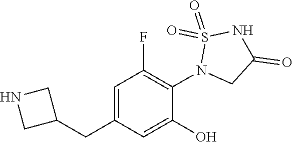 [SMILES: O=C1CN(c2c(O)cc(CC3CNC3)cc2F)S(=O)(=O)N1] |
| 70 | 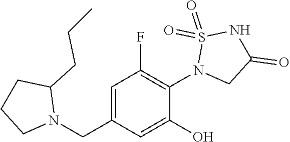 [SMILES: CCCC1CCCN1Cc1cc(O)c(N2CC(=O)NS2(=O)=O)c(F)c1] |
| 71 | 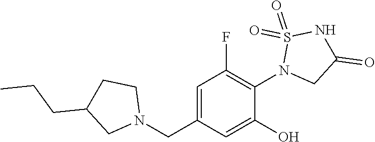 [SMILES: CCCC1CCN(Cc2cc(O)c(N3CC(=O)NS3(=O)=O)c(F)c2)C1] |
| 72 | 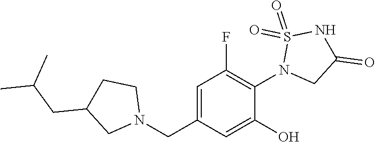 [SMILES: CC(C)CC1CCN(Cc2cc(O)c(N3CC(=O)NS3(=O)=O)c(F)c2)C1] |
| 73 | 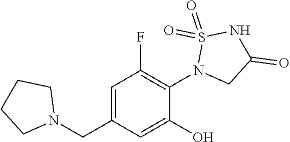 [SMILES: O=C1CN(c2c(O)cc(CN3CCCC3)cc2F)S(=O)(=O)N1] |
| 74 | 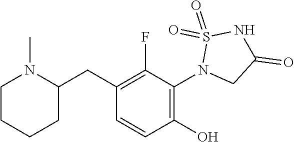 [SMILES: CN1CCCCC1Cc1ccc(O)c(N2CC(=O)NS2(=O)=O)c1F] |
| 75 | 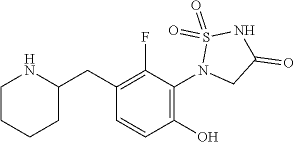 [SMILES: O=C1CN(c2c(O)ccc(CC3CCCCN3)c2F)S(=O)(=O)N1] |
| 76 | 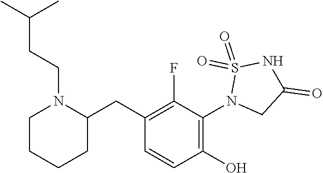 [SMILES: CC(C)CCN1CCCCC1Cc1ccc(O)c(N2CC(=O)NS2(=O)=O)c1F] |
| 77 | 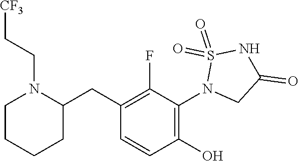 [SMILES: O=C1CN(c2c(O)ccc(CC3CCCCN3CCC(F)(F)F)c2F)S(=O)(=O)N1] |
| 78 | 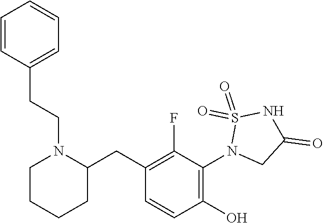 [SMILES: O=C1CN(c2c(O)ccc(CC3CCCCN3CCc3ccccc3)c2F)S(=O)(=O)N1] |
| 79 | 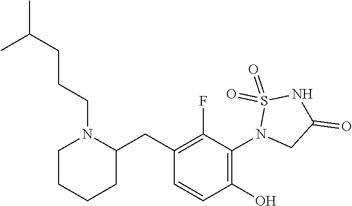 [SMILES: CC(C)CCCN1CCCCC1Cc1ccc(O)c(N2CC(=O)NS2(=O)=O)c1F] |
| 80 | 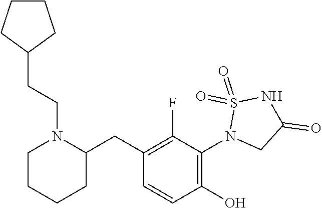 [SMILES: O=C1CN(c2c(O)ccc(CC3CCCCN3CCC3CCCC3)c2F)S(=O)(=O)N1] |
| 81* | 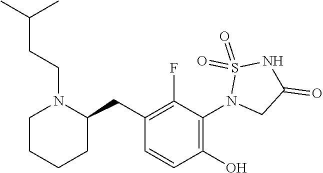 [SMILES: CC(C)CCN1CCCC[C@@H]1Cc1ccc(O)c(N2CC(=O)NS2(=O)=O)c1F] |
| 82* | 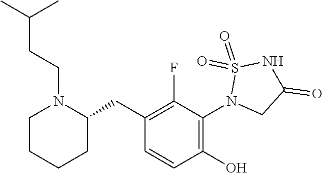 [SMILES: CC(C)CCN1CCCC[C@H]1Cc1ccc(O)c(N2CC(=O)NS2(=O)=O)c1F] |
| 83 | 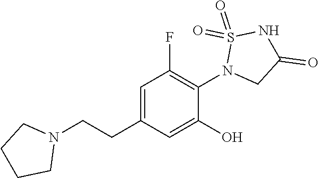 [SMILES: O=C1CN(c2c(O)cc(CCN3CCCC3)cc2F)S(=O)(=O)N1] |
| 84 | 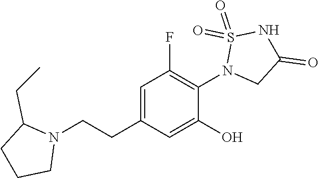 [SMILES: CCC1CCCN1CCc1cc(O)c(N2CC(=O)NS2(=O)=O)c(F)c1] |
| 85 | 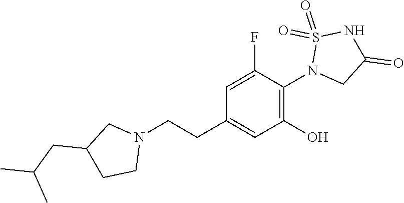 [SMILES: CC(C)CC1CCN(CCc2cc(O)c(N3CC(=O)NS3(=O)=O)c(F)c2)C1] |
| 86 | 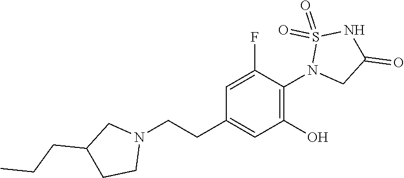 [SMILES: CCCC1CCN(CCc2cc(O)c(N3CC(=O)NS3(=O)=O)c(F)c2)C1] |
| 87 | 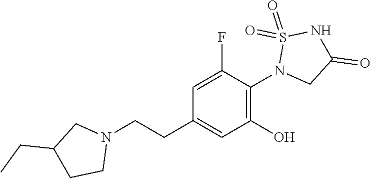 [SMILES: CCC1CCN(CCc2cc(O)c(N3CC(=O)NS3(=O)=O)c(F)c2)C1] |
| 88 | 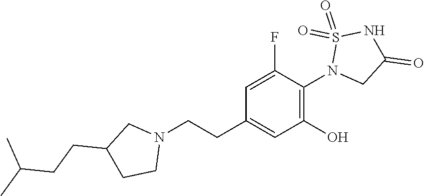 [SMILES: CC(C)CCC1CCN(CCc2cc(O)c(N3CC(=O)NS3(=O)=O)c(F)c2)C1] |
| 89 | 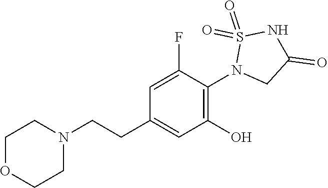 [SMILES: O=C1CN(c2c(O)cc(CCN3CCOCC3)cc2F)S(=O)(=O)N1] |
| 90 | 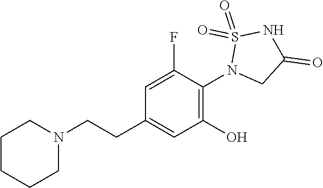 [SMILES: O=C1CN(c2c(O)cc(CCN3CCCCC3)cc2F)S(=O)(=O)N1] |
| 91 | 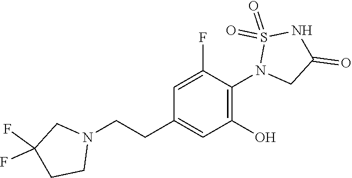 [SMILES: O=C1CN(c2c(O)cc(CCN3CCC(F)(F)C3)cc2F)S(=O)(=O)N1] |
| 92 | 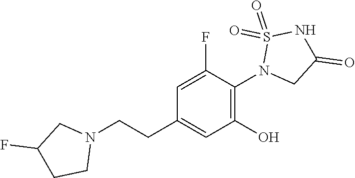 [SMILES: O=C1CN(c2c(O)cc(CCN3CCC(F)C3)cc2F)S(=O)(=O)N1] |
| 93 | 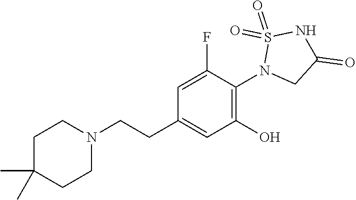 [SMILES: CC1(C)CCN(CCc2cc(O)c(N3CC(=O)NS3(=O)=O)c(F)c2)CC1] |
| 94 | 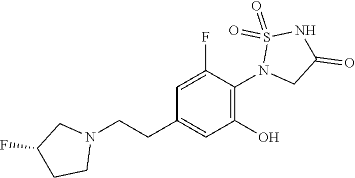 [SMILES: O=C1CN(c2c(O)cc(CCN3CC[C@H](F)C3)cc2F)S(=O)(=O)N1] |
| 95 | 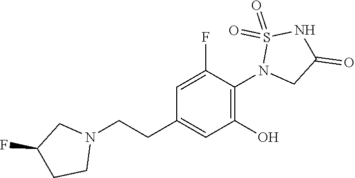 [SMILES: O=C1CN(c2c(O)cc(CCN3CC[C@@H](F)C3)cc2F)S(=O)(=O)N1] |
| 96 | 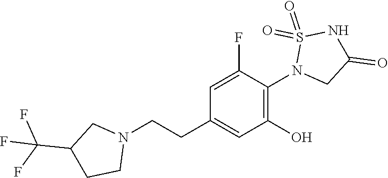 [SMILES: O=C1CN(c2c(O)cc(CCN3CCC(C(F)(F)F)C3)cc2F)S(=O)(=O)N1] |
| 97 | 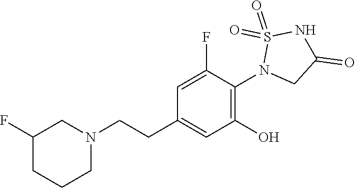 [SMILES: O=C1CN(c2c(O)cc(CCN3CCCC(F)C3)cc2F)S(=O)(=O)N1] |
| 98 | 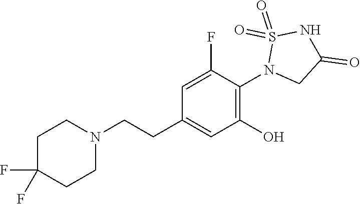 [SMILES: O=C1CN(c2c(O)cc(CCN3CCC(F)(F)CC3)cc2F)S(=O)(=O)N1] |
| 99 | 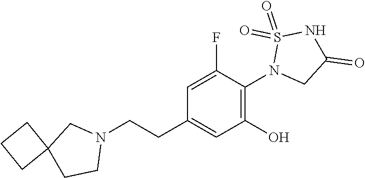 [SMILES: O=C1CN(c2c(O)cc(CCN3CCC4(CCC4)C3)cc2F)S(=O)(=O)N1] |
| 100 | 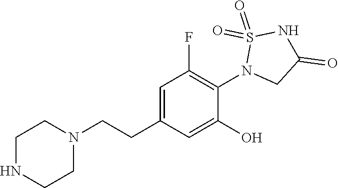 [SMILES: O=C1CN(c2c(O)cc(CCN3CCNCC3)cc2F)S(=O)(=O)N1] |
| 101 | 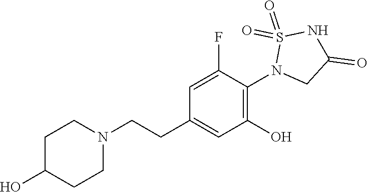 [SMILES: O=C1CN(c2c(O)cc(CCN3CCC(O)CC3)cc2F)S(=O)(=O)N1] |
| 102 | 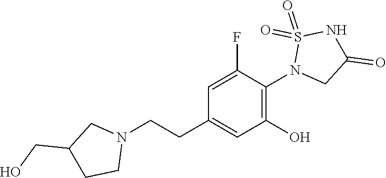 [SMILES: O=C1CN(c2c(O)cc(CCN3CCC(CO)C3)cc2F)S(=O)(=O)N1] |
| 103 | 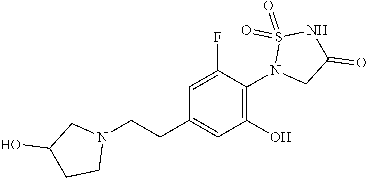 [SMILES: O=C1CN(c2c(O)cc(CCN3CCC(O)C3)cc2F)S(=O)(=O)N1] |
| 104 | 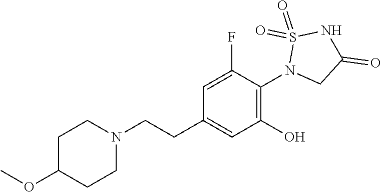 [SMILES: COC1CCN(CCc2cc(O)c(N3CC(=O)NS3(=O)=O)c(F)c2)CC1] |
| 105 | 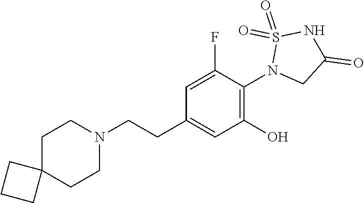 [SMILES: O=C1CN(c2c(O)cc(CCN3CCC4(CCC4)CC3)cc2F)S(=O)(=O)N1] |
| 106 | 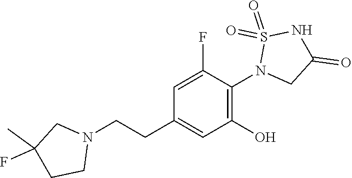 [SMILES: CC1(F)CCN(CCc2cc(O)c(N3CC(=O)NS3(=O)=O)c(F)c2)C1] |
| 107 | 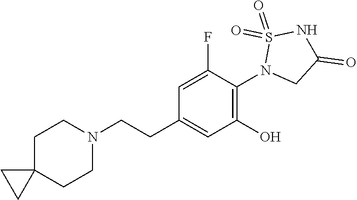 [SMILES: O=C1CN(c2c(O)cc(CCN3CCC4(CC3)CC4)cc2F)S(=O)(=O)N1] |
| 108 | 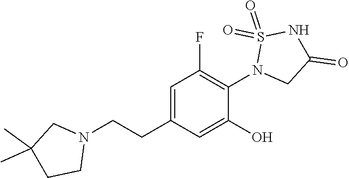 [SMILES: CC1(C)CCN(CCc2cc(O)c(N3CC(=O)NS3(=O)=O)c(F)c2)C1] |
| 109 | 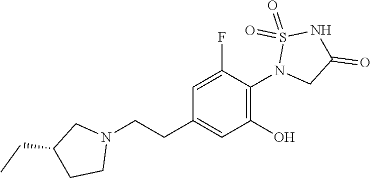 [SMILES: CC[C@H]1CCN(CCc2cc(O)c(N3CC(=O)NS3(=O)=O)c(F)c2)C1] |
| 110 | 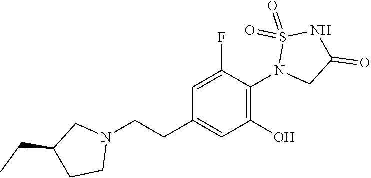 [SMILES: CC[C@@H]1CCN(CCc2cc(O)c(N3CC(=O)NS3(=O)=O)c(F)c2)C1] |
| 111 | 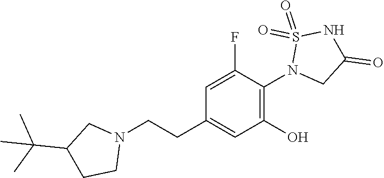 [SMILES: CC(C)(C)C1CCN(CCc2cc(O)c(N3CC(=O)NS3(=O)=O)c(F)c2)C1] |
| 112 | 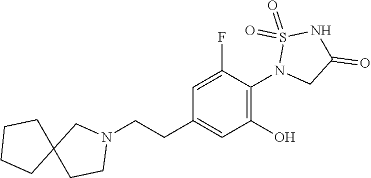 [SMILES: O=C1CN(c2c(O)cc(CCN3CCC4(CCCC4)C3)cc2F)S(=O)(=O)N1] |
| 113 | 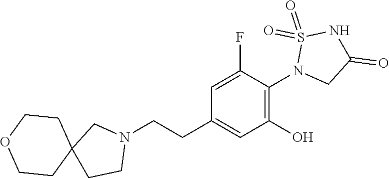 [SMILES: O=C1CN(c2c(O)cc(CCN3CCC4(CCOCC4)C3)cc2F)S(=O)(=O)N1] |
| 114 | 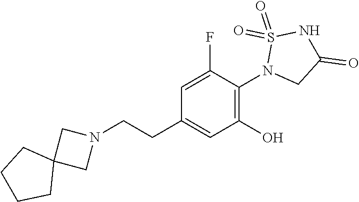 [SMILES: O=C1CN(c2c(O)cc(CCN3CC4(CCCC4)C3)cc2F)S(=O)(=O)N1] |
| 115 | 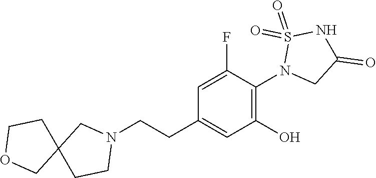 [SMILES: O=C1CN(c2c(O)cc(CCN3CCC4(CCOC4)C3)cc2F)S(=O)(=O)N1] |
| 116 | 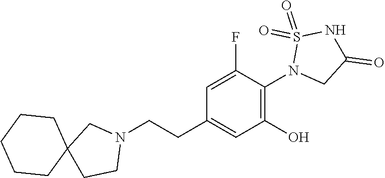 [SMILES: O=C1CN(c2c(O)cc(CCN3CCC4(CCCCC4)C3)cc2F)S(=O)(=O)N1] |
| 117 | 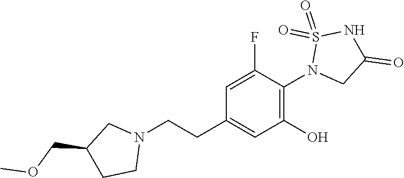 [SMILES: COC[C@@H]1CCN(CCc2cc(O)c(N3CC(=O)NS3(=O)=O)c(F)c2)C1] |
| 118 | 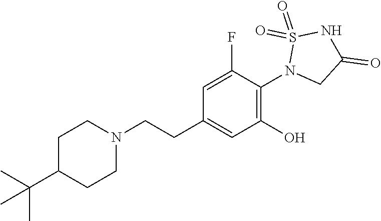 [SMILES: CC(C)(C)C1CCN(CCc2cc(O)c(N3CC(=O)NS3(=O)=O)c(F)c2)CC1] |
| 119* | 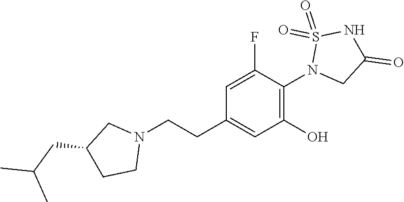 [SMILES: CC(C)C[C@H]1CCN(CCc2cc(O)c(N3CC(=O)NS3(=O)=O)c(F)c2)C1] |
| 120* | 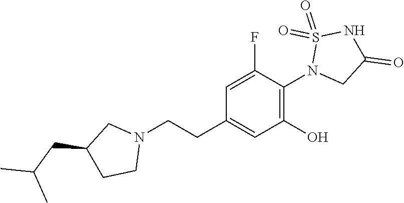 [SMILES: CC(C)C[C@@H]1CCN(CCc2cc(O)c(N3CC(=O)NS3(=O)=O)c(F)c2)C1] |
| 121 | 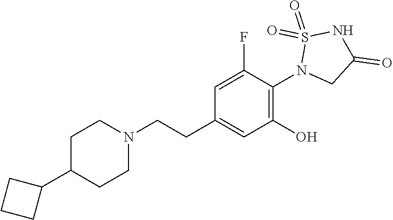 [SMILES: O=C1CN(c2c(O)cc(CCN3CCC(C4CCC4)CC3)cc2F)S(=O)(=O)N1] |
| 122* | 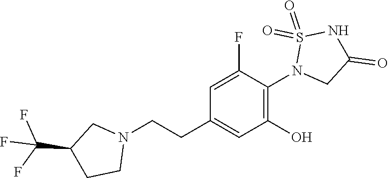 [SMILES: O=C1CN(c2c(O)cc(CCN3CC[C@@H](C(F)(F)F)C3)cc2F)S(=O)(=O)N1] |
| 123* | 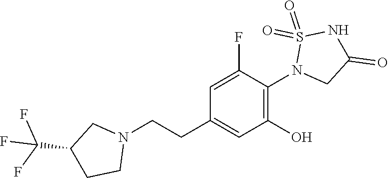 [SMILES: O=C1CN(c2c(O)cc(CCN3CC[C@H](C(F)(F)F)C3)cc2F)S(=O)(=O)N1] |
| 124 | 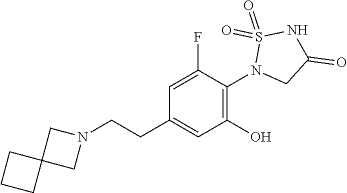 [SMILES: O=C1CN(c2c(O)cc(CCN3CC4(CCC4)C3)cc2F)S(=O)(=O)N1] |
| 125 | 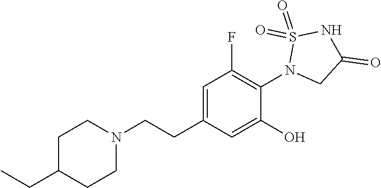 [SMILES: CCC1CCN(CCc2cc(O)c(N3CC(=O)NS3(=O)=O)c(F)c2)CC1] |
| 126 | 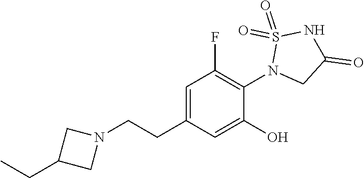 [SMILES: CCC1CN(CCc2cc(O)c(N3CC(=O)NS3(=O)=O)c(F)c2)C1] |
| 127 | 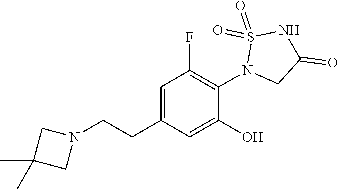 [SMILES: CC1(C)CN(CCc2cc(O)c(N3CC(=O)NS3(=O)=O)c(F)c2)C1] |
| 128 | 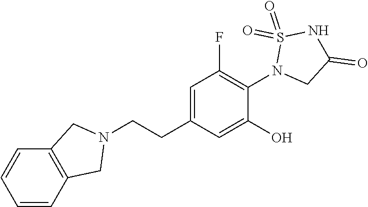 [SMILES: O=C1CN(c2c(O)cc(CCN3Cc4ccccc4C3)cc2F)S(=O)(=O)N1] |
| 129 | 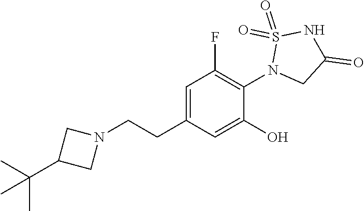 [SMILES: CC(C)(C)C1CN(CCc2cc(O)c(N3CC(=O)NS3(=O)=O)c(F)c2)C1] |
| 130 | 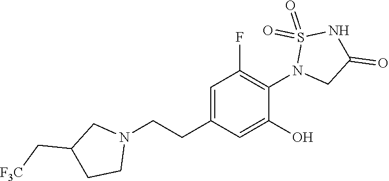 [SMILES: O=C1CN(c2c(O)cc(CCN3CCC(CC(F)(F)F)C3)cc2F)S(=O)(=O)N1] |
| 131 | 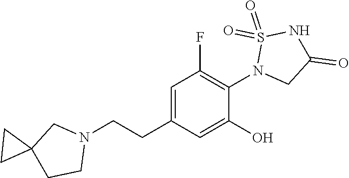 [SMILES: O=C1CN(c2c(O)cc(CCN3CCC4(CC4)C3)cc2F)S(=O)(=O)N1] |
| 132 | 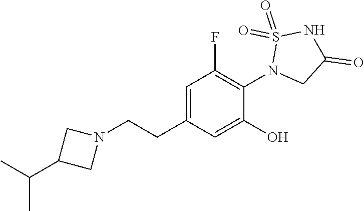 |
| 133 | 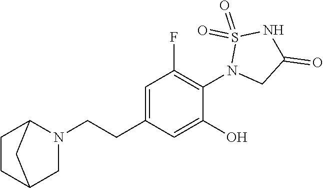 [SMILES: O=C1CN(c2c(O)cc(CCN3CC4CCC3C4)cc2F)S(=O)(=O)N1] |
| 134 | 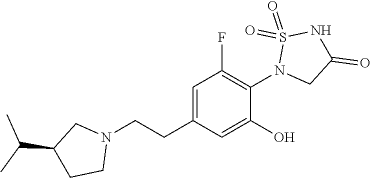 [SMILES: CC(C)[C@@H]1CCN(CCc2cc(O)c(N3CC(=O)NS3(=O)=O)c(F)c2)C1] |
| 135 | 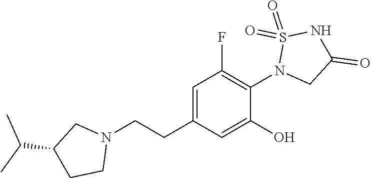 [SMILES: CC(C)[C@H]1CCN(CCc2cc(O)c(N3CC(=O)NS3(=O)=O)c(F)c2)C1] |
| 136 | 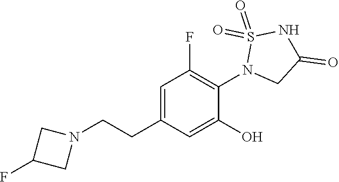 [SMILES: O=C1CN(c2c(O)cc(CCN3CC(F)C3)cc2F)S(=O)(=O)N1] |
| 137 | 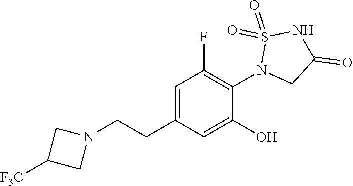 [SMILES: O=C1CN(c2c(O)cc(CCN3CC(C(F)(F)F)C3)cc2F)S(=O)(=O)N1] |
| 138 | 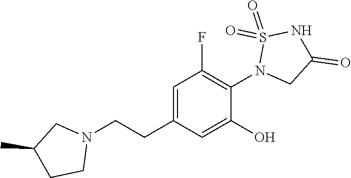 [SMILES: C[C@@H]1CCN(CCc2cc(O)c(N3CC(=O)NS3(=O)=O)c(F)c2)C1] |
| 139 | 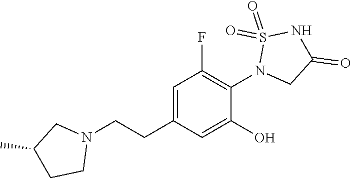 [SMILES: C[C@H]1CCN(CCc2cc(O)c(N3CC(=O)NS3(=O)=O)c(F)c2)C1] |
| 140 | 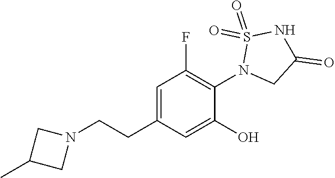 [SMILES: CC1CN(CCc2cc(O)c(N3CC(=O)NS3(=O)=O)c(F)c2)C1] |
| 141 | 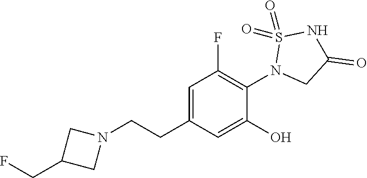 [SMILES: O=C1CN(c2c(O)cc(CCN3CC(CF)C3)cc2F)S(=O)(=O)N1] |
| 142 | 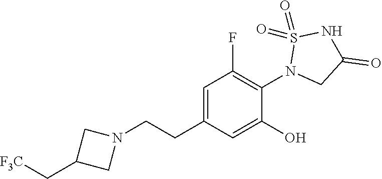 [SMILES: O=C1CN(c2c(O)cc(CCN3CC(CC(F)(F)F)C3)cc2F)S(=O)(=O)N1] |

*Stereochemistry arbitrarily assigned

In some embodiments, the compound disclosed herein is a compound selected from Table 2, or a pharmaceutically acceptable salt, solvate, or stereoisomer thereof.

TABLE 2
Structure

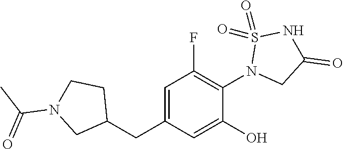 [SMILES: CC(=O)N1CCC(Cc2cc(O)c(N3CC(=O)NS3(=O)=O)c(F)c2)C1]

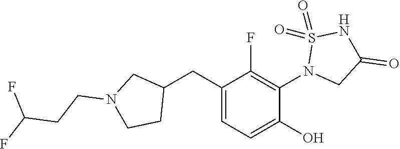 [SMILES: O=C1CN(c2c(O)ccc(CC3CCN(CCC(F)F)C3)c2F)S(=O)(=O)N1]

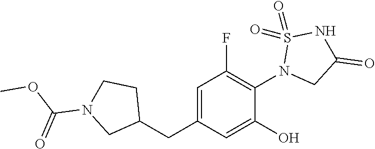 [SMILES: COC(=O)N1CCC(Cc2cc(O)c(N3CC(=O)NS3(=O)=O)c(F)c2)C1]

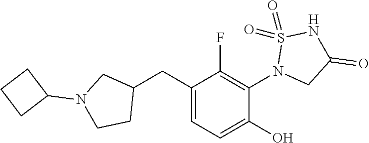 [SMILES: O=C1CN(c2c(O)ccc(CC3CCN(C4CCC4)C3)c2F)S(=O)(=O)N1]

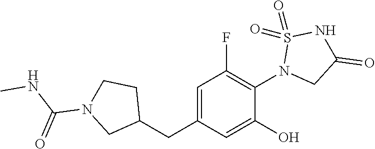 [SMILES: CNC(=O)N1CCC(Cc2cc(O)c(N3CC(=O)NS3(=O)=O)c(F)c2)C1]

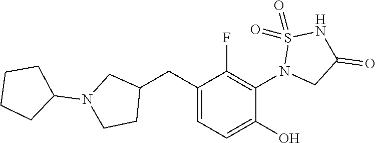 [SMILES: O=C1CN(c2c(O)ccc(CC3CCN(C4CCCC4)C3)c2F)S(=O)(=O)N1]

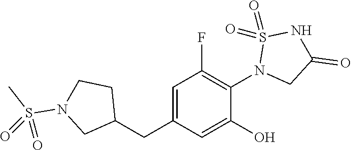 [SMILES: CS(=O)(=O)N1CCC(Cc2cc(O)c(N3CC(=O)NS3(=O)=O)c(F)c2)C1]

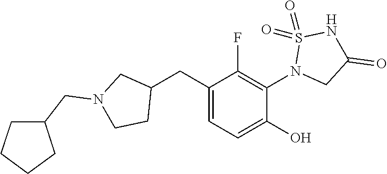 [SMILES: O=C1CN(c2c(O)ccc(CC3CCN(CC4CCCC4)C3)c2F)S(=O)(=O)N1]

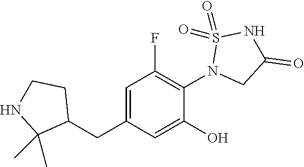 [SMILES: CC1(C)NCCC1Cc1cc(O)c(N2CC(=O)NS2(=O)=O)c(F)c1]

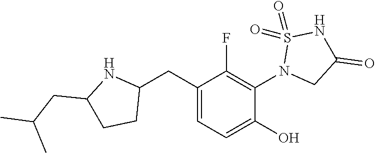 [SMILES: CC(C)CC1CCC(Cc2ccc(O)c(N3CC(=O)NS3(=O)=O)c2F)N1]

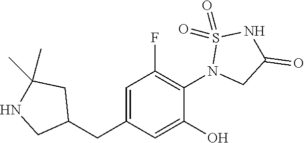 [SMILES: CC1(C)CC(Cc2cc(O)c(N3CC(=O)NS3(=O)=O)c(F)c2)CN1]

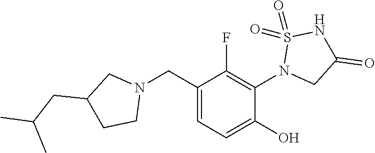 [SMILES: CC(C)CC1CCN(Cc2ccc(O)c(N3CC(=O)NS3(=O)=O)c2F)C1]

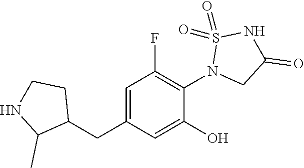 [SMILES: CC1NCCC1Cc1cc(O)c(N2CC(=O)NS2(=O)=O)c(F)c1]

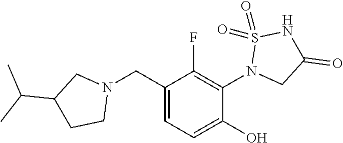 [SMILES: CC(C)C1CCN(Cc2ccc(O)c(N3CC(=O)NS3(=O)=O)c2F)C1]

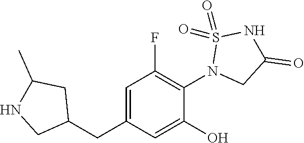 [SMILES: CC1CC(Cc2cc(O)c(N3CC(=O)NS3(=O)=O)c(F)c2)CN1]

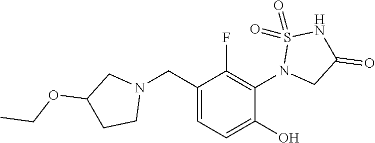 [SMILES: CCOC1CCN(Cc2ccc(O)c(N3CC(=O)NS3(=O)=O)c2F)C1]

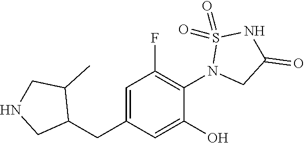 [SMILES: CC1CNCC1Cc1cc(O)c(N2CC(=O)NS2(=O)=O)c(F)c1]

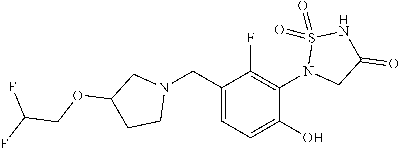 [SMILES: O=C1CN(c2c(O)ccc(CN3CCC(OCC(F)F)C3)c2F)S(=O)(=O)N1]

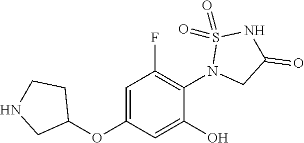 [SMILES: O=C1CN(c2c(O)cc(OC3CCNC3)cc2F)S(=O)(=O)N1]

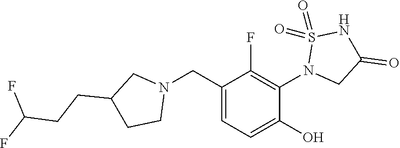 [SMILES: O=C1CN(c2c(O)ccc(CN3CCC(CCC(F)F)C3)c2F)S(=O)(=O)N1]

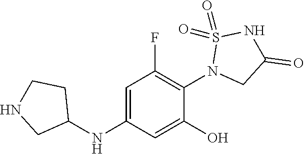 [SMILES: O=C1CN(c2c(O)cc(NC3CCNC3)cc2F)S(=O)(=O)N1]

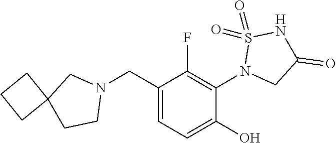 [SMILES: O=C1CN(c2c(O)ccc(CN3CCC4(CCC4)C3)c2F)S(=O)(=O)N1]

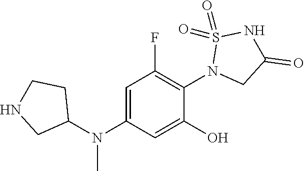 [SMILES: CN(c1cc(O)c(N2CC(=O)NS2(=O)=O)c(F)c1)C1CCNC1]

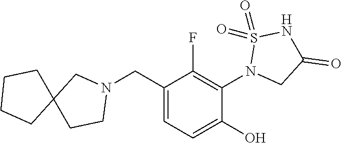 [SMILES: O=C1CN(c2c(O)ccc(CN3CCC4(CCCC4)C3)c2F)S(=O)(=O)N1]

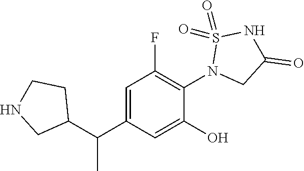 [SMILES: CC(c1cc(O)c(N2CC(=O)NS2(=O)=O)c(F)c1)C1CCNC1]

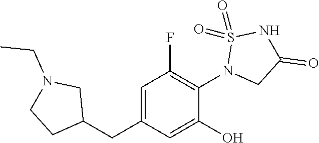 [SMILES: CCN1CCC(Cc2cc(O)c(N3CC(=O)NS3(=O)=O)c(F)c2)C1]

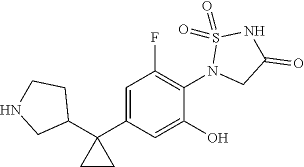 [SMILES: O=C1CN(c2c(O)cc(C3(C4CCNC4)CC3)cc2F)S(=O)(=O)N1]

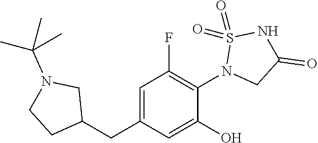 [SMILES: CC(C)(C)N1CCC(Cc2cc(O)c(N3CC(=O)NS3(=O)=O)c(F)c2)C1]

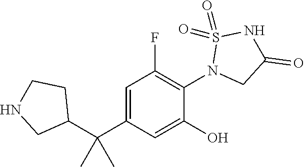 [SMILES: CC(C)(c1cc(O)c(N2CC(=O)NS2(=O)=O)c(F)c1)C1CCNC1]

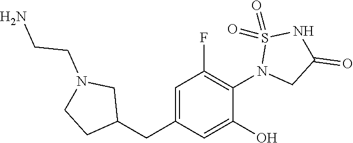 [SMILES: NCCN1CCC(Cc2cc(O)c(N3CC(=O)NS3(=O)=O)c(F)c2)C1]

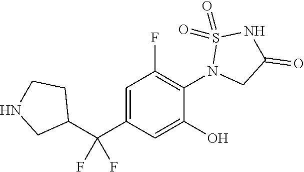 [SMILES: O=C1CN(c2c(O)cc(C(F)(F)C3CCNC3)cc2F)S(=O)(=O)N1]

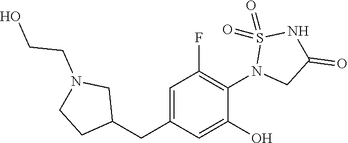 [SMILES: O=C1CN(c2c(O)cc(CC3CCN(CCO)C3)cc2F)S(=O)(=O)N1]

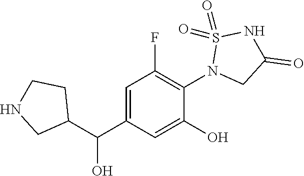 [SMILES: O=C1CN(c2c(O)cc(C(O)C3CCNC3)cc2F)S(=O)(=O)N1]

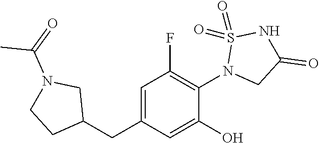 [SMILES: CC(=O)N1CCC(Cc2cc(O)c(N3CC(=O)NS3(=O)=O)c(F)c2)C1]

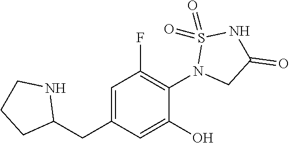 [SMILES: O=C1CN(c2c(O)cc(CC3CCCN3)cc2F)S(=O)(=O)N1]

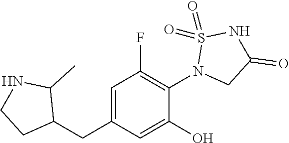 [SMILES: CC1NCCC1Cc1cc(O)c(N2CC(=O)NS2(=O)=O)c(F)c1]

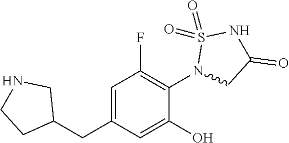 [SMILES: O=C1CN(c2c(O)cc(CC3CCNC3)cc2F)S(=O)(=O)N1]

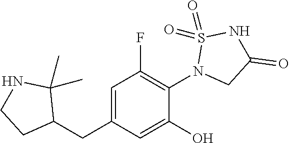 [SMILES: CC1(C)NCCC1Cc1cc(O)c(N2CC(=O)NS2(=O)=O)c(F)c1]

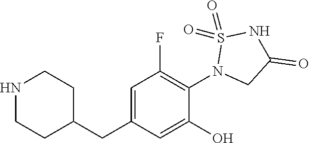 [SMILES: O=C1CN(c2c(O)cc(CC3CCNCC3)cc2F)S(=O)(=O)N1]

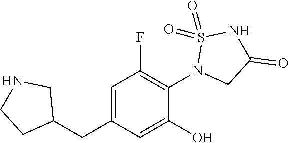 [SMILES: O=C1CN(c2c(O)cc(CC3CCNC3)cc2F)S(=O)(=O)N1]

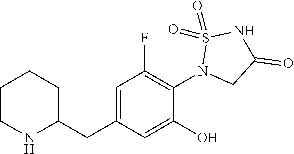 [SMILES: O=C1CN(c2c(O)cc(CC3CCCCN3)cc2F)S(=O)(=O)N1]

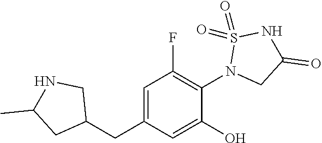 [SMILES: CC1CC(Cc2cc(O)c(N3CC(=O)NS3(=O)=O)c(F)c2)CN1]

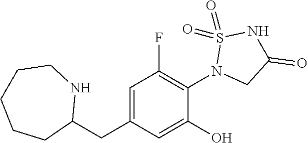 [SMILES: O=C1CN(c2c(O)cc(CC3CCCCCN3)cc2F)S(=O)(=O)N1]

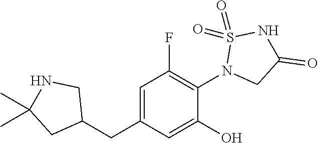 [SMILES: CC1(C)CC(Cc2cc(O)c(N3CC(=O)NS3(=O)=O)c(F)c2)CN1]

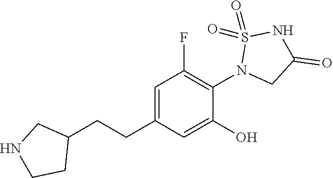 [SMILES: O=C1CN(c2c(O)cc(CCC3CCNC3)cc2F)S(=O)(=O)N1]

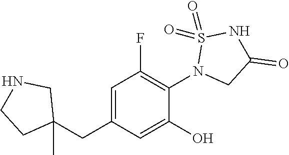 [SMILES: CC1(Cc2cc(O)c(N3CC(=O)NS3(=O)=O)c(F)c2)CCNC1]

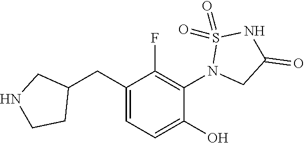 [SMILES: O=C1CN(c2c(O)ccc(CC3CCNC3)c2F)S(=O)(=O)N1]

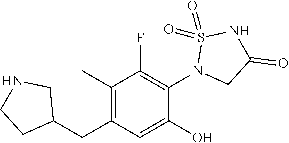 [SMILES: Cc1c(CC2CCNC2)cc(O)c(N2CC(=O)NS2(=O)=O)c1F]

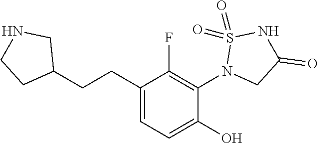 [SMILES: O=C1CN(c2c(O)ccc(CCC3CCNC3)c2F)S(=O)(=O)N1]

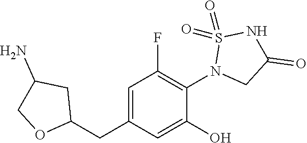 [SMILES: NC1COC(Cc2cc(O)c(N3CC(=O)NS3(=O)=O)c(F)c2)C1]

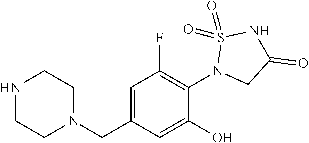 [SMILES: O=C1CN(c2c(O)cc(CN3CCNCC3)cc2F)S(=O)(=O)N1]

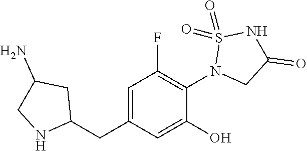 [SMILES: NC1CNC(Cc2cc(O)c(N3CC(=O)NS3(=O)=O)c(F)c2)C1]

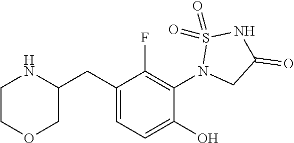 [SMILES: O=C1CN(c2c(O)ccc(CC3COCCN3)c2F)S(=O)(=O)N1]

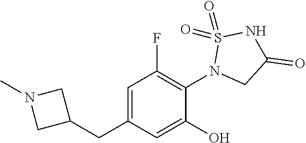 [SMILES: CN1CC(Cc2cc(O)c(N3CC(=O)NS3(=O)=O)c(F)c2)C1]

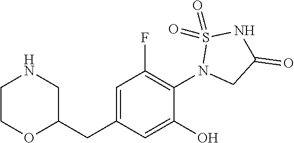 [SMILES: O=C1CN(c2c(O)cc(CC3CNCCO3)cc2F)S(=O)(=O)N1]

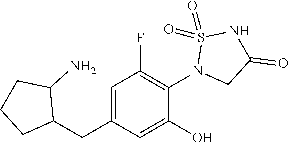 [SMILES: NC1CCCC1Cc1cc(O)c(N2CC(=O)NS2(=O)=O)c(F)c1]

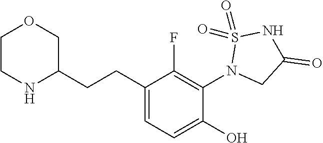 [SMILES: O=C1CN(c2c(O)ccc(CCC3COCCN3)c2F)S(=O)(=O)N1]

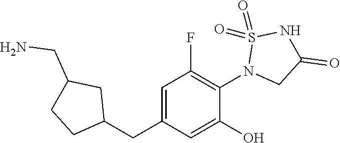 [SMILES: NCC1CCC(Cc2cc(O)c(N3CC(=O)NS3(=O)=O)c(F)c2)C1]

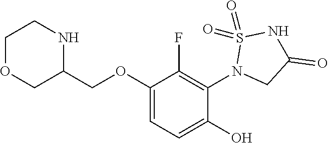 [SMILES: O=C1CN(c2c(O)ccc(OCC3COCCN3)c2F)S(=O)(=O)N1]

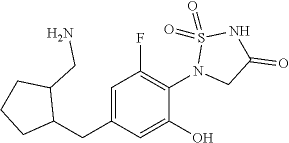 [SMILES: NCC1CCCC1Cc1cc(O)c(N2CC(=O)NS2(=O)=O)c(F)c1]

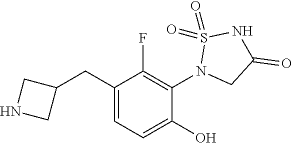 [SMILES: O=C1CN(c2c(O)ccc(CC3CNC3)c2F)S(=O)(=O)N1]

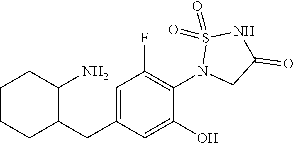 [SMILES: NC1CCCCC1Cc1cc(O)c(N2CC(=O)NS2(=O)=O)c(F)c1]

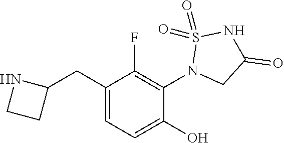 [SMILES: O=C1CN(c2c(O)ccc(CC3CCN3)c2F)S(=O)(=O)N1]

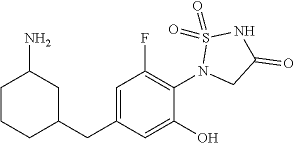 [SMILES: NC1CCCC(Cc2cc(O)c(N3CC(=O)NS3(=O)=O)c(F)c2)C1]

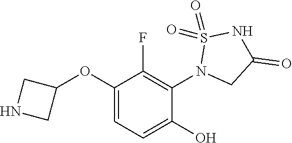 [SMILES: O=C1CN(c2c(O)ccc(OC3CNC3)c2F)S(=O)(=O)N1]

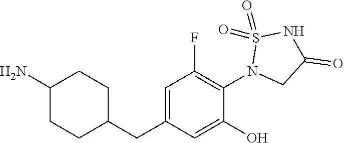 [SMILES: NC1CCC(Cc2cc(O)c(N3CC(=O)NS3(=O)=O)c(F)c2)CC1]

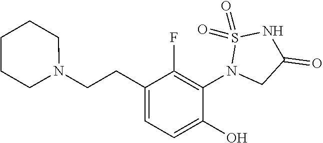 [SMILES: O=C1CN(c2c(O)ccc(CCN3CCCCC3)c2F)S(=O)(=O)N1]

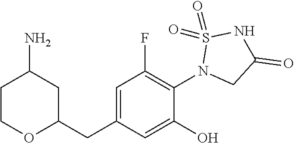 [SMILES: NC1CCOC(Cc2cc(O)c(N3CC(=O)NS3(=O)=O)c(F)c2)C1]

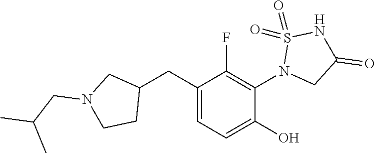 [SMILES: CC(C)CN1CCC(Cc2ccc(O)c(N3CC(=O)NS3(=O)=O)c2F)C1]

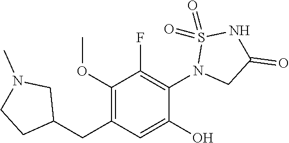 [SMILES: COc1c(CC2CCN(C)C2)cc(O)c(N2CC(=O)NS2(=O)=O)c1F]

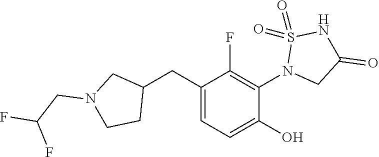 [SMILES: O=C1CN(c2c(O)ccc(CC3CCN(CC(F)F)C3)c2F)S(=O)(=O)N1]

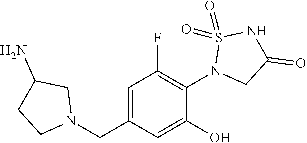 [SMILES: NC1CCN(Cc2cc(O)c(N3CC(=O)NS3(=O)=O)c(F)c2)C1]

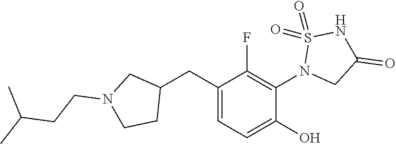 [SMILES: CC(C)CCN1CCC(Cc2ccc(O)c(N3CC(=O)NS3(=O)=O)c2F)C1]

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺ (C₁₋₄ alkyl) 4, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Method of Treatment

Disclosed herein are methods of treatment of a disease in which inhibition of PTPN1/PTPN2 is beneficial, the method comprising administering a compound disclosed herein, or a pharmaceutically acceptable salt, solvate, or stereoisomer thereof.

Disclosed herein are methods of treatment of a disease in which inhibition of PTPN1 is beneficial, the method comprising administering a compound disclosed herein, or a pharmaceutically acceptable salt, solvate, or stereoisomer thereof. In some embodiments, the disease in which inhibition of PTPN1 is beneficial is cancer or a metabolic disease.

Disclosed herein are methods of treatment of a disease in which inhibition of PTPN2 is beneficial, the method comprising administering a compound disclosed herein, or a pharmaceutically acceptable salt, solvate, or stereoisomer thereof. In some embodiments, the disease in which inhibition of PTPN2 is beneficial is cancer.

### Cancer

In some embodiments, the compound disclosed herein, or a pharmaceutically acceptable salt, solvate, or stereoisomer thereof, is used to treat cancer.

As used herein, “cancer” refers to human cancers and carcinomas, sarcomas, adenocarcinomas (e.g., papillary adenocarcinomas), lymphomas, leukemias, melanomas, etc., including solid and lymphoid cancers.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, chronic leukemia, acute nonlymphocytic leukemia, acute lymphocytic leukemia, B-cell chronic lymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, acute myelocytic leukemia, chronic myelocytic leukemia, leukemia cutis, embryonal leukemia, eosinophilic leukemia, erythroleukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocyte leukemia, micromyeloblastic leukemia, monocytic leukemia, myeloblasts leukemia, myelocytic leukemia, myeloid granulocytic leukemia, myelomonocytic leukemia, Naegeli leukemia, plasma cell leukemia, multiple myeloma, plasmacytic leukemia, polycythemia vera, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, leiomyosarcoma, lymphosarcoma, lymphangiosarcoma, lymphangioendotheliosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, endotheliosarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, osteogenic sarcoma, parosteal sarcoma, reticulocytic sarcoma, Rous sarcoma, serocystic sarcoma, synovial sarcoma, or telangiectaltic sarcoma.

The term “carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bile duct carcinoma, bladder carcinoma, breast carcinoma, Brenner carcinoma, bronchioalveolar carcinoma, bronchiolar carcinoma, bronchiogenic carcinoma, cerebriform carcinoma, cervical carcinoma, cholangiocellular carcinoma, chordoma, chorionic carcinoma, clear cell carcinoma, colloid carcinoma, colon carcinoma, comedo carcinoma, corpus carcinoma, cribriform carcinoma, carcinoma en cuirasse, carcinoma cutaneum, cylindrical carcinoma, cylindrical cell carcinoma, cystadenocarcinoma, duct carcinoma, ductal carcinoma, carcinoma durum, embryonal carcinoma, encephaloid carcinoma, endometrioid carcinoma, epiermoid carcinoma, epithelial carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lung carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, nonpapillary renal cell carcinoma, oat cell carcinoma, carcinoma ossificans, osteoid carcinoma, ovarian carcinoma, pancreatic ductal carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, sebaceous gland carcinoma, seminoma, serous carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, sweat gland carcinoma, carcinoma telangiectaticum, carcinoma telangiectodes, transitional cell carcinoma, carcinoma tuberosum, tubular carcinoma, tuberous carcinoma, undifferentiated carcinoma, verrucous carcinoma, or carcinoma villosum.

In some embodiments, the cancer is acoustic neuroma, adrenal cortical cancer, adrenal gland cancer, astrocytoma, benign monoclonal gammopathy, biliary tract cancer, bladder cancer, bone cancer, brain tumor, breast cancer, bronchus cancer, cancer of the hematological tissues, cancer of the hepatic stellate cells, cancer of the oral cavity or pharynx, cancer of the pancreatic stellate cells, carcinoma, central nervous system cancer, cervical cancer, colon cancer, colorectal cancer, craniopharyngioma, ductal carcinoma, endocrine system cancer, endometrial cancer, ependymoma, epithelial ovarian cancer, esophageal cancer, gastric cancer, genitourinary tract cancer, glioblastoma multiforme, glioma, gynecologic cancers, head and neck cancer, hemangioblastoma, Hodgkin's Disease, immunocytic amyloidosis, kidney cancer, laryngeal cancer, leukemia, liver cancer (including hepatocarcinoma), lobular carcinoma, lung cancer, lymphoma, malignant carcinoid, malignant hypercalcemia, malignant pancreatic insulanoma, medullary thyroid cancer, medulloblastoma, melanoma, meningioma, mesothelioma, multiple myeloma, myeloma, neoplasms of the endocrine or exocrine pancreas, neuroblastoma, non-Hodgkin's Lymphoma, oligodendroglioma, oral cancer, ovarian cancer, Paget's Disease of the Nipple, pancreatic cancer, papillary thyroid cancer, peripheral nervous system cancer, Phyllodes Tumors, pinealoma, premalignant skin lesions, primary macroglobulinemia, primary thrombocytosis, prostate cancer, renal cancer, retinoblastoma, rhabdomyosarcoma, salivary gland cancer, sarcoma, skin cancer, small bowel or appendix cancer, stomach cancer, testicular cancer, thyroid cancer, urinary bladder cancer, uterine cancer, Waldenstrom's macroglobulinemia.

### Metabolic Diseases

In some embodiments, the compound disclosed herein, or a pharmaceutically acceptable salt, solvate, or stereoisomer thereof, is used to treat a metabolic disease.

As used herein, the term “metabolic disease” refers to a disease or condition affecting a metabolic process in a subject. Exemplary metabolic diseases include non-alcoholic steatohepatitis (NASH), non-alcoholic fatty liver disease (NAFLD), liver fibrosis, obesity, heart disease, atherosclerosis, arthritis, cystinosis, diabetes (e.g., Type I diabetes, Type II diabetes, or gestational diabetes), metabolic syndrome, phenylketonuria, proliferative retinopathy, or Kearns-Sayre disease. In some embodiments, a compound disclosed herein, is used to treat a metabolic disease (e.g., a metabolic disease described herein) by decreasing or eliminating a symptom of the disease. In some embodiments, the method of treatment comprises decreasing or eliminating a symptom comprising elevated blood pressure, elevated blood sugar level, weight gain, fatigue, blurred vision, abdominal pain, flatulence, constipation, diarrhea, jaundice, and the like.

### Dosing

In certain embodiments, the compositions containing the compound(s) described herein are administered for therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”).

Once improvement of the patient's conditions has occurred, a maintenance dose is administered if necessary. Subsequently, in specific embodiments, the dosage, or the frequency of administration, or both, is reduced, as a function of the symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In some embodiments, doses employed for adult human treatment are typically in the range of 0.01 mg-5000 mg per day. In some embodiments, the daily dosages appropriate for the compound described herein, or a pharmaceutically acceptable salt thereof, are from about 0.01 to about 50 mg/kg per body weight. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In some embodiments, the compounds described herein are administered to animals.

In another aspect, provided herein are pharmaceutical compositions comprising a compound described herein, or a pharmaceutically acceptable salt, solvate, or stereoisomer thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

### Combination

Disclosed herein are methods of treating cancer using a compound disclosed herein, or a pharmaceutically acceptable salt, solvate, or stereoisomer thereof, in combination with an additional therapeutic agent.

In some embodiments, the additional therapeutic agent is an anticancer agent.

In some embodiments, the additional therapeutic agent is administered at the same time as the compound disclosed herein. In some embodiments, the additional therapeutic agent and the compound disclosed herein are administered sequentially. In some embodiments, the additional therapeutic agent is administered less frequently than the compound disclosed herein. In some embodiments, the additional therapeutic agent is administered more frequently than the compound disclosed herein. In some embodiments, the additional therapeutic agent is administered prior than the administration of the compound disclosed herein. In some embodiments, the additional therapeutic agent is administered after the administration of the compound disclosed herein.

In some embodiments, the additional therapeutic agent is an immunotherapeutic agent. In some embodiments, the immunotherapeutic agent is an anti-PD-1 antibody, an anti-PD-L1 antibody, or an anti-CTLA-4 antibody.

### Example 1: Synthesis of 5-(2-fluoro-6-hydroxy-4-((1-methylpyrrolidin-3-yl)methyl)phenyl)-1,2,5-thiadiazolidin-3-one 1,1-dioxide

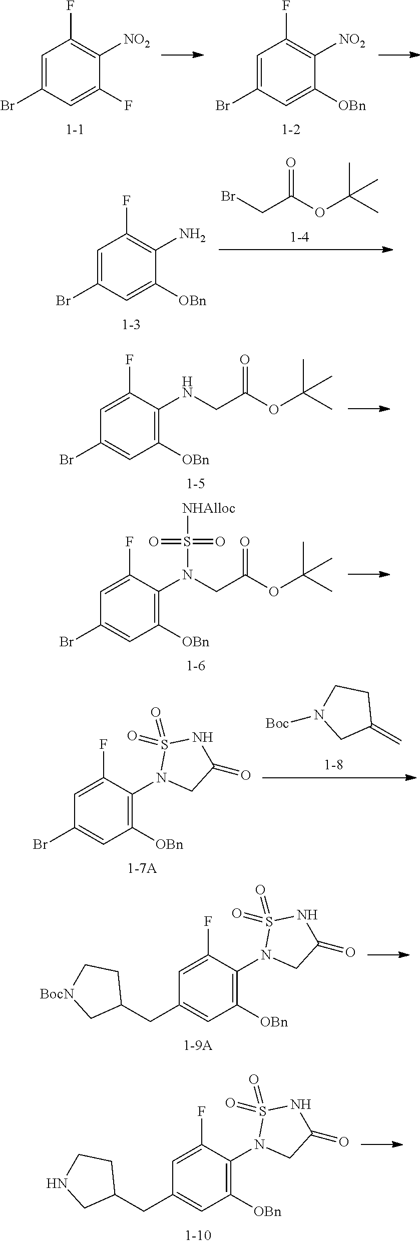

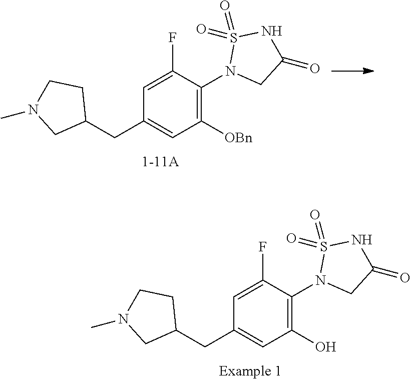 [SMILES: CN1CCC(Cc2cc(F)c(N3CC(=O)NS3(=O)=O)c(OCc3ccccc3)c2)C1 ; CN1CCC(Cc2cc(O)c(N3CC(=O)NS3(=O)=O)c(F)c2)C1]

### Step 1: Synthesis of Compound 1-2

To a solution of 1-1 (100 g, 420.1 mmol, 1 eq) in THE (1.5 L) was added benzyl alcohol (47.7 g, 441.2 mmol, 45.8 mL, 1.05 eq) followed by dropwise addition of 1M t-BuOK (441 mL, 1.05 eq) at −78° C. and then allowed to stir for 0.5 h. To the resulting reaction mixture was then added to H₂O (1.5 L) then extracted with ethyl acetate (1 L×3). The combined organic layers were washed with brine (2 L), dried with Na₂SO₄, then filtered and concentrated under reduced pressure to give a crude. The crude was recrystallized in heptane to give 1-2 (131 g, 1.21 mol, 95% yield) as a yellow solid. ¹H NMR (400 MHz, DMSO-d₆) δ 7.62 (d, J=1.6 Hz, 1H), 7.56 (dd, J=9.2, 1.6 Hz, 1H), 7.41 (m, 5H), 5.35 (s, 2H).

### Step 2: Synthesis of Compound 1-3

To a solution of 1-2 (90 g, 275.97 mmol, 1 eq) in THE (500 mL) and MeOH (500 mL) was added Zn (90.2 g, 1.38 mol, 5 eq) portion-wise, followed by slow addition of sat. NH₄Cl_{(aq)} (54.67 g, 275.97 mmol, 100 mL, 27% purity, 1 eq) then allowed to stir for 3 h at 25° C. The reaction mixture was filtered, then poured into water (1 L) and extracted with ethyl acetate (500 mL×4). The combined organic layers were washed with brine (1 L), dried with Na₂SO₄, then filtered and concentrated under reduced pressure to give 1-3 (78 g, 95% yield). ¹H NMR (400 MHz, DMSO-d₆) δ 7.48 (m, 2H), 7.39 (m, 2H), 7.31 (m, 1H), 5.16 (s, 2H), 4.84 (s, 2H).

### Step 3: Synthesis of Compound 1-5

To a solution 1-3 (103 g, 347.81 mmol, 1 eq) in DMF (1.5 L) was added K₂CO₃ (144 g, 1.04 mol, 3 eq), H₂O (6.2 mL) and 1-4 (74.6 g, 382 mmol, 56.5 mL, 1.1 eq) then heated to 60° C. and allowed to stir for 72 h. To the resulting reaction mixture was added H₂O (1 L) then extracted with ethyl acetate (500 mL×4). The combined organic layers were washed with brine (500 mL), dried with Na₂SO₄, then filtered and concentrated under reduced pressure to give a crude. The crude was then purified by column chromatography on silica gel eluting with petroleum ether/ethyl acetate-1:0 to 10:1 to give crude 1-5 (160 g, crude product) as a brown oil. LCMS (ESI+): m/z=356.0/354.1 (M−tBu+H)⁺.

### Step 4: Synthesis of Compound 1-6

To a solution of N-(oxomethylene)sulfamoyl chloride (43.1 g, 304 mmol, 26.4 mL, 2.5 eq) in CH₂Cl₂ (210 mL) was added prop-2-en-1-ol (17.7 g, 304 mmol, 20.7 mL, 2.5 eq) and allowed to stir for 0.5 h at 0° C. To this was then added a solution of 1-5 (50 g, 121.87 mmol, 1 eq) and triethylamine (37 g, 365.61 mmol, 50.89 mL, 3 eq) in CH₂Cl₂ (280 mL) and allowed to stir for an additional 0.5 h. The reaction mixture was then allowed to warm to 25° C. and stirred for an additional 1 h. The resulting reaction mixture was added to H₂O (600 mL) then extracted with CH₂Cl₂ (600 mL×3). The combined organic layer was washed with brine (600 mL), dried with Na₂SO₄, then filtered and concentrated under reduced pressure to give 1-6 (510 g, crude) as a yellow solid which was used in next step without purification. ¹H NMR (400 MHz, DMSO-d₆) δ 11.68 (s, 1H), 7.47-7.39 (m, 4H), 7.38-7.34 (m, 1H), 7.30-7.25 (m, 2H), 5.76-5.73 (m, 1H), 5.26-5.23 (m, 1H), 5.19 (d, J=1.7 Hz, 1H), 5.18-5.13 (m, 2H), 4.59 (d, J=17.5 Hz, 1H), 4.31-4.12 (m, 3H), 1.34 (s, 9H).

### Step 5: Synthesis of Compound 1-7A

To a solution of 1-6 (50 g, 87 mmol, 1 eq) in MeOH (500 mL) was added NaOMe (78.5 g, 435 mmol, 30% purity in THF, 5 eq) and Pd(PPh₃)₄ (3.02 g, 2.62 mmol, 0.03 eq) then heated to 60° C. and allowed to stir for 1 h. The resulting reaction mixture was filtered, and the filtrate was isolated. To the filtrate was then added to 1M HCl (2 L) then extracted with ethyl acetate (3 L×3). The combined organic layer was washed with brine (1 L), dried with Na₂SO₄, then filtered and concentrated under reduced pressure to give a crude. The crude was then purified by HPLC to give 1-7A (196 g, 472 mmol, 54% yield) as a yellow solid. ¹H NMR (400 MHz, CD₃OD) δ 7.49-7.44 (m, 2H), 7.40-7.33 (m, 3H), 7.22 (t, J=1.8 Hz, 1H), 7.12 (dd, J=2.0, 8.8 Hz, 1H), 5.17 (s, 2H), 4.40 (s, 2H).

### Step 6: Synthesis of Compound 1-9A

To a solution of 0.5M 9-BBN (180 mL, 1.5 eq) was added 1-8 (16.5 g, 90.3 mmol, 1.5 eq) then heated to 60° C. and allowed to stir 1 h. The reaction mixture was then allowed to cool to 20° C. To this was then added a solution of 1-7 (25 g, 60.2 mmol, 1 eq), K₂CO₃ (12.4 g, 90.3 mmol, 1.5 eq) and Pd(dppf)Cl₂—CH₂Cl₂ (4.92 g, 6.02 mmol, 0.1 eq) in DMF (250 mL) and H₂O (25 mL) then heated to 60° C. and allowed to stir for an additional 12 h. The resulting reaction mixture was filtered, and the filtrate was isolated. To the filtrate was then added to H₂O (200 mL) then extracted with ethyl acetate (100 mL×3). The combined organic layers were washed with brine (100 mL), dried with Na₂SO₄, then filtered and concentrated under reduced pressure to give a crude. The crude was then purified by HPLC to give 1-9A (22 g, 42.3 mmol, 70.33% yield) as a brown solid. ¹H NMR (400 MHz, CD₃OD) δ 7.51 (d, J 7.4 Hz, 2H), 7.41-7.22 (m, 3H), 6.78 (s, 1H), 6.69 (br d, J=10.3 Hz, 1H), 5.19 (s, 2H), 4.27 (s, 2H), 3.47-3.36 (m, 2H), 3.27-3.15 (m, 1H), 2.95 (br t, J=8.5 Hz, 1H), 2.75-2.56 (m, 2H), 2.49-2.33 (m, 1H), 1.86 (br d, J=5.9 Hz, 1H), 1.60-1.50 (m, 1H), 1.45 (s, 9H).

### Step 7: Synthesis of Compound 1-10

A solution of 1-9A (13 g, 25 mmol, 1 eq) in 4M HCl/ethyl acetate (200 mL) was allowed to stir for 2 h at 25° C. The reaction mixture was then concentrated under reduced pressure to give 1-10 (15.7 g, HCl salt, crude) as a brown solid. ¹H NMR (400 MHz, CD₃OD) δ 7.48 (br d, J=7.0 Hz, 2H), 7.40-7.30 (m, 3H), 6.90 (s, 1H), 6.79 (dd, J=0.8, 10.1 Hz, 1H), 5.21 (s, 2H), 4.42 (s, 2H), 3.45-3.34 (m, 1H), 3.23 (br s, 1H), 2.85 (br s, 1H), 2.78 (br dd, J=3.7, 7.4 Hz, 2H), 2.65-2.53 (m, 1H), 2.13-2.00 (m, 2H), 1.75-1.56 (m, 1H).

### Step 8: Synthesis of Compound 1-11A

To a solution of 1-10 (250 mg, 548 μmol, 1 eq) in MeOH (3 mL) was added formaldehyde (82 mg, 1.1 mmol, 75 μL, 2 eq) and AcOH (82 mg, 1.3 mmol, 78 μL, 2.5 eq) and allowed to stir for 0.5 h at 0° C. To this was then added NaBH₄ (130 mg, 3.4 mmol, 6.27 eq) and allowed to stir for an additional 2 h at 0° C. The resulting reaction mixture was added to H₂O (50 mL) then extracted with ethyl acetate (5 mL×3). The combined organic layer was washed with brine, dried with Na₂SO₄, then filtered and concentrated under reduced pressure to give 1-11A as a crude. LCMS (ESI−): m/z=432.4 (M−H)⁻.

### Step 9: Synthesis of Compound Example 1

To a solution of 1-11A (150 mg, 319 μmol, 1 eq) in MeOH (5 mL) was added Pd/C (100 mg, 5% purity) then degassed and purged with H₂ (15 psi) several times. The reaction mixture was then allowed to stir for 12 h at 15° C. The resulting reaction mixture was filtered then concentrated under reduced pressure to give a crude. The crude was purified by HPLC to give Example 1 (6 mg, 98% purity). LCMS (ESI−): m/z 342.2 (M−H)⁻. ¹H NMR (400 MHz, CD₃OD) δ 6.61 (m, 2H), 4.23 (s, 2H), 2.81 (m, 4H), 2.69 (m, 3H), 2.21 (m, 1H), 1.78 (m, 1H), 1.32 (m, 1H).

Examples 2-30 were prepared according to the procedures described in Example 1 using the appropriate intermediates.

| Example | LCMS | ¹H NMR |
|---|---|---|
| 2 | LCMS (ESI−): | ¹H NMR (400 MHz, CD₃OD) δ 6.65-6.53 (m, 2H), 4.24 (s, 2H), 3.54- |
| | m/z = 370.1 | 3.36 (m, 4H), 2.99 (br s, 1H), 2.81-2.60 (m, 3H), 2.29-2.05 (m, 1H), |
| | (M − H)⁻ | 1.76 (br dd, J = 8.9, 12.6 Hz, 1H), 1.35 (dd, J = 2.1, 6.5 Hz, 6H) |
| 3 | LCMS (ESI−): | ¹H NMR (400 MHz, D₂O) δ 6.68-6.59 (m, 2H), 4.30 (s, 2H), 3.20-3.06 |
| | m/z = 399.1 | (m, 5H), 2.96-2.86 (m, 2H), 2.72-2.49 (m, 10H), 2.04 (qd, J = 6.7, 13.1 |
| | (M − H)⁻ | Hz, 1H), 1.71-1.58 (m, 1H) |
| 4 | LCMS (ESI−): | ¹H NMR (400 MHz, D₂O) δ 8.19 (s, 1H), 7.27 (s, 1H), 6.69-6.51 (m, |
| | m/z = 409.1 | 2H), 4.31 (br d, J = 19.5 Hz, 4H), 3.35-3.17 (m, 3H), 2.92-2.80 (m, 1H), |
| | (M − H)⁻ | 2.62 (br s, 3H), 2.06 (br dd, J = 5.9, 12.6 Hz, 1H), 1.67 (br dd, J = 7.6, |
| | | 13.0 Hz, 1H). |
| 5 | LCMS (ESI−): | ¹H NMR (400 MHz, CD₃OD) δ 6.66-6.53 (m, 2H), 4.23 (s, 2H), 3.87- |
| | m/z = 439.2 | 3.35 (m, 4H), 3.28-3.01 (m, 4H), 2.94 (br t, J = 5.9 Hz, 1H), 2.87 (br s, |
| | (M − H)⁻ | 2H), 2.78-2.68 (m, 2H), 2.68-2.58 (m, 1H), 2.23-2.04 (m, 2H), 1.96- |
| | | 1.83 (m, 1H), 1.78-1.66 (m, 3H), 1.63-1.53 (m, 1H), 1.37 (br t, J = 9.6 |
| | | Hz, 2H) |
| 6 | LCMS (ESI−): | ¹H NMR (400 MHz, CD₃OD) δ 8.01 (s, 1H), 7.26 (s, 1H), 6.65-6.51 (m, |
| | m/z = 409.1 | 2H), 4.53 (s, 2H), 4.24 (s, 2H), 3.52-3.39 (m, 3H), 3.12-2.99 (m, 1H), |
| | (M − H)⁻ | 2.75-2.65 (m, 3H), 2.24-2.09 (m, 1H), 1.85-1.71 (m, 1H) |
| 7 | LCMS (ESI−): | ¹H NMR (400 MHz, CD₃OD) δ 7.65 (d, J = 1.6 Hz, 1H), 6.67 (d, J = 3.3 |
| | m/z = 408.1 | Hz, 1H), 6.61-6.50 (m, 3H), 4.43 (s, 2H), 4.23 (s, 2H), 3.51-3.34 (m, |
| | (M − H)⁻ | 3H), 3.03 (br d, J = 8.9 Hz, 1H), 2.70 (s, 3H), 2.24-2.10 (m, 1H), 1.86- |
| | | 1.70 (m, 1H) |
| 8 | LCMS (ESI−): | ¹H NMR (400 MHz, D₂O) δ 6.58 (m, 2H), 4.29 (s, 2H), 3.41 (m, 3H), |
| | m/z = 384.1 | 2.95 (m, 2H), 2.63 (m, 3H), 2.13 (m, 1H), 1.95 (m, 1H), 1.24 (m, 1H), |
| | (M − H)⁻ | 0.85 (t, 3H) |
| 9 | LCMS (ESI+): | ¹H NMR (400 MHz, D₂O) δ 6.13 (m, 2H), 4.28 (s, 2H), 3.41 (m, 3H), |
| | m/z = 386.1 | 3.37 (m, 2H), 2.69 (m, 3H), 2.03 (m, 1H), 1.71 (m, 4H), 1.31 (m, 2H), |
| | (M + H)⁺ | 0.81 (d, 6H) |
| 10 | LCMS (ESI−): | ¹H NMR (400 MHz, D₂O) δ 6.13 (m, 2H), 4.38 (s, 2H), 3.62 (m, 3H), |
| | m/z = 382.1 | 3.37 (m, 2H), 2.69 (m, 3H), 2.03 (m, 1H), 1.71 (m, 4H), 1.31 (m, 2H), |
| | (M − H)⁻ | 1.12 (m, 4H) |
| 11 | LCMS (ESI−): | ¹H NMR (400 MHz, D₂O) δ 6.68 (m, 2H), 3.66 (m, 1H), 3.09 (m, 4H), |
| | m/z = 370.1 | 2.72 (m, 3H), 2.14 (m, 1H), 1.71 (m, 4H), 0.92 (m, 3H) |
| | (M − H)⁻ | |
| 12 | LCMS (ESI−): | ¹H NMR (400 MHz, CD₃OD) 87.76 (s, 1H), 7.61 (s, 1H), 6.68-6.48 (m, |
| | m/z = 408.1 | 3H), 4.25 (d, J = 7.8 Hz, 4H), 3.44 (br d, J = 2.1 Hz, 3H), 3.02 (br d, J = |
| | (M − H)⁻ | 2.6 Hz, 1H), 2.70 (br s, 3H), 2.23-2.10 (m, 1H), 1.88-1.68 (m, 1H) |
| 13 | LCMS (ESI−): | ¹H NMR (400 MHz, D₂O) δ 6.63 (m, 2H), 4.25 (m, 2H), 3.39 (m, 1H), |
| | m/z = 412.1 | 3.35 (m, 2H), 3.21 (m, 2H), 2.95 (m, 1H), 2.75 (m, 3H), 2.23 (m, 1H), |
| | (M − H)⁻ | 1.75 (m, 3H), 1.38 (m, 6H), 0.95 (m, 3H) |
| 14 | LCMS (ESI+): | ¹H NMR (400 MHz, D₂O) δ 6.50-6.23 (m, 2H), 4.31 (s, 2H), 4.23-4.13 |
| | m/z = 414.1 | (m, 1H), 3.92-3.72 (m, 2H), 3.41-3.16 (m, 3H), 3.05 (br d, J = 6.8 Hz, |
| | (M + H) | 2H), 2.93-2.78 (m, 1H), 2.74-2.51 (m, 3H), 2.20-2.01 (m, 2H), 1.98- |
| | | 1.81 (m, 2H), 1.78-1.65 (m, 1H), 1.63-1.48 (m, 1H) |
| 15 | LCMS (ESI−): | ¹H NMR (400 MHz, CD₃OD) δ 6.66-6.54 (m, 2H), 4.23 (s, 2H), 3.95- |
| | m/z = 412.1 | 3.85 (m, 2H), 3.76 (q, J = 7.6 Hz, 1H), 3.60-3.35 (m, 5H), 3.24 (d, J = |
| | (M − H)⁻ | 7.3 Hz, 2H), 2.79-2.68 (m, 3H), 2.62 (td, J = 7.1, 14.4 Hz, 1H), 2.25- |
| | | 2.12 (m, 2H), 1.90-1.75 (m, 1H), 1.74-1.61 (m, 1H) |
| 16 | LCMS (ESI−): | ¹H NMR (400 MHz, CD₃OD) δ 8.26 (s, 1H), 8.12 (s, 1H), 6.64-6.53 (m, |
| | m/z = 409.1 | 2H), 4.35 (s, 2H), 4.23 (s, 2H), 3.57-3.40 (m, 3H), 3.06 (br d, J = 1.9 |
| | (M − H)⁻ | Hz, 1H), 2.77-2.65 (m, 3H), 2.18 (br dd, J = 5.7, 12.4 Hz, 1H), 1.79 (br |
| | | dd, J = 7.4, 12.6 Hz, 1H) |
| 17 | LCMS (ESI−): | ¹H NMR (400 MHz, CD₃OD) δ 7.25 (s, 2H), 6.66-6.50 (m, 2H), 4.29- |
| | m/z = 408.2 | 4.20 (m, 4H), 3.29-3.22 (m, 3H), 2.85 (br dd, J = 7.8, 10.5 Hz, 1H), |
| | (M − H)⁻ | 2.74-2.58 (m, 3H), 2.23-2.11 (m, 1H), 1.72 (br dd, J = 7.5, 13.1 Hz, 1H) |
| 18 | LCMS (ESI−): | ¹H NMR (400 MHz, CD₃OD) δ 7.72 (br s, 1H), 6.61-6.53 (m, 2H), 6.46 |
| | m/z = 408.1 | (d, J = 1.6 Hz, 1H), 4.36 (br s, 2H), 4.23 (s, 2H), 3.45 (br d, J = 14.8 Hz, |
| | (M − H)⁻ | 2H), 3.02 (br d, J = 3.1 Hz, 1H), 2.75-2.56 (m, 3H), 2.16 (br dd, J = 5.6, |
| | | 11.8 Hz, 1H), 1.85-1.69 (m, 1H), 1.44-1.19 (m, 1H) |
| 19 | LCMS (ESI−): | ¹H NMR (400 MHz, CD₃OD) δ 6.67-6.52 (m, 2H), 4.23-4.21 (m, 1H), |
| | m/z = 398.2 | 4.24 (s, 1H), 3.39 (br d, J = 8.6 Hz, 3H), 3.24-3.11 (m, 2H), 2.99 (br d, |
| | (M − H)⁻ | J = 3.8 Hz, 1H), 2.71 (br s, 3H), 2.16 (br s, 1H), 1.79 (br s, 1H), 1.71-1.53 |
| | | (m, 3H), 0.97 (d, J = 6.4 Hz, 6H) |
| 20 | LCMS (ESI+): | ¹H NMR (400 MHz, CD₃OD) δ 6.71-6.46 (m, 2H), 4.24 (s, 2H), 3.62- |
| | m/z = 400.1 | 3.36 (m, 3H), 3.14-2.95 (m, 3H), 2.76-2.61 (m, 3H), 2.21-2.02 (m, 1H), |
| | (M + H)⁺ | 1.77 (br d, J = 4.1 Hz, 1H), 1.08 (s, 9H) |
| 21 | LCMS (ESI+): | ¹H NMR (400 MHz, CD₃OD) δ 6.83-6.39 (m, 2H), 4.24 (s, 2H), 3.52- |
| | m/z = 412.1 | 3.33 (m, 3H), 3.13 (br d, J = 7.4 Hz, 2H), 2.96 (br d, J = 7.5 Hz, 1H), |
| | (M + H)⁺ | 2.70 (br s, 3H), 2.28-2.07 (m, 2H), 1.90 (dt, J = 6.8, 11.2 Hz, 2H), 1.83- |
| | | 1.74 (m, 1H), 1.81-1.57 (m, 4H), 1.34-1.20 (m, 2H) |
| 22 | LCMS (ESI+): | ¹H NMR (400 MHz, CD₃OD) δ 6.71-6.44 (m, 2H), 4.23 (s, 2H), 3.40- |
| | m/z = 398.1 | 3.33 (m, 1H), 3.30-3.24 (m, 2H), 3.15 (d, J = 7.3 Hz, 2H), 2.95-2.83 (m, |
| | (M + H)⁺ | 1H), 2.73-2.67 (m, 3H), 2.67-2.61 (m, 1H), 2.25-2.09 (m, 3H), 2.06- |
| | | 1.93 (m, 1H), 1.92-1.81 (m, 3H), 1.80 (br s, 1H) |
| 23 | LCMS (ESI+): | ¹H NMR (400 MHz, CD₃OD) δ6.71-6.47 (m, 2H), 4.23 (s, 2H), 3.55- |
| | m/z = 414.1 | 3.34 (m, 3H), 3.24-3.13 (m, 2H), 3.10-2.90 (m, 1H), 2.80-2.62 (m, 3H), |
| | (M + H)⁺ | 2.24-2.09 (m, 1H), 1.78 (br dd, J = 7.9, 12.5 Hz, 1H), 1.59 (td, J = 4.5, |
| | | 8.5 Hz, 2H), 0.97 (s, 9H) |
| 24 | LCMS (ESI+): | ¹H NMR (400 MHz, CD₃OD) δ6.70-6.48 (m, 2H), 4.23 (s, 2H), 3.51- |
| | m/z = 400.1 | 3.33 (m, 2H), 3.18-3.07 (m, 2H), 3.03-2.89 (m, 1H), 2.78-2.61 (m, 3H), |
| | (M + H)⁺ | 2.28-2.11 (m, 1H), 1.87-1.73 (m, 1H), 1.69 (td, J = 7.6, 15.4 Hz, 2H), |
| | | 1.46-1.30 (m, 4H), 0.95 (t, J = 6.9 Hz, 3H) |
| 25 | LCMS (ESI−): | ¹H NMR (400 MHz, CD₃OD) δ 6.59 (m, 2H), 3.93 (s, 2H), 3.20 (m, |
| | m/z = 328.1 | 3H), 2.76 (m, 1H), 2.58 (m, 2H), 2.48 (m, 1H), 1.98 (m, 1H), 1.53 (m, |
| | (M − H)⁻ | 1H) |
| 26 | LCMS (ESI−): | ¹H NMR (400 MHz, CD₃OD) δ 6.59 (m, 2H), 3.93 (s, 2H), 3.20 (m, |
| | m/z = 328.1 | 3H), 2.76 (m, 1H), 2.58 (m, 2H), 2.48 (m, 1H), 1.98 (m, 1H), 1.53 (m, |
| | (M − H)⁻ | 1H) |
| 27 | LCMS (ESI−): | ¹H NMR (400 MHz, CD₃OD) δ 6.59 (m, 2H), 3.93 (s, 2H), 3.20 (m, |
| | m/z = 328.1 | 3H), 2.76 (m, 1H), 2.58 (m, 2H), 2.48 (m, 1H), 1.98 (m, 1H), 1.53 (m, |
| | (M − H)⁻ | 1H) |
| 28 | LCMS (ESI−): | ¹H NMR (400 MHz, D₂O) δ 6.72-6.62 (m, 2H), 4.35 (s, 2H), 3.74- |
| | m/z = 370.1 | 3.60 (m, 1H), 3.48-3.20 (m, 2H), 2.84 (br dd, J = 5.0, 13.8 Hz, 1H), |
| | (M − H)⁻ | 2.69 (td, J = 5.6, 10.8 Hz, 1H), 2.45-2.26 (m, 1H), 1.98 (br dd, J = 6.9, |
| | | 13.8 Hz, 1H), 1.81-1.57 (m, 3H), 1.50-1.32 (m, 2H), 0.93 (t, J = 7.3 |
| | | Hz, 3H) |
| 29 | LCMS (ESI−): | ¹H NMR (400 MHz, CD₃OD) δ 6.70-6.52 (m, 2H), 4.24 (s, 2H), 3.58- |
| | m/z = 370.2 | 3.36 (m, 1H), 3.25-3.14 (m, 1H), 3.13-2.87 (m, 2H), 2.82 (dd, J = 4.9, |
| | (M − H)⁻ | 13.8 Hz, 1H), 2.71-2.48 (m, 1H), 2.43-1.93 (m, 2H), 1.64-1.27 (m, 4H), |
| | | 1.06-0.88 (m, 3H) |
| 30 | LCMS (ESI−): | ¹H NMR (400 MHz, CD₃OD) δ 6.76-6.31 (m, 2H), 4.23 (s, 2H), 3.55- |
| | m/z = 356.1 | 3.36 (m, 1H), 3.18 (dd, J = 6.9, 11.9 Hz, 1H), 3.11-2.98 (m, 1H), 2.96- |
| | (M − H)⁻ | 2.81 (m, 1H), 2.75-2.60 (m, 1H), 2.57-2.15 (m, 2H), 2.10-1.82 (m, 1H), |
| | | 1.78-1.57 (m, 1H), 1.48-1.26 (m, 1H), 1.17-0.78 (m, 3H) |

### Example 31: Synthesis of 5-(2-fluoro-6-hydroxy-4-((1-methylpiperidin-3-yl)methyl)phenyl)-1,2,5-thiadiazolidin-3-one 1,1-dioxide

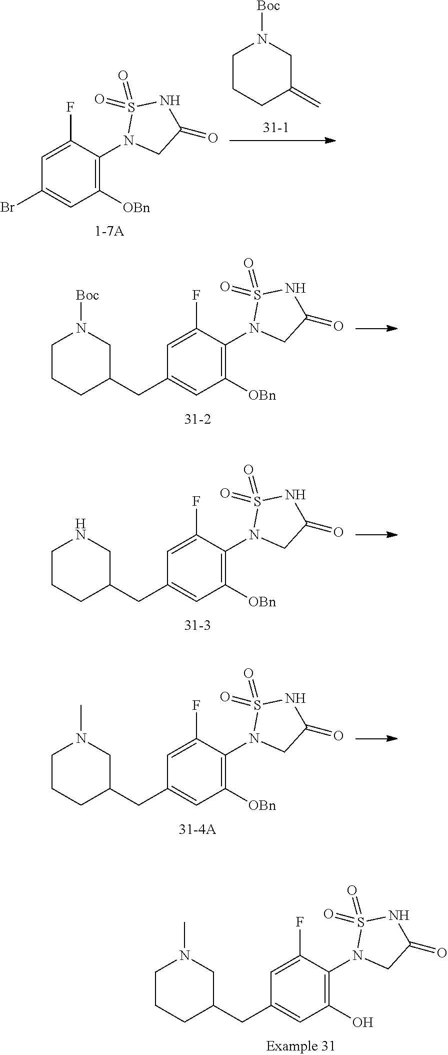 [SMILES: O=C1CN(c2c(F)cc(Br)cc2OCc2ccccc2)S(=O)(=O)N1 ; C=C1CCCN(C(=O)OC(C)(C)C)C1 ; CC(C)(C)OC(=O)N1CCCC(Cc2cc(F)c(N3CC(=O)NS3(=O)=O)c(OCc3ccccc3)c2)C1 ; O=C1CN(c2c(F)cc(CC3CCCNC3)cc2OCc2ccccc2)S(=O)(=O)N1 ; CN1CCCC(Cc2cc(F)c(N3CC(=O)NS3(=O)=O)c(OCc3ccccc3)c2)C1 ; CN1CCCC(Cc2cc(O)c(N3CC(=O)NS3(=O)=O)c(F)c2)C1]

### Step 1: Synthesis of Compound 31-2

To 31-1 (17.8 g, 90.3 mmol, 1.5 eq) was added to 0.5M 9-BBN (180 mL, 1.5 eq) then heated to 60° C. and allowed to stir for 1 h. The reaction mixture was then cooled to 20° C. and added to a solution of 1-7A (1 eq), K₂CO₃ (12.48 g, 90.31 mmol, 1.5 eq) and Pd(dppf)Cl₂—CH₂Cl₂ (4.92 g, 6.02 mmol, 0.1 eq) in DMF (250 mL) and H₂O (25 mL) then heated to 60° C. and allowed to stir for an additional 12 h. To the resulting reaction mixture was then added to H₂O (200 mL) then extracted with ethyl acetate (100 mL×3). The combined organic layers were washed with brine (300 mL), dried with Na₂SO₄, then filtered and concentrated under reduced pressure to give a crude. The crude was then purified by HPLC to give 31-2 (35 g, crude) as a brown oil. ¹H NMR (CD₃OD) δ 7.49 (d, J=7.3 Hz, 2H), 7.37-7.32 (m, 2H), 7.29 (br d, J=7.3 Hz, 1H), 6.81 (s, 1H), 6.70 (d, J=10.1 Hz, 1H), 5.19 (s, 2H), 4.33 (br s, 2H), 3.94-3.75 (m, 2H), 2.91-2.76 (m, 1H), 2.61-2.47 (m, 3H), 1.75-1.61 (m, 4H), 1.41 (br s, 9H), 1.20 (br d, J=16.9 Hz, 1H).

### Step 2: Synthesis of Compound 31-3

A mixture of 31-2 (33 g, 61.84 mmol, 1 eq) in 4M HCl/ethyl acetate (350 mL) was degassed and purged with N₂ for 3 times, then allowed to stir for 2 h at 25° C. The reaction mixture was then concentrated under reduced pressure and purified by HPLC to give 31-3 (11 g, 23.4 mmol, 37% yield) as a white solid. ¹H NMR (CD₃OD) δ 7.49 (d, J=7.3 Hz, 2H), 7.38-7.32 (m, 2H), 7.28 (s, 1H), 6.78 (s, 1H), 6.69 (dd, J=1.5, 10.1 Hz, 1H), 5.17 (s, 2H), 4.28 (s, 2H), 3.33 (br d, J=1.6 Hz, 1H), 3.23-3.15 (m, 1H), 2.86 (dt, J=3.2, 13.0 Hz, 1H), 2.68-2.60 (m, 2H), 2.59-2.51 (m, 1H), 1.98 (ddd, J=3.9, 7.6, 11.4 Hz, 1H), 1.93-1.86 (m, 1H), 1.85-1.74 (m, 1H), 1.72-1.57 (m, 1H), 1.23 (br dd, J=3.3, 12.6 Hz, 1H).

### Step 3: Synthesis of Compound 31-4A

To a solution of formaldehyde (259 mg, 3.2 mmol, 237 μL, 3 eq) in acetonitrile (5 mL) was added 31-3 (0.5 g, 1.06 mmol, 1 eq) and acetic acid (159 mg, 2.66 mmol, 152 μL, 2.5 eq) and allowed to stir for 1 h at 15° C. To this was then added NaBH₃CN (74 mg, 1.17 mmol, 1.1 eq) and allowed to stir for an additional 1 h. The resulting reaction mixture was concentrated under reduced pressure to give 31-4A (0.5 g, crude) as a yellow solid. LCMS (ESI−): m/z=446.4 (M−H)⁻.

### Step 4: Synthesis of Compound Example 31

To a solution of 31-4A (150 mg, 319 mol, 1 eq) in MeOH (5 mL) was added 5% Pd/C (100 mg) then degassed and purged with H₂ (15 psi) several times. The reaction mixture was then allowed to stir for 12 h at 15° C. The resulting reaction mixture was filtered over celite and concentrated under reduced pressure to give a crude. The crude was purified by HPLC to give Example 31 (6 mg). LCMS (ESI−): m/z=356.2 (M−H)⁻. ¹H NMR (400 MHz, CD₃OD) δ 6.59 (s, 1H), 6.56 (d, J=10.7 Hz, 1H), 4.24 (s, 2H), 3.41 (br d, J=12.3 Hz, 1H), 3.28 (br s, 1H), 2.89-2.83 (m, 1H), 2.80 (s, 3H), 2.71-2.57 (m, 2H), 2.55-2.45 (m, 1H), 2.11-2.00 (m, 1H), 1.99-1.90 (m, 1H), 1.89-1.81 (m, 1H), 1.79-1.65 (m, 1H), 1.15 (d, J=2.3 Hz, 1H).

Examples 32-54 were prepared according to the procedures described in Example 31 using the appropriate intermediates.

| Example | LCMS | ¹H NMR |
|---|---|---|
| 32 | LCMS (ESI−): | ¹H NMR (400 MHz, CD₃OD) δ 6.60-6.51 (m, 2H), 4.24 (s, 2H), 3.38 (br |
| | m/z = 412.1 | d, J = 11.5 Hz, 1H), 3.23 (br d, J = 12.1 Hz, 1H), 3.14-3.01 (m, 2H), 2.85- |
| | (M − H)⁻ | 2.66 (m, 2H), 2.64-2.54 (m, 2H), 2.53-2.45 (m, 1H), 2.21-2.07 (m, 2H), |
| | | 2.07-1.98 (m, 2H), 1.98-1.87 (m, 2H), 1.87-1.80 (m, 3H), 1.79-1.63 (m, |
| | | 1H), 1.38-1.12 (m, 1H) |
| 33 | LCMS (ESI−): | ¹H NMR (400 MHz, CD₃OD) δ 6.64-6.49 (m, 2H), 4.24 (s, 2H), 3.54-3.45 |
| | m/z = 414.1 | (m, 1H), 3.36 (br d, J = 11.8 Hz, 1H), 3.13-2.98 (m, 2H), 2.91-2.73 (m, |
| | (M − H)⁻ | 1H), 2.71-2.60 (m, 1H), 2.54 (d, J = 7.3 Hz, 2H), 2.15-1.99 (m, 1H), 1.98- |
| | | 1.79 (m, 2H), 1.79-1.61 (m, 3H), 1.48-1.29 (m, 4H), 1.27-1.14 (m, 1H), |
| | | 0.94 (t, J = 7.0 Hz, 3H) |
| 34 | LCMS (ESI+): | ¹H NMR (400 MHz, CD₃OD) δ 6.67-6.44 (m, 2H), 4.24 (s, 2H), 3.36 (br |
| | m/z = 426.1 | d, J = 11.6 Hz, 1H), 3.23 (br d, J = 11.4 Hz, 1H), 2.86 (d, J = 7.1 Hz, 2H), |
| | (M + H)⁺ | 2.62-2.53 (m, 1H), 2.52 (d, J = 7.3 Hz, 2H), 2.39 (br t, J = 11.7 Hz, 1H), |
| | | 2.20 (td, J = 8.0, 15.5 Hz, 1H), 2.04 (ttd, J = 3.7, 7.5, 15.0 Hz, 1H), 1.92- |
| | | 1.75 (m, 4H), 1.75-1.52 (m, 5H), 1.35-1.04 (m, 3H) |
| 35 | LCMS (ESI+): | ¹H NMR (400 MHz, CD₃OD) δ6.68-6.49 (m, 2H), 4.25 (s, 2H), 3.59-3.35 |
| | m/z = 428.2 | (m, 2H), 3.19-3.00 (m, 2H), 2.83 (br t, J = 11.6 Hz, 1H), 2.74-2.63 (m, |
| | (M + H)⁺ | 1H), 2.60-2.47 (m, 2H), 2.16-2.01 (m, 1H), 1.99-1.77 (m, 2H), 1.76-1.53 |
| | | (m, 3H), 1.33-1.16 (m, 1H), 0.97 (s, 9H) |
| 36 | LCMS (ESI−): | ¹H NMR (400 MHz, CD₃OD) δ 6.61-6.52 (m, 2H), 4.24 (s, 2H), 3.43 (br |
| | m/z = 426.2 | d, J = 11.5 Hz, 1H), 3.30-3.24 (m, 1H), 2.95 (br t, J = 7.7 Hz, 2H), 2.77- |
| | (M − H)⁻ | 2.65 (m, 1H), 2.59-2.48 (m, 3H), 2.10-1.98 (m, 1H), 1.91 (br d, J = 14.5 |
| | | Hz, 1H), 1.81 (br d, J = 12.7 Hz, 1H), 1.75-1.61 (m, 3H), 1.34 (br s, 6H), |
| | | 1.25-1.13 (m, 1H), 0.97-0.87 (m, 3H) |
| 37 | LCMS (ESI−): | ¹H NMR (400 MHz, D₂O) δ = 6.71-6.62 (m, 2H), 4.34 (s, 2H), 3.59 (br d, |
| | m/z = 396.1 | J = 12.4 Hz, 1H), 3.44 (br d, J = 11.0 Hz, 1H), 3.07-2.94 (m, 1H), 2.92- |
| | (M − H)⁻ | 2.77 (m, 2H), 2.73-2.45 (m, 3H), 2.14-2.01 (m, 1H), 1.95 (br d, J = 14.6 |
| | | Hz, 1H), 1.88-1.78 (m, 1H), 1.75-1.60 (m, 1H), 1.29-1.13 (m, 1H), 1.08- |
| | | 0.92 (m, 1H), 0.66 (br d, J = 7.1 Hz, 2H), 0.31 (br d, J = 4.6 Hz, 2H) |
| 38 | LCMS (ESI−): | ¹H NMR (400 MHz, D₂O) δ 6.70-6.62 (m, 2H), 4.35 (s, 2H), 3.50 (br d, |
| | m/z = 398.1 | J = 12.8 Hz, 1H), 3.34 (br d, J = 12.9 Hz, 1H), 3.06-2.98 (m, 2H), 2.86-2.72 |
| | (M − H)⁻ | (m, 1H), 2.69-2.48 (m, 3H), 2.11-1.99 (m, 1H), 1.94 (br d, J = 15.0 Hz, |
| | | 1H), 1.87-1.76 (m, 1H), 1.73-1.54 (m, 3H), 1.37-1.15 (m, 3H), 0.87 (t, J = |
| | | 7.3 Hz, 3H) |
| 39 | LCMS (ESI+): | ¹H NMR (400 MHz, D₂O) δ 7.00-6.70 (m, 2H), 6.54 (br s, 2H), 6.13 (br s, |
| | m/z = 423.2 | 1H), 4.29 (s, 2H), 4.14-4.06 (m, 1H), 4.05-3.93 (m, 1H), 3.50-3.33 (m, |
| | (M + H)⁺ | 1H), 3.25-3.08 (m, 1H), 2.73 (br t, J = 11.9 Hz, 1H), 2.62-2.53 (m, 1H), |
| | | 2.52-2.35 (m, 2H), 1.87 (br d, J = 11.6 Hz, 2H), 1.81-1.72 (m, 1H), 1.67- |
| | | 1.46 (m, 1H), 1.21-1.01 (m, 1H) |
| 40 | LCMS (ESI+): | ¹H NMR (400 MHz, D₂O) δ 6.72-6.54 (m, 2H), 4.34 (s, 2H), 4.02-3.85 |
| | m/z = 456.1 | (m, 2H), 3.49 (br d, J = 3.0 Hz, 1H), 3.41 (br t, J = 11.8 Hz, 2H), 3.36- |
| | (M + H)⁺ | 3.25 (m, 1H), 3.18-2.99 (m, 2H), 2.80 (br s, 1H), 2.61 (br dd, J = 7.4, 13.5 |
| | | Hz, 2H), 2.55-2.48 (m, 1H), 2.06 (br dd, J = 1.8, 8.0 Hz, 1H), 1.97-1.77 |
| | | (m, 2H), 1.75-1.64 (m, 1H), 1.60 (br d, J = 11.4 Hz, 4H), 1.31 (br d, J = |
| | | 6.6 Hz, 2H), 1.25 (br d, J = 11.5 Hz, 2H) |
| 41 | LCMS (ESI−): | ¹H NMR (400 MHz, CD₃OD) δ 6.63-6.50 (m, 2H), 4.24 (s, 2H), 3.58-3.46 |
| | m/z = 412.2 | (m, 1H), 3.38 (br d, J = 10.8 Hz, 1H), 3.16-3.01 (m, 2H), 2.82 (br d, J = |
| | (M − H)⁻ | 11.0 Hz, 1H), 2.74-2.62 (m, 1H), 2.55 (d, J = 7.1 Hz, 2H), 2.06 (br s, 1H), |
| | | 1.95 (br d, J = 14.3 Hz, 1H), 1.84 (br d, J = 12.9 Hz, 1H), 1.77-1.52 (m, |
| | | 4H), 1.33-1.16 (m, 1H), 0.97 (d, J = 6.3 Hz, 6H) |
| 42 | LCMS (ESI−): | ¹H NMR (400 MHz, CD₃OD) δ 7.92-7.60 (m, 2H), 6.65-6.48 (m, 2H), |
| | m/z = 422.2 | 4.32-4.09 (m, 4H), 3.54-3.36 (m, 1H), 2.88-2.72 (m, 1H), 2.69-2.42 (m, |
| | (M − H)⁻ | 3H), 2.14-1.79 (m, 3H), 1.78-1.57 (m, 1H), 1.43-1.11 (m, 2H) |
| 43 | LCMS (ESI−): | ¹H NMR (400 MHz, CD₃OD) δ 6.62-6.52 (m, 2H), 4.28-4.18 (m, 3H), |
| | m/z = 426.2 | 3.96-3.86 (m, 1H), 3.84-3.76 (m, 1H), 3.69-3.55 (m, 1H), 3.50-3.39 (m, |
| | (M − H)⁻ | 1H), 3.26-3.02 (m, 2H), 2.96-2.80 (m, 1H), 2.76-2.42 (m, 3H), 2.21-2.03 |
| | | (m, 2H), 2.01-1.69 (m, 5H), 1.63-1.46 (m, 1H), 1.32-1.17 (m, 1H) |
| 44 | LCMS (ESI−): | ¹H NMR (400 MHz, CD₃OD) δ 6.66-6.53 (m, 2H), 4.38 (s, 2H), 3.68-3.56 |
| | m/z = 439.2 | (m, 1H), 3.46-3.39 (m, 2H), 3.07 (br d, J = 6.6 Hz, 2H), 3.04-2.98 (m, |
| | (M − H)⁻ | 2H), 2.88 (dt, J = 2.7, 12.7 Hz, 1H), 2.75-2.63 (m, 2H), 2.62-2.46 (m, |
| | | 1H), 2.28-2.11 (m, 2H), 2.07-1.95 (m, 3H), 1.93-1.80 (m, 2H), 1.57-1.39 |
| | | (m, 2H), 1.30 (br s, 2H) |
| 45 | LCMS (ESI−): | ¹H NMR (400 MHz, CD₃OD) δ 7.68 (br d, J = 11.9 Hz, 1H), 7.60 (br d, |
| | m/z = 422.1 | J = 1.1 Hz, 1H), 6.57 (s, 1H), 6.54 (br d, J = 10.9 Hz, 2H), 4.23 (s, 2H), |
| | (M − H)⁻ | 4.17-3.96 (m, 2H), 3.28-3.14 (m, 2H), 2.65-2.53 (m, 2H), 2.53-2.34 (m, |
| | | 2H), 2.09-1.99 (m, 1H), 1.97-1.79 (m, 2H), 1.77-1.59 (m, 1H), 1.30-1.06 |
| | | (m, 1H) |
| 46 | LCMS (ESI−): | ¹H NMR (400 MHz, CD₃OD) δ 7.63 (d, J = 1.3 Hz, 1H), 6.62 (br d, J = |
| | m/z = 422.1 | 3.3 Hz, 1H), 6.57 (s, 1H), 6.55-6.48 (m, 2H), 4.36-4.25 (m, 2H), 4.24- |
| | (M − H)⁻ | 4.19 (m, 2H), 3.28-3.18 (m, 2H), 2.86-2.74 (m, 1H), 2.70-2.57 (m, 2H), |
| | | 2.52-2.42 (m, 1H), 2.06-1.91 (m, 2H), 1.89-1.82 (m, 1H), 1.77-1.64 (m, |
| | | 1H), 1.36-1.11 (m, 2H) |
| 47 | LCMS (ESI−): | ¹H NMR (400 MHz, CD₃OD) δ 8.25 (s, 1H), 8.12 (s, 1H), 6.61-6.51 (m, |
| | m/z = 423.1 | 2H), 4.27 (s, 2H), 4.25 (s, 2H), 3.57-3.47 (m, 1H), 3.41 (br d, J = 10.9 Hz, |
| | (M − H)⁻ | 1H), 3.03-2.90 (m, 1H), 2.81-2.70 (m, 1H), 2.63-2.46 (m, 2H), 2.16-2.03 |
| | | (m, 1H), 1.96 (br d, J = 14.5 Hz, 1H), 1.83 (br d, J = 13.4 Hz, 1H), 1.74 |
| | | (br d, J = 13.0 Hz, 1H), 1.35-1.15 (m, 1H) |
| 48 | LCMS (ESI−): | ¹H NMR (400 MHz, CD₃OD) δ 8.34 (s, 1H), 7.37 (s, 1H), 6.66-6.43 (m, |
| | m/z = 423.1 | 2H), 4.51-4.30 (m, 2H), 4.23 (s, 2H), 3.51-3.32 (m, 2H), 2.95-2.78 (m, |
| | (M − H)⁻ | 1H), 2.77-2.55 (m, 2H), 2.54-2.41 (m, 1H), 2.12-1.92 (m, 2H), 1.90-1.82 |
| | | (m, 1H), 1.75-1.64 (m, 1H), 1.32-1.19 (m, 1H) |
| 49 | LCMS (ESI−): | ¹H NMR (400 MHz, D₂O) δ 6.65 (s, 2H), 4.34 (s, 2H), 3.49 (br d, J = 11.7 |
| | m/z = 384.1 | Hz, 1H), 3.34 (br d, J = 12.2 Hz, 1H), 3.04-2.92 (m, 2H), 2.85-2.74 (m, |
| | (M − H)⁻ | 1H), 2.69-2.48 (m, 3H), 2.09-1.99 (m, 1H), 1.93 (br d, J = 14.5 Hz, 1H), |
| | | 1.86-1.76 (m, 1H), 1.73-1.53 (m, 3H), 1.28-1.10 (m, 1H), 0.89 (t, J = 7.3 |
| | | Hz, 3H) |
| 50 | LCMS (ESI−): | ¹H NMR (400 MHz, D₂O) δ 6.57-6.42 (m, 2H), 4.32 (s, 2H), 3.55-3.39 |
| | m/z = 396.1 | (m, 1H), 3.36-3.25 (m, 1H), 2.96-2.73 (m, 3H), 2.70-2.41 (m, 3H), 2.17- |
| | (M − H) | 1.99 (m, 2H), 1.95-1.78 (m, 2H), 1.71 (br d, J = 13.4 Hz, 1H), 1.31-1.13 |
| | | (m, 1H), 0.94-0.89 (m, 6H) |
| 51 | LCMS (ESI−): | ¹H NMR (400 MHz, D₂O) δ 6.64 (br s, 2H), 4.33 (s, 2H), 3.48 (br d, J = |
| | m/z = 372.1 | 11.8 Hz, 1H), 3.33 (br d, J = 11.6 Hz, 1H), 3.08 (q, J = 7.1 Hz, 2H), 2.75 |
| | (M − H)⁻ | (br t, J = 12.6 Hz, 1H), 2.64-2.56 (m, 1H), 2.56-2.47 (m, 2H), 2.08-1.86 |
| | | (m, 2H), 1.77 (br d, J = 13.0 Hz, 1H), 1.70-1.53 (m, 1H), 1.23 (br t, J = |
| | | 7.3 Hz, 3H), 1.19-1.10 (m, 1H) |
| 52 | LCMS (ESI−): | ¹H NMR (400 MHz, CD₃OD) δ 7.32 (s, 2H), 6.58-6.44 (m, 2H), 4.26-4.20 |
| | m/z = 422.2 | (m, 2H), 4.04 (br s, 2H), 3.10 (br d, J = 9.3 Hz, 1H), 2.94 (br d, J = 10.8 |
| | (M − H) | Hz, 1H), 2.62-2.51 (m, 2H), 2.50-2.40 (m, 1H), 2.27 (br t, J = 10.6 Hz, |
| | | 1H), 1.98 (br s, 1H), 1.89-1.76 (m, 2H), 1.74-1.63 (m, 1H), 1.23-1.08 (m, 1H) |
| 53 | LCMS (ESI−): | ¹H NMR (400 MHz, CD₃OD) δ 7.73 (d, J = 2.3 Hz, 1H), 6.57-6.49 (m, |
| | m/z = 422.2 | 2H), 6.45 (br d, J = 0.8 Hz, 1H), 4.35-4.27 (m, 2H), 4.23 (s, 2H), 3.53- |
| | (M − H)⁻ | 3.35 (m, 2H), 3.00-2.82 (m, 1H), 2.72-2.58 (m, 2H), 2.47 (dd, J = 7.9, |
| | | 13.5 Hz, 1H), 2.11-2.00 (m, 1H), 1.99-1.93 (m, 1H), 1.87 (br d, J = 12.5 |
| | | Hz, 1H), 1.73 (br dd, J = 1.8, 3.4 Hz, 1H), 1.32-1.18 (m, 1H) |
| 54 | LCMS (ESI−): | ¹H NMR (DMSO-d₆) δ 9.13-9.54 (m, 1 H) 8.01-8.42 (m, 1 H) 6.48-6.60 |
| | m/z = 342.2 | (m, 2 H) 3.93 (s, 2 H) 3.20 (br d, J = 12.51 Hz, 1 H) 3.02-3.11 (m, 1 H) |
| | (M − H)⁻ | 2.69-2.78 (m, 1 H) 2.53-2.60 (m, 1 H) 2.38-2.46 (m, 2 H) 1.81-1.97 (m, 1 |
| | | H) 1.68-1.80 (m, 2 H) 1.47-1.60 (m, 1 H) 1.09-1.27 (m, 1 H) |

### Example 55: Synthesis of 5-(2-fluoro-6-hydroxy-4-((1-methylazepan-3-yl)methyl)phenyl)-1,2,5-thiadiazolidin-3-one 1,1-dioxide

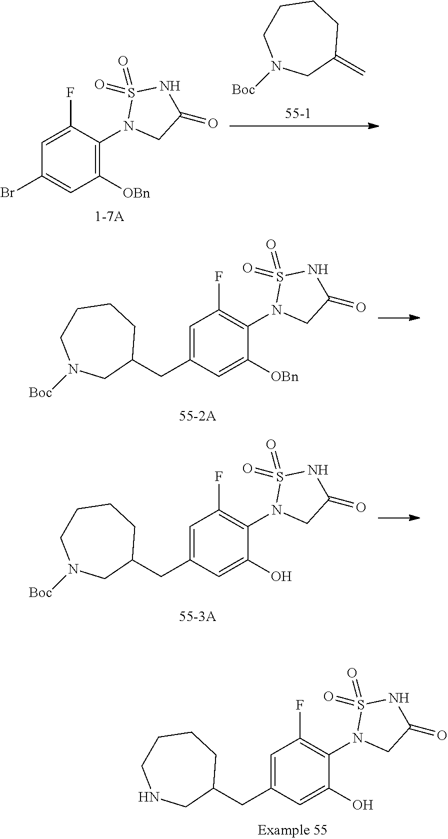 [SMILES: O=C1CN(c2c(F)cc(Br)cc2OCc2ccccc2)S(=O)(=O)N1 ; C=C1CCCCN(C(=O)OC(C)(C)C)C1 ; CC(C)(C)OC(=O)N1CCCCC(Cc2cc(F)c(N3CC(=O)NS3(=O)=O)c(OCc3ccccc3)c2)C1 ; CC(C)(C)OC(=O)N1CCCCC(Cc2cc(O)c(N3CC(=O)NS3(=O)=O)c(F)c2)C1 ; O=C1CN(c2c(O)cc(CC3CCCCNC3)cc2F)S(=O)(=O)N1]

### Step 1: Synthesis of Compound 55-2A

To a solution of 0.5M 9-BBN (3.18 mL, 1.5 eq) was added 55-1 (244 mg, 1.16 mmol, 1.2 eq) then heated to 60° C. and allowed to stir 1 h. The reaction mixture was then allowed to cool to 20° C. To this was then added a solution of 1-7A (400 mg, 963 μmol, 1 eq), K₂CO₃ (200 mg, 1.44 mmol, 1.5 eq) and Pd(dppf)Cl₂—CH₂Cl₂ (78 mg, 96 μmol, 0.1 eq) in DMF (4 mL) and H₂O (0.4 mL) then heated to 60° C. and allowed to stir for an additional 12 h. To the reaction mixture was added H₂O (10 mL) then extracted with ethyl acetate (10 mL×3). The combined organic layers were washed with brine (20 mL), dried with Na₂SO₄, then filtered and concentrated under reduced pressure to give 55-2A (500 mg, 913 μmol, 94% yield) as a yellow oil. LCMS (ESI−): m/z=546.4 (M−H)⁻. ¹H NMR (400 MHz, CD₃OD) δ 7.49 (s, 2H), 7.38-7.33 (m, 2H), 7.29 (s, 1H), 6.75 (s, 1H), 6.66 (dd, J=1.4, 10.2 Hz, 1H), 5.19 (s, 2H), 4.33 (s, 2H), 3.61-3.46 (m, 1H), 3.43-3.34 (m, 2H), 3.20-3.05 (m, 1H), 2.53 (s, 2H), 1.91-1.57 (m, 5H), 1.45 (d, J=6.8 Hz, 9H), 1.38-1.28 (m, 1H), 1.20-1.06 (m, 1H)

### Step 2: Synthesis of Compound 55-3A

To a mixture 55-2A (470 mg, 858 μmol, 1 eq) in THE (10 mL) was added 10% Pd/C (20 mg) under N₂. The suspension was degassed under vacuum and purged with H₂ (15 psi) 3 times then allowed to stir for 2 h at 25° C. The resulting reaction mixture was filtered over celite and then concentrated under reduced pressure to give 55-3A (370 mg, 808 μmol, 94% yield) as a colorless oil. LCMS (ESI−): m/z 456.3 (M−H)⁻.

### Step 3: Synthesis of Compound Example 55

To a solution of 55-3 (370 mg, 808 μmol, 1 eq) in ethyl acetate (2 mL) added 4M HCl/ethyl acetate (20 mL) and allowed to stir for 20 min at 25° C. The reaction mixture was concentrated under reduced pressure to give a crude. The crude was then purified by HPLC to give Example 55 (94 mg, 263 μmol, 32% yield). LCMS (ESI+): m/z=358.1 (M+H)⁺. ¹H NMR (400 MHz, CD₃OD) δ 6.66-6.47 (m, 2H), 4.23 (s, 2H), 3.22-3.11 (m, 3H), 2.86 (dd, J=10.3, 13.6 Hz, 1H), 2.67-2.58 (m, 1H), 2.54-2.45 (m, 1H), 2.21-2.05 (m, 1H), 1.97-1.75 (m, 4H), 1.62-1.50 (m, 1H), 1.47-1.34 (m, 1H).

### Example 56: Synthesis of 5-(2-fluoro-6-hydroxy-4-((1-methylazepan-3-yl)methyl)phenyl)-1,2,5-thiadiazolidin-3-one 1,1-dioxide

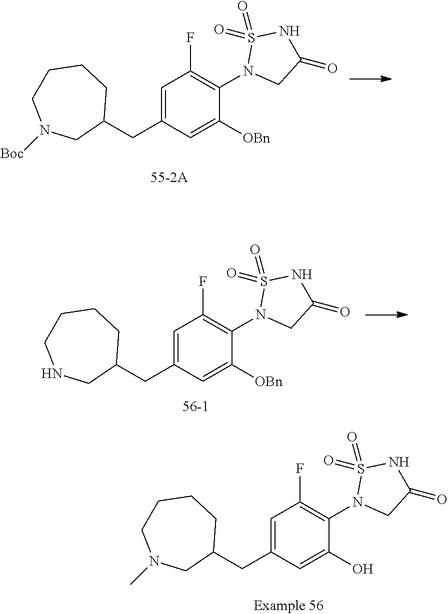 [SMILES: CC(C)(C)OC(=O)N1CCCCC(Cc2cc(F)c(N3CC(=O)NS3(=O)=O)c(OCc3ccccc3)c2)C1 ; O=C1CN(c2c(F)cc(CC3CCCCNC3)cc2OCc2ccccc2)S(=O)(=O)N1 ; CN1CCCCC(Cc2cc(O)c(N3CC(=O)NS3(=O)=O)c(F)c2)C1]

Compound 56-1 was prepared according to the procedure described for Example 55. To a solution of 56-1 (50 mg, 140 μmol, 1 eq) and formaldehyde (218 mg, 2.7 mmol, 0.2 mL, 19.2 eq) in MeOH (20 mL) was added 10% Pd/C (20 mg, 139 μmol) under N₂. The suspension was degassed under vacuum and purged with H₂ (15 psi) 3 times then allowed to stir for 2 h at 20° C. The resulting reaction mixture was filtered over celite and then concentrated under reduced pressure to give a crude. The crude was then purified by HPLC to give Example 56 (2 mg, 5.2 μmol, 3% yield). LCMS (ESI−): m/z=370.1 (M−H)⁻. ¹H NMR (400 MHz, CD₃OD) δ 6.66-6.52 (m, 2H), 4.24 (s, 2H), 3.30-3.06 (m, 4H), 2.83 (s, 3H), 2.66-2.48 (m, 2H), 2.24 (br d, J=7.4 Hz, 1H), 2.02-1.83 (m, 4H), 1.67-1.58 (m, 1H), 1.52-1.38 (m, 1H).

Examples 57-60 were prepared according to the procedures described in Examples 55 and 56 using the appropriate intermediates.

| Example | LCMS | ¹H NMR |
|---|---|---|
| 57 | LCMS (ESI−): | ¹H NMR (400 MHz, D₂O) δ 6.64 (br t, J = 4.8 Hz, 2H), 4.34 (s, 2H), |
| | m/z = 356.1 | 3.31-3.26 (m, 2H), 3.18-3.06 (m, 2H), 2.53 (br dd, J = 7.1, 11.4 Hz, 2H), |
| | (M − H)⁻ | 1.99-1.81 (m, 4H), 1.72 (br d, J = 13.3 Hz, 1H), 1.60-1.45 (m, 1H), 1.36- |
| | | 1.21 (m, 1H) |
| 58 | LCMS (ESI−): | ¹H NMR (400 MHz, D₂O) δ 6.64-6.53 (m, 2H), 4.29 (s, 2H), 3.32-2.94 |
| | m/z = 356.1 | (m, 4H), 2.57-2.37 (m, 2H), 1.96-1.75 (m, 4H), 1.73-1.60 (m, 1H), 1.55- |
| | (M − H)⁻ | 1.41 (m, 1H), 1.30-1.15 (m, 1H) |
| 59 | LCMS (ESI−): | ¹H NMR (400 MHz, CD₃OD) δ 6.63-6.48 (m, 2H), 4.23 (s, 2H), 3.30-3.21 |
| | m/z = 356.1 | (m, 2H), 3.20-3.02 (m, 2H), 2.63-2.46 (m, 2H), 2.03-1.86 (m, 4H), 1.85- |
| | (M − H)⁻ | 1.70 (m, 1H), 1.61-1.48 (m, 1H), 1.41-1.27 (m, 1H) |
| 60 | LCMS (ESI−): | ¹H NMR (400 MHz, CD₃OD) δ 6.60-6.50 (m, 2H), 4.23 (s, 2H), 2.85 (s, |
| | m/z = 372.1 | 3H), 2.62-2.46 (m, 2H), 2.11-1.73 (m, 6H), 1.67-1.57 (m, 1H), 1.46-1.23 |
| | (M − H)⁻ | (m, 4H) |

### Example 61: Synthesis of 5-(2-fluoro-6-hydroxy-4-(2-(1-methylpyrrolidin-3-yl)ethyl)phenyl)-1,2,5-thiadiazolidin-3-one 1,1-dioxide

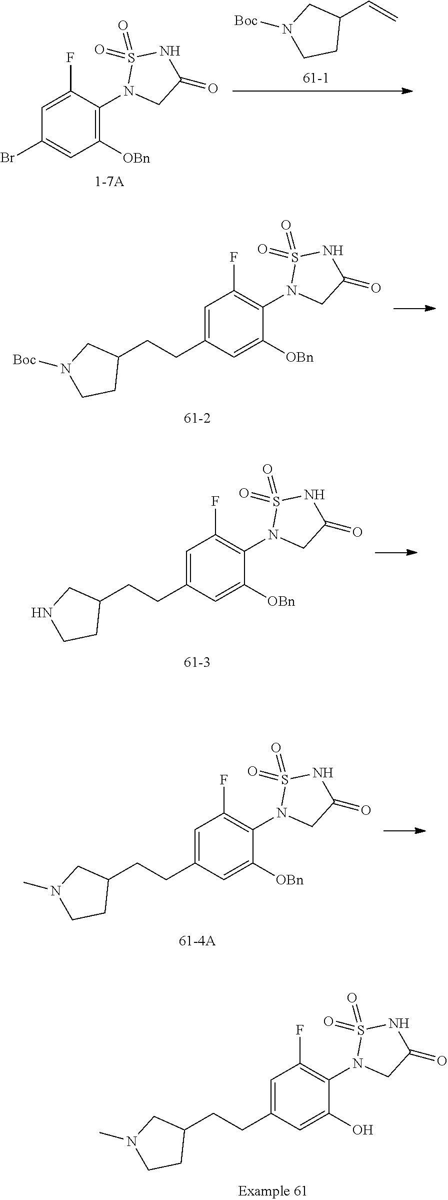 [SMILES: O=C1CN(c2c(F)cc(Br)cc2OCc2ccccc2)S(=O)(=O)N1 ; C=CC1CCN(C(=O)OC(C)(C)C)C1 ; CC(C)(C)OC(=O)N1CCC(CCc2cc(F)c(N3CC(=O)NS3(=O)=O)c(OCc3ccccc3)c2)C1 ; O=C1CN(c2c(F)cc(CCC3CCNC3)cc2OCc2ccccc2)S(=O)(=O)N1 ; CN1CCC(CCc2cc(F)c(N3CC(=O)NS3(=O)=O)c(OCc3ccccc3)c2)C1 ; CN1CCC(CCc2cc(O)c(N3CC(=O)NS3(=O)=O)c(F)c2)C1]

### Step 1: Synthesis of Compound 61-2

To a solution of 0.5M 9-BBN (14.4 mL, 1.5 eq) was added 61-1 (1.4 g, 7.2 mmol, 1.5 eq) then heated to 60° C. and allowed to stir 1 h. The reaction mixture was then allowed to cool to 20° C. To this was then added a solution of 1-7A (2 g, 4.82 mmol, 1 eq), K₂CO₃ (998 mg, 7.2 mmol, 1.5 eq) and Pd(dppf)Cl₂—CH₂Cl₂ (393 mg, 481 μmol, 0.1 eq) in DMF (20 mL) and H₂O (10 mL) then heated to 60° C. and allowed to stir for an additional 12 h. To the reaction mixture was added H₂O (50 mL) then extracted with ethyl acetate (50 mL×3). The combined organic layers were washed with brine (50 mL), dried with Na₂SO₄, then filtered and concentrated under reduced pressure to give a crude. The crude was then purified by HPLC to give 61-2 (1.5 g, 2.8 mmol, 58% yield) as a white solid.

### Step 2: Synthesis of Compound 61-3

A solution of 61-2 (470 mg, 880 μmol, 1 eq) in 4M HCl/ethyl acetate (10 mL) was allowed to stir for 0.5 h at 25° C. The reaction mixture was concentrated under reduced pressure to give 61-3 (400 mg, crude) as a yellow solid.

### Step 3: Synthesis of Compound 61-4A Series

To a solution of 61-3 (100 mg, 230 μmol, 1 eq), formaldehyde (14 mg, 461 μmol, 13 μL, 2 eq) in acetonitrile (2 mL) was added acetic acid (34 mg, 576 μmol, 33 μL, 2.5 eq) and allowed to stir for 0.5 h at 25° C. To this was then added NaBH₃CN (29 mg, 461 μmol, 2 eq) and allowed to stir for an additional 1 h. To the reaction mixture was added H₂O (5 mL) then extracted with ethyl acetate (5 mL×3). The combined organic layers were washed with brine (10 mL), dried with Na₂SO₄, then filtered and concentrated under reduced pressure to give 61-4A (70 mg, crude) as a white solid. LCMS (ESI+): m/z=448.3 (M+H)+.

### Step 4: Synthesis of Compound Example 61

To a solution of 61-4A (70 mg, 156 μmol, 1 eq) in MeOH (3 mL) was added 10% Pd/C (50 mg) under N₂. The suspension was degassed under vacuum and purged with H₂ (15 psi) 3 times then allowed to stir for 12 h at 25° C. The resulting reaction mixture was filtered over celite and then concentrated under reduced pressure to give a crude. The crude was then purified by HPLC to give Example 61 (6 mg, 15 μmol, 10% yield). LCMS (ESI−): m/z=356.1 (M−H)⁻. ¹H NMR (D₂O) δ 6.58-6.45 (m, 2H), 4.33 (s, 2H), 3.46-3.26 (m, 2H), 2.93-2.80 (m, 4H), 2.65-2.49 (m, 2H), 2.41-2.16 (m, 2H), 1.81-1.64 (m, 3H), 1.22 (t, J 7.3 Hz, 1H).

Example 62-64 were prepared according to the procedures described in Example 61 using the appropriate intermediates.

| Example | LCMS | ¹H NMR |
|---|---|---|
| 62 | LCMS (ESI−): | ¹H NMR (400 MHz, CD₃CD₂OD) δ 6.65-6.57 (m, 2H), 4.32 (s, 2H), |
| | m/z = 342.1 | 3.43-3.36 (m, 2H), 3.26-3.16 (m, 1H), 2.81 (dd, J = 9.3, 11.2 Hz, 1H), |
| | (M − H)⁻ | 2.71-2.56 (m, 2H), 2.33-2.17 (m, 2H), 1.83-1.75 (m, 2H), 1.70-1.60 |
| | | (m, 1H) |
| 63 | LCMS (ESI−): | ¹H NMR (400 MHz, D₂O) δ 6.66 (br s, 2H), 4.34 (s, 2H), 3.58-3.09 (m, |
| | m/z = 398.1 | 3H), 3.07-2.73 (m, 3H), 2.71-2.48 (m, 2H), 2.37-2.14 (m, 2H), 1.99 (td, |
| | (M − H)⁻ | J = 6.8, 13.8 Hz, 1H), 1.83-1.59 (m, 3H), 0.94 (d, J = 6.6 Hz, 6H) |
| 64 | LCMS (ESI−): | ¹H NMR (400 MHz, D₂O) δ 6.71-6.65 (m, 2H), 4.34 (s, 2H), 3.81-3.37 |
| | m/z = 412.2 | (m, 2H), 3.31-2.91 (m, 4H), 2.69-2.53 (m, 2H), 2.44-2.04 (m, 2H), 1.89- |
| | (M − H)⁻ | 1.41 (m, 6H), 0.87 (d, J = 6.4 Hz, 6H) |

### Example 65: Synthesis of 5-(2-fluoro-6-hydroxy-4-(2-(1-methylpyrrolidin-3-yl)ethyl)phenyl)-1,2,5-thiadiazolidin-3-one 1,1-dioxide

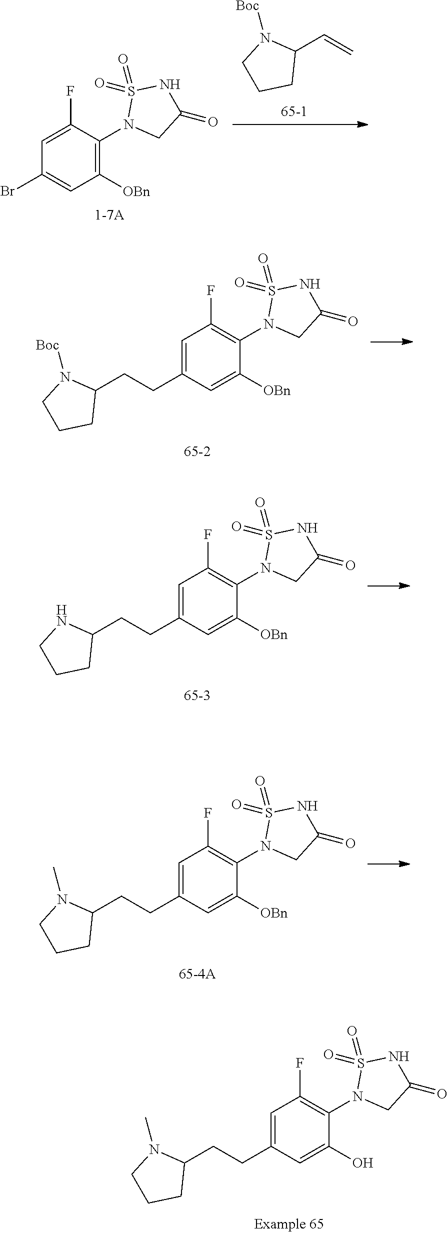 [SMILES: O=C1CN(c2c(F)cc(Br)cc2OCc2ccccc2)S(=O)(=O)N1 ; C=CC1CCCN1C(=O)OC(C)(C)C ; CC(C)(C)OC(=O)N1CCCC1CCc1cc(F)c(N2CC(=O)NS2(=O)=O)c(OCc2ccccc2)c1 ; O=C1CN(c2c(F)cc(CCC3CCCN3)cc2OCc2ccccc2)S(=O)(=O)N1 ; CN1CCCC1CCc1cc(F)c(N2CC(=O)NS2(=O)=O)c(OCc2ccccc2)c1 ; CN1CCCC1CCc1cc(O)c(N2CC(=O)NS2(=O)=O)c(F)c1]

### Step 1: Synthesis of Compound 65-2

To a solution of 0.5M 9-BBN (3.61 mL, 1.5 eq) was added 65-1 (356 mg, 1.8 mmol, 1.5 eq) then heated to 60° C. and allowed to stir 1 h. The reaction mixture was then allowed to cool to 20° C. To this was then added a solution of 1-7A (500 mg, 1.20 mmol, 1 eq), K₂CO₃ (249 mg, 1.81 mmol, 1.5 eq) and Pd(dppf)Cl₂—CH₂Cl₂ (98 mg, 120 μmol, 0.1 eq) in DMF (5 mL) and H₂O (0.5 mL) then heated to 60° C. and allowed to stir for an additional 12 h. To the reaction mixture was added H₂O (50 mL) then extracted with ethyl acetate (50 mL×3). The combined organic layers were washed with brine (50 mL), dried with Na₂SO₄, then filtered and concentrated under reduced pressure to give a crude. The crude was then purified by HPLC to give 65-2 (230 mg, 431 μmol, 35% yield) as a yellow solid.

### Step 2: Synthesis of Compound 65-3

A solution of 65-2 (230 mg, 431 μmol, 1 eq) in 4M HCl/ethyl acetate (10 mL) was allowed to stir for 0.5 h at 25° C. The reaction mixture was concentrated under reduced pressure to give 65-3 (250 mg, crude) as a white solid.

### Step 3: Synthesis of Compound 65-4A

To a solution of 65-3 (100 mg, 213 μmol, 1 eq), formaldehyde (34 mg, 425 μmol, 31 μL, 2 eq) in 1,2-dichloroethane (2 mL) was added diisopropylethylamine (30 mg, 234 μmol, 40 μL, 1.1 eq) and allowed to stir for 0.5 h at 25° C. To this was then added NaBH(OAc)₃ (139 mg, 638 μmol, 3 eq) and allowed to stir for an additional 1 h. To the reaction mixture was added H₂O (5 mL) then extracted with ethyl acetate (5 mL×3). The combined organic layers were washed with brine (10 mL), dried with Na₂SO₄, then filtered and concentrated under reduced pressure to give 65-4A (70 mg, crude) as a white solid. LCMS (ESI−): m/z 446.4 (M−H)⁻.

### Step 4: Synthesis of Compound Example 65

To a solution of 65-4A (20 mg, 44 μmol, 1 eq) in MeOH (3 mL) was added 10% Pd/C (10 mg) under N₂. The suspension was degassed under vacuum and purged with H₂ (15 psi) 3 times then allowed to stir for 12 h at 25° C. The resulting reaction mixture was filtered over celite and then concentrated under reduced pressure to give a crude. The crude was then purified by HPLC to give Example 65 (2 mg, 5.4 μmol, 12% yield). LCMS (ESI−): m/z=356.1 (M−H)⁻. ¹H NMR (400 MHz, D₂O) δ 6.75-6.66 (m, 2H), 4.34 (s, 2H), 3.68-3.57 (m, 1H), 3.26-3.15 (m, 1H), 3.07 (br d, J=9.3 Hz, 1H), 2.83 (s, 3H), 2.76 (ddd, J=5.6, 8.6, 14.1 Hz, 1H), 2.67-2.55 (m, 1H), 2.39-2.17 (m, 2H), 2.12-1.69 (m, 4H).

Examples 66-68 were prepared according to the procedures described in Example 65 using the appropriate intermediates.

| Example | LCMS | ¹H NMR |
|---|---|---|
| 66 | LCMS (ESI−): | ¹H NMR (400 MHz, D₂O) δ 6.69 (br t, J = 4.8 Hz, 2H), 4.34 (s, 2H), |
| | m/z = 342.1 | 3.53-3.42 (m, 1H), 3.34-3.19 (m, 2H), 2.80-2.60 (m, 2H), 2.24-2.14 (m, |
| | (M − H)⁻ | 1H), 2.11-1.87 (m, 4H), 1.65 (br dd, J = 8.9, 13.0 Hz, 1H) |
| 67 | LCMS (ESI+): | ¹H NMR (400 MHz, D₂O) δ = 6.74-6.68 (m, 2H), 4.34 (s, 2H), 3.59 (br s, |
| | m/z = 414.1 | 1H), 3.32-3.15 (m, 2H), 3.12-2.91 (m, 2H), 2.79 (br dd, J = 6.2, 13.6 Hz, |
| | (M + H)⁻ | 1H), 2.65-2.51 (m, 1H), 2.38-2.13 (m, 2H), 2.11-1.95 (m, 2H), 1.92-1.69 |
| | | (m, 2H), 1.64-1.34 (m, 3H), 0.85 (d, J = 6.5 Hz, 6H) |
| 68 | LCMS (ESI+): | ¹H NMR (400 MHz, D₂O) δ 6.74-6.67 (m, 2H), 4.34 (s, 2H), 3.71-3.60 |
| | m/z = 400.1 | (m, 1H), 3.26 (br d, J = 8.0 Hz, 1H), 3.14-2.98 (m, 2H), 2.87-2.72 (m, |
| | (M + H)⁻ | 2H), 2.65-2.51 (m, 1H), 2.39-2.13 (m, 2H), 2.12-1.84 (m, 4H), 1.82-1.68 |
| | | (m, 1H), 0.94 (d, J = 6.6 Hz, 6H) |

### Example 69: Synthesis of 5-(4-(azetidin-3-ylmethyl)-2-fluoro-6-hydroxyphenyl)-1,2,5-thiadiazolidin-3-one 1,1-dioxide

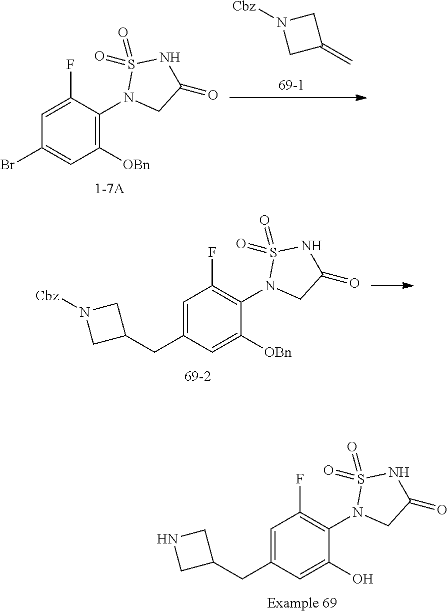 [SMILES: O=C1CN(c2c(F)cc(Br)cc2OCc2ccccc2)S(=O)(=O)N1 ; C=C1CN(C(=O)OCc2ccccc2)C1 ; O=C1CN(c2c(F)cc(CC3CN(C(=O)OCc4ccccc4)C3)cc2OCc2ccccc2)S(=O)(=O)N1 ; O=C1CN(c2c(O)cc(CC3CNC3)cc2F)S(=O)(=O)N1]

### Step 1: Synthesis of Compound 69-2

To a solution of 0.5M 9-BBN (3.18 mL, 1.65 eq) was added 69-1 (293 mg, 1.4 mmol, 1.5 eq) then heated to 60° C. and allowed to stir 1 h. The reaction mixture was then allowed to cool to 20° C. To this was then added a solution of 1-7A (400 mg, 963 μmol, 1 eq), K₂CO₃ (200 mg, 1.4 mmol, 1.5 eq) and Pd(dppf)Cl₂—CH₂Cl₂ (78.67 mg, 96.33 μmol, 0.1 eq) in DMF (4 mL) and H₂O (0.4 mL) then heated to 60° C. and allowed to stir for an additional 12 h. To the reaction mixture was added H₂O (10 mL) then extracted with ethyl acetate (10 mL×3). The combined organic layers were washed with brine (10 mL), dried with Na₂SO₄, then filtered and concentrated under reduced pressure to give a crude. The crude was then purified by HPLC to give 69-2 (150 mg, 278 μmol, 28% yield) as a red solid.

### Step 2: Synthesis of Compound Example 69

To a solution of 69-2 (130 mg, 240 μmol, 1 eq) in THE (10 mL) was added 10% Pd/C (50 mg) under N₂. The suspension was degassed under vacuum and purged with H₂ (15 psi) 3 times then allowed to stir for 2 h at 25° C. The resulting reaction mixture was filtered over celite and then concentrated under reduced pressure to give a crude. The crude was then purified by HPLC to give Example 69 (3.6 mg, 11.4 μmol, 5% yield). ¹H NMR (400 MHz, CD₃OD) δ 6.61-6.52 (m, 2H), 4.22 (s, 2H), 4.09-4.02 (m, 2H), 3.88-3.79 (m, 2H), 3.27-3.16 (m, 1H), 2.89 (d, J=7.8 Hz, 2H).

### Example 70: Synthesis of 5-(2-fluoro-6-hydroxy-4-((2-propylpyrrolidin-1-yl)methyl)phenyl)-1,2,5-thiadiazolidin-3-one 1,1-dioxide

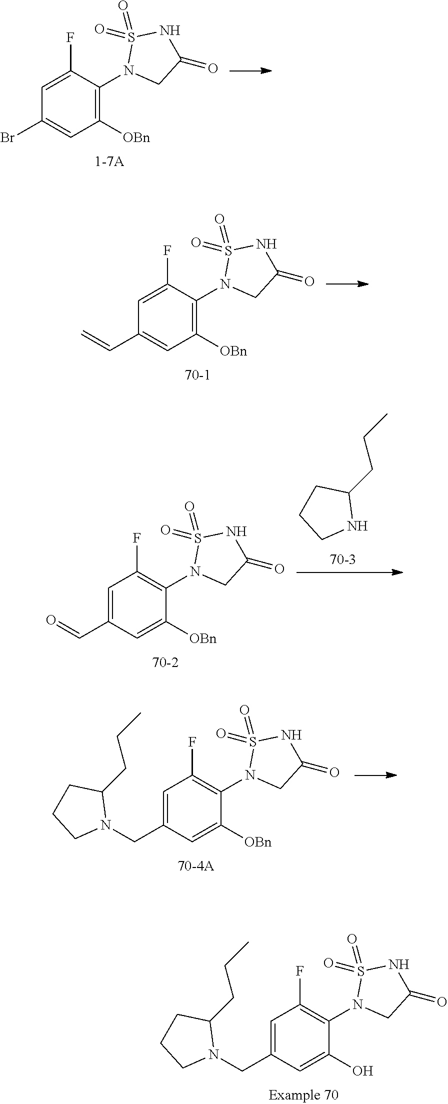

### Step 1: Synthesis of Compound 70-1

To a solution of 1-7A (2 g, 4.82 mmol, 1 eq), 4,4,5,5-tetramethyl-2-vinyl-1,3,2-dioxaborolane (7.42 g, 48.17 mmol, 8.17 mL, 10 eq) and K₂CO₃ (2.0 g, 14.4 mmol, 3 eq) in DMF (20 mL) and H₂O (2 mL) was added Pd(dppf)Cl₂—CH₂Cl₂ (393 mg, 481 μmol, 0.1 eq) then heated to 80° C. and allowed to stir for 12 h. To the reaction mixture was added H₂O (30 mL) then extracted with ethyl acetate (30 mL×3). The combined organic layers were washed with brine (40 mL), dried with Na₂SO₄, then filtered and concentrated under reduced pressure to give a crude. The crude was then purified by column chromatography (SiO₂, ethyl acetate/methanol-100/1 to 5/1) to give 70-1 (1.4 g, 3.86 mmol, 80% yield) as a yellow oil. ¹H NMR (400 MHz, CD₃OD) δ 7.53 (br d, J=7.5 Hz, 2H), 7.38-7.32 (m, 2H), 7.31-7.24 (m, 1H), 7.00 (s, 1H), 7.03-6.96 (m, 1H), 6.91 (br d, J=10.6 Hz, 1H), 6.68 (br dd, J=10.8, 17.3 Hz, 1H), 5.81 (br d, J=17.6 Hz, 1H), 5.32 (br d, J=10.9 Hz, 1H), 5.20 (s, 2H), 4.28 (s, 2H).

### Step 2: Synthesis of Compound 70-2

To a solution of 70-1 (1.4 g, 3.86 mmol, 1 eq) and 2,6-dimethylpyridine (827 mg, 7.7 mmol, 900 μL, 2 eq) in 1,4-dioxane (40 mL) and H₂O (10 mL) was added NaIO₄ (3.31 g, 15.45 mmol, 856 μL, 4 eq) slowly and allowed to stir for 2 h at 0° C. under N₂. To this was then added potassium osmate (VI) dihydrate (71 mg, 193 μmol, 0.05 eq) and allowed to warm to 20° C. and stirred for an additional 10 h. To the reaction mixture was added sat. NaHCO_{3(aq)} (100 mL) then extracted with ethyl acetate (100 mL×3). The combined organic layers were washed with brine (50 mL), dried with Na₂SO₄, then filtered and concentrated under reduced pressure to give a crude. The crude was then purified by prep-TLC (CH₂Cl₂/methanol-5:1, Rf=0.13) to give 70-2 (800 mg, 2.2 mmol, 56% yield) as a white solid. ¹H NMR (400 MHz, CD₃OD) δ 9.90 (d, J=1.5 Hz, 1H), 7.57 (d, J=7.9 Hz, 7H), 5.28 (s, 2H), 4.33 (s, 2H).

### Step 3: Synthesis of Compound 70-4A

To a solution of 70-2 (100 mg, 274 μmol, 1 eq) in CH₂Cl₂ (4 mL) was added diisopropylethylamine (35 mg, 274 μmol, 47 μL, 1 eq) and 4A MS (100 mg) at 25° C. then was allowed to stir for 0.5 h. To this was then added 70-3 (49 mg, 329 μmol, 1.2 eq) and allowed to stir for an additional 2 h. To the reaction mixture was then added NaBH(OAc)₃ (174 mg, 823 μmol, 3 eq) and allowed to stir for an additional 12 h. To the reaction mixture was added H₂O (5 mL) then extracted with CH₂Cl₂ (5 mL×3). The combined organic layers were washed with brine (10 mL), dried with Na₂SO₄, then filtered and concentrated under reduced pressure to give 70-4A (110 mg, crude) as a yellow oil. LCMS (ESI−): m/z=460.1 (M−H)⁻.

### Step 4: Synthesis of Compound Example 70

To a solution of 70-4A (100 mg, 216 μmol, 1 eq) in THE (20 mL) was added 10% Pd/C (50 mg) under N₂. The suspension was degassed under vacuum and purged with H₂ (15 psi) 3 times then allowed to stir for 2 h at 25° C. The resulting reaction mixture was filtered over celite and then concentrated under reduced pressure to give a crude. The crude was then purified by HPLC to give Example 70 (5 mg, 13 μmol, 6% yield). LCMS (ESI−): m/z=370.1 (M−H)⁻. ¹H NMR (400 MHz, CD₃OD) δ 7.00-6.69 (m, 2H), 4.44 (br d, J=12.9 Hz, 1H), 4.26 (s, 2H), 4.04 (br d, J=12.9 Hz, 1H), 3.48-3.35 (m, 2H), 3.24-3.10 (m, 1H), 2.41-2.28 (m, 1H), 2.16-2.03 (m, 1H), 2.03-1.92 (m, 1H), 1.91-1.83 (m, 1H), 1.81-1.70 (m, 1H), 1.60-1.29 (m, 3H), 1.06-0.93 (m, 3H).

Example 71-73 were prepared according to the procedures described in Example 70 using the appropriate intermediates.

| Example | LCMS | ¹H NMR |
|---|---|---|
| 71 | LCMS (ESI−): | ¹H NMR (400 MHz, CD₃OD) δ 6.88-6.81 (m, 2H), 4.29-4.22 (m, 4H), |
| | m/z = 370.1 | 3.49 (br dd, J = 7.9, 11.1 Hz, 1H), 3.38-3.32 (m, 2H), 2.88 (br t, J = 10.3 |
| | (M − H)⁻ | Hz, 1H), 2.47-2.34 (m, 1H), 2.29-2.18 (m, 1H), 1.70 (qd, J = 8.5, 13.2 Hz, |
| | | 1H), 1.52-1.29 (m, 4H), 0.95 (t, J = 7.1 Hz, 3H) |
| 72 | LCMS (ESI−): | ¹H NMR (400 MHz, CD₃OD) δ 6.90-6.77 (m, 2H), 4.25 (d, J = 7.1 Hz, |
| | m/z = 384.1 | 4H), 3.55-3.43 (m, 1H), 3.34 (br s, 2H), 2.87 (br t, J = 10.1 Hz, 1H), 2.56- |
| | (M − H)⁻ | 2.39 (m, 1H), 2.31-2.15 (m, 1H), 1.77-1.50 (m, 2H), 1.44-1.30 (m, 2H), |
| | | 0.93 (dd, J = 3.8, 6.5 Hz, 6H) |
| 73 | LCMS (ESI−): | ¹H NMR (400 MHz, CD₃OD) δ 6.86-6.71 (m, 2H), 4.27 (s, 2H), 3.89 (s, |
| | m/z = 328.0 | 2H), 3.60 (t, J = 6.2 Hz, 2H), 3.14-3.09 (m, 2H), 1.79-1.73 (m, 2H), 1.64- |
| | (M − H)⁻ | 1.56 (m, 2H) |

### Example 74: Synthesis of 5-(2-fluoro-6-hydroxy-3-((1-methylpiperidin-2-yl)methyl)phenyl)-1,2,5-thiadiazolidin-3-one 1,1-dioxide

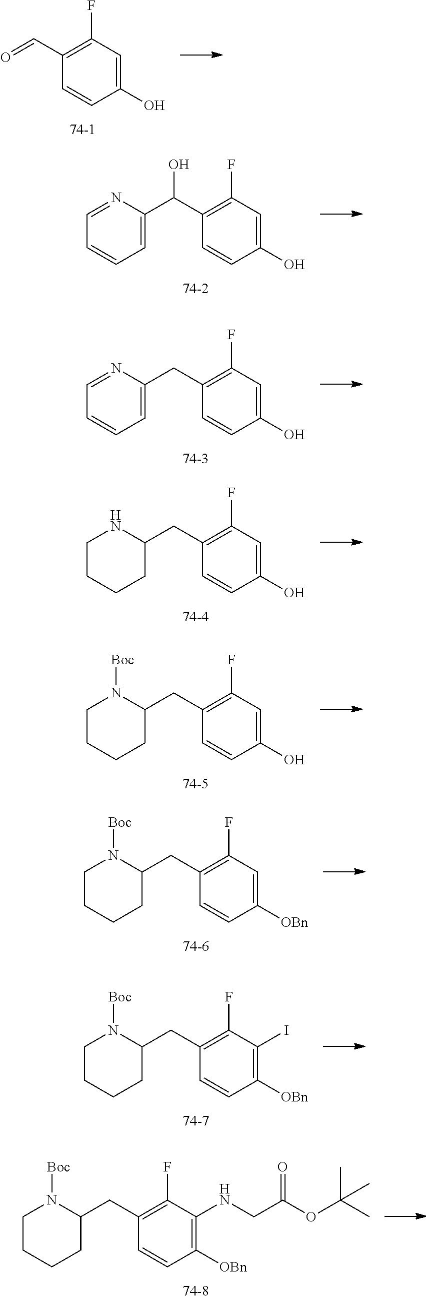

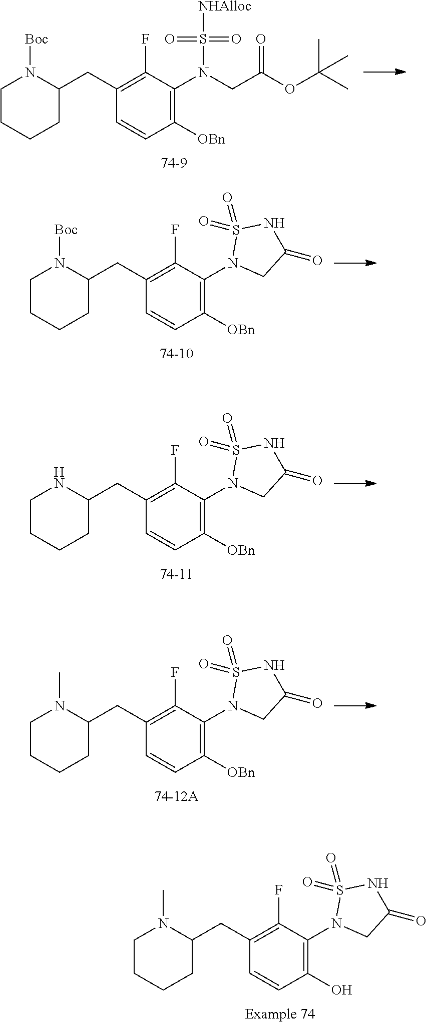

### Step 1: Synthesis of Compound 74-2

To a solution of 2-bromopyridine (5 g, 31.6 mmol, 3.01 mL, 1 eq) in THE (50 mL) was added 2.5M n-BuLi (12.6 mL, 1 eq) dropwise at −78° C. under N₂ and allowed to stir for 1 h. To this was then added a solution of 74-1 (4.43 g, 31.6 mmol, 1 eq) in THE (5 mL) and allowed to stir for an additional 0.5 h. The resulting reaction mixture was quenched with a mixture of sat. NH₄Cl_{(aq)} (80 mL) and allowed to stir for 5 minutes at 25° C. The resulting reaction mixture was then extracted with ethyl acetate (150 mL×3). The combined organic layers were washed with brine (300 mL), dried over Na₂SO₄, then filtered and concentrated under reduced pressure to give a crude. The crude was then purified by column chromatography (SiO₂, petroleum ether/ethyl acetate-4/1 to 0/1) to give 74-2 (2.56 g, 11.6 mmol, 37% yield) as a light pink solid. ¹H NMR (400 MHz, DMSO-d₆) δ 9.78 (br s, 1H), 8.49-8.35 (m, 1H), 7.78 (dt, J=1.8, 7.7 Hz, 1H), 7.56 (d, J=7.9 Hz, 1H), 7.22 (ddd, J=1.0, 4.8, 7.4 Hz, 1H), 7.13 (t, J=8.6 Hz, 1H), 6.55 (dd, J=2.3, 8.5 Hz, 1H), 6.47 (dd, J=2.3, 12.0 Hz, 1H), 5.96 (d, J=4.6 Hz, 1H), 5.83 (d, J=4.4 Hz, 1H).

### Step 2: Synthesis of Compound 74-3

To a solution of 74-2 (2.46 g, 11.22 mmol, 1 eq) in TFA (25 mL) was added Et₃SiH (6.52 g, 56.1 mmol, 9 mL, 5 eq) then heated to 60° C. and allowed to stir for 12 h. To the reaction mixture was added sat. NaHCO_{3 (aq)} (100 mL) then extracted with ethyl acetate (100 mL×3). The combined organic layers were washed with brine (300 mL), dried with Na₂SO₄, then filtered and concentrated under reduced pressure to give a crude. The crude was then purified by column chromatography (SiO₂, petroleum ether/ethyl acetate-3/1 to 2/1) to give 74-3 (1.6 g, 7.8 mmol, 70% yield) as yellow solid. ¹H NMR (400 MHz, CDCl₃) δ 8.97 (br d, J=1.4 Hz, 1H), 8.55 (d, J=4.2 Hz, 1H), 7.72 (dt, J=1.7, 7.7 Hz, 1H), 7.32 (d, J=7.7 Hz, 1H), 7.23 (dd, J=5.4, 7.0 Hz, 1H), 6.87 (t, J=8.6 Hz, 1H), 6.39-6.24 (m, 2H), 4.09 (s, 2H).

### Step 3: Synthesis of Compound 74-4

To a solution of 74-3 (1.5 g, 7.38 mmol, 1 eq) in THE (50 mL) was added 1M HCl (1.66 mL) and PtO₂ (300 mg) under N₂. The suspension was degassed under vacuum and purged with H₂ (200 psi) then allowed to stir for 8 h at 25° C. The resulting reaction mixture was filtered over celite and then concentrated under reduced pressure to give a crude. The crude was then purified by column chromatography (SiO₂, petroleum ether/ethyl acetate-1/1 to ethyl acetate/methanol-10/1) to give 74-4 (870 mg, 4.16 mmol, 56% yield) as a yellow solid. ¹H NMR (400 MHz, CD₃OD) δ 7.03 (t, J=8.6 Hz, 1H), 6.60-6.47 (m, 2H), 3.15 (br dd, J=1.9, 12.4 Hz, 1H), 2.99-2.87 (m, 1H), 2.79-2.64 (m, 3H), 1.86-1.78 (m, 1H), 1.77-1.67 (m, 2H), 1.60-1.47 (m, 1H), 1.41 (tq, J=3.3, 12.6 Hz, 1H), 1.33-1.21 (m, 1H).

### Step 4: Synthesis of Compound 74-5

To a solution of 74-4 (820 mg, 3.9 mmol, 1 eq) in THE (8.2 mL) was added triethylamine (594 mg, 5.88 mmol, 818 μL, 1.5 eq) and Boc₂O (1.28 g, 5.88 mmol, 1.35 mL, 1.5 eq) at 20° C. and allowed to stir for 1 h. To the reaction mixture was added H₂O (15 mL) then extracted with ethyl acetate (15 mL×3). The combined organic layers were washed with brine (45 mL), dried with Na₂SO₄, then filtered and concentrated under reduced pressure to give a crude. The crude was then purified by column chromatography (SiO₂, petroleum ether/ethyl acetate-7/1 to 5/1) to give 74-5 (800 mg, 2.6 mmol, 66% yield) as a yellow oil. ¹H NMR (400 MHz, CD₃OD) δ 6.98 (br t, J=8.6 Hz, 1H), 6.54-6.41 (m, 2H), 4.50-4.35 (m, 1H), 3.96 (br d, J=12.1 Hz, 1H), 3.01 (dt, J=2.1, 13.3 Hz, 1H), 2.95-2.65 (m, 2H), 1.80-1.57 (m, 5H), 1.44-1.35 (m, 1H), 1.26 (br s, 9H).

### Step 5: Synthesis of Compound 74-6

To a solution of 74-5 (800 mg, 2.6 mmol, 1 eq) in DMF (8 mL) was added K₂CO₃ (1.07 g, 7.76 mmol, 3 eq) and BnBr (884 mg, 5.17 mmol, 614 μL, 2 eq) at 20° C. and allowed to stir for 1 h. To the reaction mixture was added H₂O (15 mL) then extracted with ethyl acetate (15 mL×3). The combined organic layers were washed with brine (30 mL×5), dried with Na₂SO₄, then filtered and concentrated under reduced pressure to give a crude. The crude was then purified by column chromatography (SiO₂, petroleum ether/ethyl acetate=1/0 to 9/1) to give 74-6 (960 mg, 2.4 mmol, 94% yield) as a colorless oil. ¹H NMR (400 MHz, CDCl₃) δ 7.47-7.29 (m, 5H), 7.05 (br d, J=7.5 Hz, 1H), 6.74-6.61 (m, 2H), 5.02 (s, 2H), 4.43 (br s, 1H), 4.11-3.99 (m, 1H), 3.00-2.86 (m, 1H), 2.81 (br d, J=7.5 Hz, 2H), 1.71-1.60 (m, 3H), 1.57 (br d, J=4.9 Hz, 2H), 1.48-1.39 (m, 1H), 1.31 (s, 8H).

### Step 6: Synthesis of Compound 74-7

To a solution of 74-6 (710 mg, 1.78 mmol, 1 eq) in THE (7 mL) was added 2.5M n-BuLi (1.07 mL, 1.5 eq) at −78° C. and allowed to stir for 1 h under N₂. To this was added a solution of I₂ (676 mg, 2.67 mmol, 537 μL, 1.5 eq) in THE (7 mL) and allowed to stir for an additional 1 h. The resulting suspension was quenched with a mixture of sat. NH₄Cl_{(aq)} and 1M Na₂S₂O_{3 (aq)} (1:1, 15 mL) and allowed to stir for 5 minutes at 25° C. The resulting reaction mixture was then extracted with ethyl acetate (15 mL×3). The combined organic layers were washed with water (30 mL) and brine (30 mL), dried over Na₂SO₄, then filtered and concentrated under reduced pressure to give a crude. The crude was then purified by column chromatography (SiO₂, petroleum ether/ethyl acetate=1/0 to 8/1) to give 74-7 (650 mg, 1.24 mmol, 70% yield) as a white solid. ¹H NMR (400 MHz, CDCl₃) δ 7.52-7.46 (m, 2H), 7.40 (t, J=7.5 Hz, 2H), 7.36-7.30 (m, 1H), 7.12-6.99 (m, 1H), 6.58 (d, J=8.4 Hz, 1H), 5.14 (s, 2H), 4.45 (br s, 1H), 4.17-3.95 (m, 1H), 2.99-2.74 (m, 3H), 1.77-1.52 (m, 5H), 1.49-1.40 (m, 1H), 1.28 (br s, 9H).

### Step 7: Synthesis of Compound 74-8

A solution of 74-7 (650 mg, 1.24 mmol, 1 eq), tert-butyl 2-aminoacetate (243 mg, 1.86 mmol, 1.5 eq) and Cs₂CO₃ (1.21 g, 3.71 mmol, 3 eq) in 1,4-dioxane (6.5 mL) was degassed and purged with N₂ for 3 times. To this was then added XPhos (66 mg, 123 μmol, 0.1 eq) and BrettPhos Pd G3 (112 mg, 123 μmol, 0.1 eq) and again degassed and purged with N₂ for 3 times, then heated to 90° C. and allowed to stir for 24 h. To the reaction mixture was added H₂O (15 mL) then extracted with ethyl acetate (15 mL×3). The combined organic layers were washed with brine (50 mL), dried with Na₂SO₄, then filtered and concentrated under reduced pressure to give a crude. The crude was then purified by column chromatography (SiO₂, petroleum ether/ethyl acetate=1/0 to 7/1) to give 74-8 (560 mg, 1.06 mmol, 85% yield) as a yellow oil. ¹H NMR (400 MHz, CDCl₃) δ 7.46-7.30 (m, 5H), 6.62-6.56 (m, 1H), 6.53 (br d, J=5.9 Hz, 1H), 5.08 (s, 2H), 4.41 (br s, 1H), 4.00 (t, J=2.1 Hz, 2H), 2.97-2.70 (m, 3H), 1.69-1.51 (m, 6H), 1.45 (s, 9H), 1.35 (s, 9H).

### Step 8: Synthesis of Compound 74-9

To a solution of N-(oxomethylene)sulfamoyl chloride (225 mg, 1.6 mmol, 138 μL, 1.5 eq) in CH₂Cl₂ (6 mL) was added prop-2-en-1-ol (100 mg, 1.7 mmol, 117 μL, 1.63 eq) at 0° C. under N₂ and allowed to stir for 0.5 h. To this was then added a solution of 74-8 (560 mg, 1.06 mmol, 1 eq) and triethylamine (214 mg, 2.1 mmol, 295 μL, 2 eq) in CH₂Cl₂ (6 mL) and allowed to stir for an additional 0.5 h. To the reaction mixture was added H₂O (20 mL) then extracted with CH₂Cl₂ (15 mL×3). The combined organic layers were washed with brine (40 mL), dried with Na₂SO₄, then filtered and concentrated under reduced pressure to give 74-9 (786 mg, crude) as yellow oil. ¹H NMR (400 MHz, CDCl₃) δ 7.50-7.30 (m, 5H), 7.16-7.01 (m, 1H), 6.75-6.62 (m, 1H), 5.97-5.79 (m, 1H), 5.43-5.23 (m, 3H), 5.15-4.96 (m, 2H), 4.57-4.51 (m, 2H), 3.20 (q, J=7.4 Hz, 1H), 2.96-2.76 (m, 3H), 1.69-1.51 (m, 6H), 1.41 (d, J=8.4 Hz, 9H), 1.33-1.24 (m, 9H).

### Step 9: Synthesis of Compound 74-10

To a solution of 74-9 (776 mg, 1.12 mmol, 1 eq) in MeOH (8 mL) was added NaOMe (606 mg, 3.37 mmol, 3 eq) and Pd(PPh₃)₄ (26 mg, 22 μmol, 0.02 eq) under N₂ then heated to 60° C. for 0.5 h. To the reaction mixture was added 1M HCl (15 mL) then extracted with ethyl acetate (15 mL×3). The combined organic layers were washed with brine (45 mL), dried with Na₂SO₄, then filtered and concentrated under reduced pressure to give 74-10 (540 mg, crude) as yellow oil. ¹H NMR (400 MHz, CDCl₃) δ 7.50-7.46 (m, 1H), 7.43-7.39 (m, 2H), 7.39-7.32 (m, 2H), 7.25-7.00 (m, 1H), 6.76 (dd, J=1.3, 8.7 Hz, 1H), 5.16-5.09 (m, 2H), 4.47-4.36 (m, 2H), 2.98-2.73 (m, 3H), 1.75-1.48 (m, 6H), 1.34-1.26 (m, 9H).

### Step 10: Synthesis of Compound 74-11

To 74-10 (540 mg, 1.01 mmol, 1 eq) was added 4M HCUethyl acetate (10 mL) at 20° C. and allowed to stir for 0.5 h. The reaction mixture was then concentrated under reduced pressure to give 1-11 (472 mg, crude) as a yellow solid. ¹H NMR (400 MHz, CD₃OD) δ 7.59-7.51 (m, 1H), 7.48 (br d, J 6.9 Hz, 1H), 7.42-7.33 (m, 3H), 7.32-7.28 (m, 1H), 7.06-6.97 (m, 1H), 5.25-5.15 (m, 2H), 4.49-4.38 (m, 2H), 3.41-3.33 (m, 2H), 3.00-2.86 (m, 3H), 1.95-1.86 (m, 3H), 1.66 (br dd, J=3.3, 11.7 Hz, 1H), 1.58-1.47 (m, 2H).

### Step 11: Synthesis of Compound 74-12A

To a solution of 74-11 (200 mg, 425 μmol, 1 eq), formaldehyde (107 mg, 1.3 mmol, 98 μL, 3.1 eq) in acetonitrile (2 mL) was added acetic acid (64 mg, 1.06 mmol, 61 μL, 2.5 eq) at 20° C. and allowed to stir for 0.5 h. To this was then added NaBH₃CN (29 mg, 468 μmol, 1.1 eq) and allowed to stir for an additional 1 h. The reaction mixture was then filtered and concentrated under reduced pressure to give 74-12A (190 mg, crude) as colorless oil. LCMS (ESI−): m/z=446.1 (M−H)⁻.

### Step 12: Synthesis of Compound Example 74

To a solution of 74-12A (170 mg, 380 μmol, 1 eq) in MeOH (15 mL) was added 10% Pd/C (170 mg) under N₂. The suspension was degassed under vacuum and purged with H₂ (15 psi) 3 times then allowed to stir for 12 h at 25° C. The resulting reaction mixture was filtered over celite and then concentrated under reduced pressure to give a crude. The crude was then purified by HPLC to give Example 74 (22 mg, 62 μmol, 16% yield). LCMS (ESI−): m/z=356.1 (M−H)⁻. ¹H NMR (400 MHz, D₂O) δ 7.12 (t, J=8.8 Hz, 1H), 6.69 (d, J=8.8 Hz, 1H), 4.35 (s, 2H), 3.50-3.00 (m, 5H), 2.92 (br s, 3H), 2.81-2.67 (m, 1H), 1.86-1.73 (m, 3H), 1.54-1.35 (m, 2H).

Examples 75-80 were prepared according to the procedures described in Example 74 using the appropriate intermediates.

| Example | LCMS | ¹H NMR |
|---|---|---|
| 75 | LCMS (ESI−): | ¹H NMR (400 MHz, D₂O) δ 7.18 (t, J = 8.3 Hz, 1H), 6.79 (d, J = 8.5 Hz, |
| | m/z = 342.1 | 1H), 4.37 (s, 2H), 3.40-3.25 (m, 2H), 2.97-2.85 (m, 3H), 1.94 (br d, J = 8.2 |
| | (M − H)⁻ | Hz, 1H), 1.88-1.80 (m, 2H), 1.66-1.53 (m, 1H), 1.50-1.38 (m, 2H) |
| 76 | LCMS (ESI−): | ¹H NMR (400 MHz, CD₃OD) δ 7.13 (t, J = 8.3 Hz, 1H), 6.74 (d, J = 8.7 |
| | m/z = 412.2 | Hz, 1H), 4.27 (s, 2H), 3.55-3.40 (m, 2H), 3.29-3.03 (m, 3H), 3.01-2.67 (m, |
| | (M − H)⁻ | 1H), 1.96-1.51 (m, 9H), 1.00 (dd, J = 2.9, 6.2 Hz, 6H), 0.95 (dd, J = 6.4, |
| | | 9.2 Hz, 1H) |
| 77 | LCMS (ESI−): | ¹H NMR (400 MHz, CD₃OD) δ 7.14 (t, J = 8.5 Hz, 1H), 6.74 (dd, J = 1.3, |
| | m/z = 438.0 | 8.5 Hz, 1H), 4.27 (s, 2H), 3.74-3.44 (m, 4H), 3.13 (br s, 2H), 2.83 (br s, |
| | (M − H)⁻ | 3H), 1.86 (br s, 4H), 1.54 (br s, 2H) |
| 78 | LCMS (ESI−): | ¹H NMR (400 MHz, CD₃OD) δ 7.24-7.52 (m, 5H) 7.05 (s, 1H) 6.69 (d, J = |
| | m/z = 446.1 | 3.58 Hz, 1H) 4.26 (s, 2H) 3.43-3.74 (m, 4H) 3.21-3.29 (m, 1H) 2.95-3.20 |
| | (M − H)⁻ | (m, 3H) 2.74-2.94 (m, 1H) 1.74-2.06 (m, 4H) 1.42-1.71 (m, 2H) |
| 79 | LCMS (ESI−): | ¹H NMR (400 MHz, CD₃OD) δ 7.13 (t, J = 8.4 Hz, 1H), 6.74 (dd, J = 0.9, |
| | m/z = 426.1 | 8.5 Hz, 1H), 4.26 (s, 2H), 3.57-3.39 (m, 2H), 3.27-3.10 (m, 3H), 2.94-2.67 |
| | (M − H)⁻ | (m, 1H), 1.93-1.70 (m, 6H), 1.64 (tt, J = 6.7, 13.4 Hz, 2H), 1.58-1.46 (m, |
| | | 1H), 1.28 (br d, J = 7.9 Hz, 2H), 0.95 (d, J = 6.6 Hz, 6H) |
| 80 | LCMS (ESI−): | ¹H NMR (400 MHz, CD₃OD) δ 7.13 (t, J = 8.5 Hz, 1H), 6.74 (dd, J = 1.4, |
| | m/z = 438.1 | 8.5 Hz, 1H), 4.27 (s, 2H), 3.58-3.38 (m, 2H), 3.26-3.06 (m, 3H), 2.94-2.70 |
| | (M − H)⁻ | (m, 1H), 1.95-1.49 (m, 15H), 1.38-1.11 (m, 3H) |
| 81 | LCMS (ESI−): | ¹H NMR (400 MHz, CD₃OD) δ 7.15 (t, J = 8.3 Hz, 1H), 6.75 (d, J = 8.7 |
| | m/z = 412.2 | Hz, 1H), 4.26 (s, 2H), 3.48-3.39 (m, 2H), 3.26-3.22 (m, 3H), 2.80-2.67 (m, |
| | (M − H)⁻ | 1H), 1.86-1.63 (m, 10H), 1.01 (dd, J = 2.9, 6.2 Hz, 6H) |
| 82 | LCMS (ESI−): | ¹H NMR (400 MHz, CD₃OD) δ 7.15 (t, J = 8.3 Hz, 1H), 6.75 (d, J = 8.7 |
| | m/z = 412.2 | Hz, 1H), 4.26 (s, 2H), 3.48-3.39 (m, 2H), 3.26-3.13 (m, 2H), 2.80-2.67 (m, |
| | (M − H)⁻ | 2H), 1.85-1.61 (m, 10H), 0.99 (dd, J = 2.9, 6.2 Hz, 6H) |

### Example 83: Synthesis of 5-(2-fluoro-6-hydroxy-4-(2-(pyrrolidin-1-yl)ethyl)phenyl)-1,2,5-thiadiazolidin-3-one 1,1-dioxide

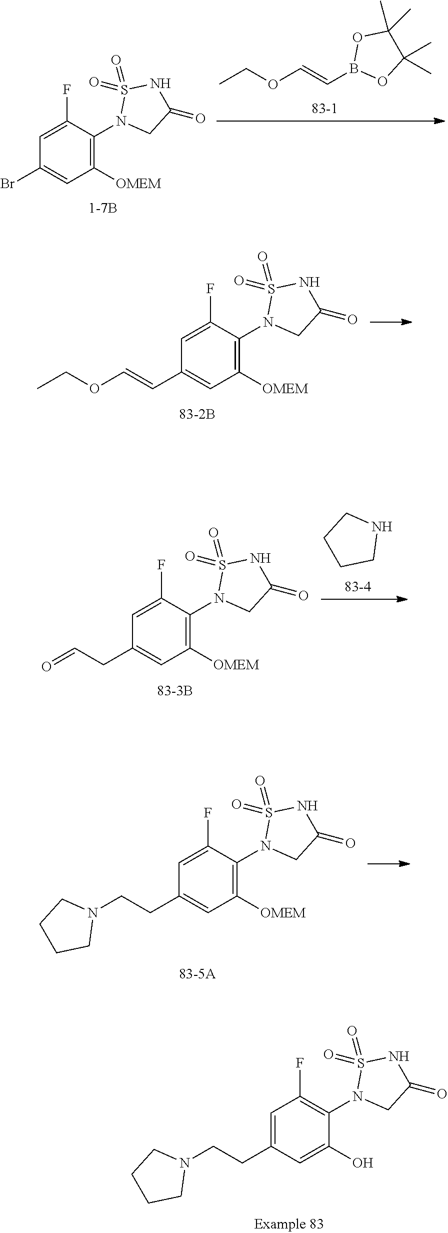

### Step 1: Synthesis of Compound 83-2B

To a solution of 1-7B (4.4 g, 10.65 mmol, 1 eq) in DME (44 mL) and H₂O (4.4 mL) was added Pd(dppf)Cl₂ (779 mg, 1.06 mmol, 0.1 eq), K₂CO₃ (4.41 g, 31.94 mmol, 3 eq) and 83-1 (4.22 g, 21.30 mmol, 2 eq) then heated to 95° C. and allowed to stir for 2 h. To the resulting reaction mixture was then added to H₂O (50 mL) then extracted with ethyl acetate (50 mL×3). The combined organic layers were washed with brine (50 mL), dried with Na₂SO₄, then filtered and concentrated under reduced pressure to give a crude. The crude was then purified by column chromatography on silica gel eluting ethyl acetate/methanol-99/1 to 80/20 to give 83-2B (5 g, crude yield). ¹H NMR (CDCl₃) δ 6.95 (br d, J=12.84 Hz, 1H) 6.78 (br s, 1H) 6.58 (br d, J=10.51 Hz, 1H) 5.65 (br d, J=12.59 Hz, 1H) 5.09-5.37 (m, 2H) 4.21 (br s, 2H) 3.85 (q, J=6.77 Hz, 2H) 3.75 (br s, 2H) 3.44 (br s, 2H) 3.26 (s, 3H) 1.31 (br t, J=6.97 Hz, 3H).

### Step 2: Synthesis of Compound 83-3B

To a solution of 83-2B (200 mg, 494 μmol, 1 eq) in CH₂Cl₂ (4 mL) was added TFA (308 mg, 2.70 mmol, 200 μL, 5.4 eq) and allowed to stir for 0.5 h at 0° C. The resulting reaction mixture was then neutralized with diethylamine to reach a pH-8-9. This was then concentrated under reduced pressure to give 83-3B. LCMS (ESI−): m/z=375.2 (M−H)⁻.

### Step 3: Synthesis of Compound 83-5A

To a solution of 83-3B (500 mg, 1.3 mmol, 1 eq) in CH₂Cl₂ (2 mL) was added 83-4 (188 mg, 2.66 mmol, 220 μL, 2 eq) and diisopropylethylamine (515 mg, 4 mmol, 694 μL, 3 eq) and allowed to stir for 0.5 h at 25° C. To this was then added NaBH₃CN (250 mg, 4 mmol, 3 eq) and allowed to stir for an additional 0.5 h. To the resulting reaction mixture was then added to H₂O (10 mL) then extracted with CH₂Cl₂ (10 mL×3). The combined organic layers were washed with brine (10 mL), dried with Na₂SO₄, then filtered and concentrated under reduced pressure to give a crude. The crude was then purified by HPLC to give 83-5A (210 mg, 36.6% yield) as a yellow oil. LCMS (ESI+): m/z=432.2 (M+H)⁺.

### Step 4: Synthesis of Compound Example 83

To a solution of 83-5A (0.02 g, 46.14 μmol, 1 eq) in THE (1 mL) was added 4M HCl/dioxane (1 mL) and allowed to stir for 1 h at 0° C. The reaction mixture was then concentrated under reduced pressure to give a crude. The crude was then purified by HPLC to give Example 83 (2.16 mg, 100% purity). LCMS (ESI−): m/z=342.2 (M−H)⁻. ¹H NMR (400 MHz, D₂O) (36.73 (m, 2H), 4.35 (s, 2H), 3.46 (m, 3H), 3.31 (m, 3H), 2.98 (m, 2H), 2.02 (s, 4H).

### Example 84: Synthesis of 5-(4-(2-(2-ethylpyrrolidin-1-yl)ethyl)-2-fluoro-6-hydroxyphenyl)-1,2,5-thiadiazolidin-3-one 1,1-dioxide

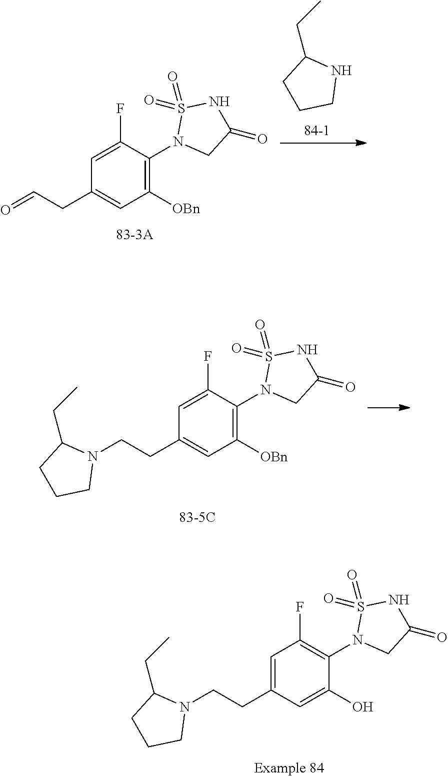

Compound 83-5C was prepared according to the procedure described for 83-5A. To a solution of 83-5C (0.02 g, 46.14 μmol, 1 eq) in MeOH (1 mL) was added Pd/C (0.02 g, 10% purity) under N₂. The suspension was degassed under vacuum and purged with H₂ (15 psi) 3 times. This was then allowed to stir for 1 h at 40° C. The resulting reaction mixture was filtered over celite and the filtrate was concentrated under reduced pressure to give a crude. The crude was then purified by HPLC to give Example 84 (2 mg). LCMS (ESI+): m/z=372.2 (M+H)⁺. ¹H NMR (400 MHz, D₂O) δ 6.75 (m, 2H), 4.35 (s, 2H), 3.63 (m, 2H), 3.27-3.15 (m, 3H), 3.04-2.98 (m, 2H), 2.31 (m, 1H), 2.07-1.96 (m, 3H), 1.73 (m, 1H), 1.52 (m, 1H), 0.96-0.92 (t, 3H).

Example 85-128 were prepared according to the procedures described in Example 83 using the appropriate intermediates.

| Example | LCMS | ¹H NMR |
|---|---|---|
| 85 | LCMS (ESI+): | ¹H NMR (400 MHz, D₂O) δ 6.73 (m, 2H), 4.35 (s, 2H), 3.45 (m, 4H), |
| | m/z = 400.2 | 2.98 (m, 3H), 2.43 (m, 1H), 2.20 (m, 1H), 1.64 (m, 1H), 1.53(m, 1 H), |
| | (M + H)⁺ | 1.32 (m, 1H), 0.85 (m, 6H) |
| 86 | LCMS (ESI+): | ¹H NMR (400 MHz, D₂O) δ 6.73 (m, 2H), 4.35 (s, 2H), 3.45 (m, 5H), |
| | m/z = 386.2 | 3.00 (m, 2H), 2.87 (m, 1H), 2.37 (m, 1H), 2.19 (m, 1H), 1.67(m, 1 H), |
| | (M + H)⁺ | 1.40-1.37 (m, 2H), 131-1.27 (m, 2H), 0.85 (t, 3H) |
| 87 | LCMS (ESI+): | ¹H NMR (400 MHz, D₂O) δ 6.62 (m, 2H), 4.29 (s, 2H), 3.39 (m, 1H), |
| | m/z = 372.2 | 3.34 (m, 4H), 2.93 (m, 2H), 2.23 (m, 2H), 1.61 (m, 1H), 1.38 (m, 3H), |
| | (M + H)⁺ | 0.82 (t, 3H) |
| 88 | LCMS (ESI+): | ¹H NMR (400 MHz, D₂O) δ 6.52 (m, 2H), 4.33 (s, 2H), 3.48 (m, 4H), |
| | m/z = 414.2 | 2.94 (m, 2H), 2.31 (m, 1H), 2.20 (m, 1H), 1.73 (m, 1H), 1.51 (m, 1H), |
| | (M + H)⁺ | 1.34 (m, 2H), 1.18 (m, 3H), 0.82 (m, 7H) |
| 89 | LCMS (ESI−): | ¹H NMR (400 MHz, D₂O) δ 6.70 (m, 2H), 4.33 (s, 2H), 3.99 (s, 3H), |
| | m/z = 358.0 | 3.54 (m, 1H), 3.33 (m, 5H), 3.00 (m, 2H), 1.61 (m, 1H) |
| | (M − H)⁻ | |
| 90 | LCMS (ESI−): | ¹H NMR (400 MHz, D₂O) δ 6.70 (m, 2H), 4.33 (s, 2H), 3.52 (s, 2H), |
| | m/z = 356.1 | 3.32 (m, 2H), 3.00 (m, 4H), 1.93-1.40 (m, 6H) |
| | (M − H)⁻ | |
| 91 | LCMS (ESI−): | ¹H NMR (400 MHz, D₂O) δ 6.71 (m, 2H), 4.34 (s, 2H), 3.09 (s, 2H), |
| | m/z = 378.0 | 2.91 (m, 2H), 2.88 (m, 4H), 2.33 (m, 2H) |
| | (M − H)⁻ | |
| 92 | LCMS (ESI−): | ¹H NMR (400 MHz, D₂O) δ 9.44 (s, 1H), 8.96 (m, 1H), 6.64-6.60 (m, |
| | m/z = 360.1 | 2H), 3.93 (s, 2H), 3.40 (m, 2H), 3.29 (m, 2H), 3.10 (m, 2H), 3.03 (m, |
| | (M − H)⁻ | 2H), 1.54 (m, 4H), 1.04 (m, 6H) |
| 93 | LCMS (ESI−): | ¹H NMR (400 MHz, DMSO-d₆) δ 6.75 (m, 2H), 5.50 (d, 1H), 4.35 (s, |
| | m/z = 384.1 | 2H), 3.77 (m, 2H), 3.55 (m, 4H), 3.04 (m, 2H), 2.35 (m, 1H), 2.32 (m, |
| | (M − H)⁻ | 1H) |
| 94 | LCMS (ESI−): | ¹H NMR (400 MHz, D₂O) δ 6.79-6.65 (m, 2H), 5.57-5.31 (m, 1H), 4.34 |
| | m/z = 360.0 | (s, 2H), 3.78-3.55 (m, 2H), 3.52-3.29 (m, 4H), 2.99 (t, J = 7.6 Hz, 2H), |
| | (M − H)⁻ | 2.47-2.24 (m, 2H) |
| 95 | LCMS (ESI−): | ¹H NMR (400 MHz, D₂O) δ 6.78-6.66 (m, 2H), 5.53-5.34 (m, 1H), 4.34 |
| | m/z = 360.0 | (s, 2H), 3.81-3.57 (m, 2H), 3.52-3.32 (m, 4H), 3.06-2.93 (m, 2H), 2.48- |
| | (M − H)⁻ | 2.25 (m, 2H) |
| 96 | LCMS (ESI−): | ¹H NMR (400 MHz, D₂O) δ 6.76 (br s, 2H), 4.37 (s, 2H), 3.65 (br d, |
| | m/z = 410.1 | J = 8.6 Hz, 1H), 3.60-3.31 (m, 6H), 3.02 (br t, J = 7.5 Hz, 2H), 2.52-2.14 |
| | (M − H)⁻ | (m, 2H) |
| 97 | LCMS (ESI−): | ¹H NMR (400 MHz, D₂O) δ 6.86-6.59 (m, 2H), 5.22-4.99 (m, 1H), 4.34 |
| | m/z = 374.0 | (s, 2H), 3.80-3.61 (m, 1H), 3.52 (br d, J = 12.4 Hz, 1H), 3.40-3.19 (m, |
| | (M − H)⁻ | 3H), 3.13-2.96 (m, 3H), 2.19-1.97 (m, 2H), 1.93-1.63 (m, 2H) |
| 98 | LCMS (ESI−): | ¹H NMR (400 MHz, D₂O) δ = 6.76 (t, J = 4.7 Hz, 2H), 4.37 (s, 2H), |
| | m/z = 392.0 | 3.49-3.33 (m, 6H), 3.07-2.99 (m, 2H), 2.42-2.25 (m, 4H) |
| | (M − H)⁻ | |
| 99 | LCMS (ESI−): | ¹H NMR (400 MHz, CD₃OD) δ 6.74-6.67 (m, 2H), 4.40 (s, 2H), 3.72- |
| | m/z = 382.0 | 3.61 (m, 2H), 3.42 (br t, J = 8.0 Hz, 2H), 3.26-3.19 (m, 1H), 3.15 (d, |
| | (M − H)⁻ | J = 11.8 Hz, 1H), 3.03-2.91 (m, 2H), 2.31-2.08 (m, 5H), 2.07-1.86 (m, 3H) |
| 100 | LCMS (ESI+): | ¹H NMR (400 MHz, D₂O) δ = 3.00-3.09 (m, 2 H) 3.47-3.55 (m, 2 H) |
| | m/z = 359.3 | 3.55-3.69 (m, δ H) 4.34 (s, 2 H) 6.69-6.78 (m, 2 H) |
| | (M + H)⁺ | |
| 101 | LCMS (ESI−): | ¹H NMR (400 MHz, D₂O) δ 6.71-6.61 (m, 2H), 4.39-4.27 (m, 2H), 4.05- |
| | m/z = 372.1 | 3.87 (m, 1H), 3.48-3.33 (m, 2H), 3.32-3.22 (m, 2H), 3.19-3.03 (m, 2H), |
| | (M − H)⁻ | 2.95 (br t, J = 7.4 Hz, 2H), 2.13-1.99 (m, 2H), 1.85-1.65 (m, 2H) |
| 102 | LCMS (ESI−): | ¹H NMR (400 MHz, D₂O) δ 6.64-6.68 (m, 2 H) 4.30 (s, 2 H) 3.48-3.60 |
| | m/z = 372.0 | (m, 3 H) 3.42 (t, J = 7.57 Hz, 2 H) 3.36 (br s, 2 H) 3.04-3.17 (m, 1 H) |
| | (M − H)⁻ | 2.95 (t, J = 7.63 Hz, 2 H) 2.59 (dt, J = 14.23, 6.96 Hz, 1 H) 2.14 (br dd, |
| | | J = 14.20, 7.32 Hz, 1 H) 1.69-1.82 (m, 1 H) |
| 103 | LCMS (ESI−): | ¹H NMR (400 MHz, D₂O) δ 6.67-6.79 (m, 2 H), 4.61 (tt, J = 5.93, 2.83 |
| | m/z = 397.1 | Hz, 1 H), 4.34 (s, 2 H), 3.42 (dd, J = 12.82, 5.69 Hz, 1 H), 3.19-3.36 (m, |
| | (M − H)⁻ | 4 H), 2.95-3.08 (m, 3 H), 2.41 (td, J = 14.85, 8.69 Hz, 1 H), 1.95 (ddd, |
| | | J = 10.51, 7.00, 3.38 Hz, 1 H) |
| 104 | LCMS (ESI−): | ¹H NMR (400 MHz, D₂O) δ 6.66-6.73 (m, 2 H) 4.32 (s, 2 H) 3.63 (br d, |
| | m/z = 386.1 | J = 1.34 Hz, 1 H) 3.35 (br s, 1 H) 3.32 (s, 5 H) 3.30 (s, 1 H) 3.11-3.26 (m, |
| | (M − H)⁻ | 2 H) 2.95-3.01 (m, 3 H) 2.00-2.17 (m, 2 H) 1.78-1.90 (m, 1 H) |
| 105 | LCMS (ESI−): | ¹H NMR (400 MHz, DMSO-d₆) δ 9.43 (s, 1H), 9.28-8.79 (m, 1H), 6.68- |
| | m/z = 396.1 | 6.55 (m, 2H), 3.93 (s, 2H), 3.38 (br s, 2H), 3.27-3.20 (m, 2H), 2.99-2.79 |
| | (M − H)⁻ | (m, 4H), 1.92-1.73 (m, 8H), 1.70-1.54 (m, 2H) |
| 106 | LCMS (ESI−): | ¹H NMR (400 MHz, D₂O) δ 6.75 (s, 2H), 4.36 (s, 2H), 3.99-3.75 (m, |
| | m/z = 374.1 | 2H), 3.64-3.20 (m, 4H), 3.11-2.90 (m, 2H), 2.61-2.05 (m, 2H), 1.67- |
| | (M − H)⁻ | 1.51 (m, 3H). |
| 107 | LCMS (ESI−): | ¹H NMR (400 MHz, D₂O) δ 6.76 (br t, J = 4.4 Hz, 2H), 4.36 (s, 2H), |
| | m/z = 382.1 | 3.56 (br d, J = 12.1 Hz, 2H), 3.47-3.34 (m, 2H), 3.13 (br t, J = 11.9 Hz, |
| | (M − H)⁻ | 2H), 3.08-3.01 (m, 2H), 2.12 (br t, J = 12.3 Hz, 2H), 1.22 (br d, J = 14.4 |
| | | Hz, 2H), 0.54-0.35 (m, 4H). |
| 108 | LCMS (ESI−): | ¹H NMR (400 MHz, D₂O) δ 6.76 (br s, 2H), 4.38 (s, 2H), 3.80-3.67 (m, |
| | m/z = 370.1 | 1H), 3.57-3.39 (m, 3H), 3.38-3.22 (m, 1H), 3.09-2.87 (m, 3H), 2.10- |
| | (M − H)⁻ | 1.78 (m, 2H), 1.29-1.08 (m, 6H). |
| 109 | LCMS (ESI−): | ¹H NMR (400 MHz, D₂O) δ 6.71-6.80 (m, 2 H), 4.35 (s, 2 H), 3.54-3.79 |
| | m/z = 370.1 | (m, 2 H), 3.45 (q, J = 7.07 Hz, 2 H), 3.04-3.35 (m, 2 H), 2.99 (t, J = 7.65 |
| | (M − H)⁻ | Hz, 2 H), 2.33-2.76 (m, 1 H), 2.11-2.25 (m, 1 H), 1.52-1.79 (m, 1 H), |
| | | 1.31-1.51 (m, 2 H), 0.87 (td, J = 7.47, 3.51 Hz, 3 H) |
| 110 | LCMS (ESI−): | ¹H NMR (400 MHz, D₂O) δ 6.71-6.80 (m, 2 H), 4.35 (s, 2 H), 3.54-3.79 |
| | m/z = 370.1 | (m, 2 H), 3.45 (q, J = 7.07 Hz, 2 H), 3.04-3.35 (m, 2 H), 2.99 (t, J = 7.65 |
| | (M − H)⁻ | Hz, 2 H), 2.33-2.76 (m, 1 H), 2.11-2.25 (m, 1 H), 1.52-1.79 (m, 1 H), |
| | | 1.31-1.51 (m, 2 H), 0.87 (td, J = 7.47, 3.51 Hz, 3 H) |
| 111 | LCMS (ESI+): | ¹H NMR (400 MHz, Acetonitrile-d₃) δ 6.76 (s, 1 H) 6.70 (m, 1 H) 4.13 |
| | m/z = 400.3 | (s, 2 H) 3.36 (br t, J = 6.75 Hz, 2 H) 2.96 (t, J = 7.44 Hz, 2 H) 2.12-2.13 |
| | (M + H)⁺ | (m, 2 H) 1.99 (br s, 1 H) 1.80 (m, 2 H) 1.31 (br s, 2 H) 0.94 (s, 9 H) |
| 112 | LCMS (ESI−): | ¹H NMR (400 MHz, D₂O) δ 6.79-6.67 (m, 2H), 4.39-4.33 (m, 2H), 3.50- |
| | m/z = 396.1 | 3.30 (m, 4H), 3.28-3.16 (m, 1H), 3.04-2.93 (m, 2H), 2.01-1.88 (m, 2H), |
| | (M − H)⁻ | 1.63 (br s, 7H) |
| 113 | LCMS (ESI+): | ¹H NMR (400 MHz, DMSO-d₆) δ 9.56-9.75 (m, 1 H) 9.43 (br s, 1 H) |
| | m/z = 414.3 | 6.65 (br d, J = 11.13 Hz, 1 H) 6.62 (s, 1 H) 3.94 (s, 2 H) 3.56 (br s, 6 H) |
| | (M + H)⁺ | 3.37 (br s, 2 H) 3.24 (m, 1 H) 2.92-3.06 (m, 1 H) 2.86 (br t, J = 7.19 Hz, |
| | | 2 H) 1.74-2.04 (m, 2 H) 1.55 (br s, 4 H) |
| 114 | LCMS (ESI+): | ¹H NMR (400 MHz, D₂O) δ 6.66-6.75 (m, 2 H), 4.34 (s, 2 H), 3.95-4.02 |
| | m/z = 384.3 | (m, 2 H), 3.83-3.90 (m, 2 H), 3.45 (t, J = 7.03 Hz, 2 H), 2.85 (t, J = 7.09 |
| | (M + H)⁺ | Hz, 2 H), 1.78-1.85 (m, 2 H), 1.75 (t, J = 7.09 Hz, 2 H), 1.44-1.59 (m, 4 H) |
| 115 | LCMS (ESI+): | ¹H NMR (400 MHz, D₂O) δ 6.66-6.80 (m, 2 H), 4.34 (s, 2 H), 3.85-3.95 |
| | m/z = 400.3 | (m, 2 H), 3.61-3.80 (m, 4 H), 3.42-3.57 (m, 2 H), 3.13-3.34 (m, 2 H), |
| | (M + H)⁺ | 2.94-3.05 (m, 2 H), 2.14-2.26 (m, 1 H), 1.95-2.12 (m, 3 H) |
| 116 | LCMS (ESI+): | ¹H NMR (400 MHz, D₂O) δ 6.67-6.79 (m, 2 H), 4.35 (s, 2 H), 3.09-3.81 |
| | m/z = 412.3 | (m, 6 H), 2.99 (br t, J = 7.69 Hz, 2 H), 1.91 (br d, J = 2.25 Hz, 2 H), |
| | (M + H)⁺ | 1.28-1.61 (m, 10 H) |
| 117 | LCMS (ESI−): | ¹H NMR (400 MHz, D₂O) δ 6.76 (br s, 2H), 4.59-4.13 (m, 2H), 3.89- |
| | m/z = 386.0 | 3.61 (m, 2H), 3.57-3.40 (m, 4H), 3.39-3.29 (m, 3H), 3.25-3.09 (m, 1H), |
| | (M − H)⁻ | 3.01 (br t, J = 7.4 Hz, 2H), 2.93-2.54 (m, 2H), 2.34-2.08 (m, 1H), 1.94- |
| | | 1.65 (m, 1H) |
| 118 | LCMS (ESI−): | ¹H NMR (400 MHz, CD₃OD) δ 6.62-6.74 (m, 2 H) 4.26 (s, 2 H) 3.63- |
| | m/z = 412.1 | 3.71 (m, 2 H) 3.25-3.29 (m, 1 H) 2.93-3.01 (m, 4 H) 2.01 (br d, J = |
| | (M − H)⁻ | 14.25 Hz, 2 H) 1.49-1.63 (m, 2 H) 1.26-1.45 (m, 2 H) 0.93 (s, 9 H) |
| 119 | LCMS (ESI+): | ¹H NMR (400 MHz, D₂O) δ 6.66-6.78 (m, 2 H), 4.32 (s, 2 H), 3.48-3.71 |
| | m/z = 400.3 | (m, 2 H), 3.36-3.47 (m, 2 H), 3.00-3.31 (m, 2 H), 2.96 (t, J = 7.38 Hz, 2 |
| | (M + H)⁺ | H), 2.06-2.59 (m, 2 H), 1.43-1.79 (m, 2 H), 1.22-1.33 (m, 2 H), 0.81 (td, |
| | | J = 4.31, 2.00 Hz, 6 H) |
| 120 | LCMS (ESI+): | ¹H NMR (400 MHz, D₂O) δ 6.66-6.78 (m, 2 H), 4.32 (s, 2 H), 3.48-3.71 |
| | m/z = 400.2 | (m, 2 H), 3.36-3.47 (m, 2 H), 3.00-3.31 (m, 2 H), 2.96 (t, J = 7.38 Hz, 2 |
| | (M + H)⁺ | H), 2.06-2.59 (m, 2 H), 1.43-1.79 (m, 2 H), 1.22-1.33 (m, 2 H), 0.81 (td, |
| | | J = 4.31, 2.00 Hz, 6 H) |
| 121 | LCMS (ESI−): | ¹H NMR (400 MHz, CD₃OD) δ 6.69-6.60 (m, 2H), 4.23 (s, 2H), 3.56 (br |
| | m/z = 410.1 | s, 2H), 3.27 (br s, 2H), 3.02-2.84 (m, 4H), 2.18-2.01 (m, 3H), 1.98-1.87 |
| | (M − H)⁻ | (m, 3H), 1.86-1.71 (m, 3H), 1.60-1.44 (m, 1H), 1.31 (br d, J = 12.8 Hz, 2H) |
| 122 | LCMS (ESI+): | ¹H NMR (400 MHz, D₂O) δ 6.71-6.79 (m, 2 H), 4.34 (s, 2 H), 3.67-4.02 |
| | m/z = 412.1 | (m, 2 H), 3.43-3.63 (m, 3 H), 3.13-3.40 (m, 2 H), 3.02 (br t, J = 7.57 Hz, |
| | (M + H)⁺ | 2 H), 2.07-2.56 (m, 2 H) |
| 123 | LCMS (ESI+): | ¹H NMR (400 MHz, D₂O) δ 6.71-6.79 (m, 2 H), 4.34 (s, 2 H), 3.67-4.02 |
| | m/z = 412.1 | (m, 2 H), 3.43-3.63 (m, 3 H), 3.13-3.40 (m, 2 H), 3.02 (br t, J = 7.57 Hz, |
| | (M + H)⁺ | 2 H), 2.07-2.56 (m, 2 H) |
| 124 | LCMS (ESI−): | ¹H NMR (400 MHz, CD₃OD) δ 6.54-6.70 (m, 2 H) 4.24 (s, 2 H) 4.10 (br |
| | m/z = 368.1 | s, 4 H) 3.38 (t, J = 7.40 Hz, 2 H) 2.79 (t, J = 7.40 Hz, 2 H) 2.28 (br t, |
| | (M − H)− | J = 7.40 Hz, 4 H) 1.87 (quin, J = 7.73 Hz, 2 H) |
| 125 | LCMS (ESI−): | ¹H NMR (400 MHz, DMSO-d₆) δ 10.14 (br s, 1H), 9.73 (br d, J = 2.0 |
| | m/z = 384.1 | Hz, 1H), 6.72-6.62 (m, 2H), 4.21 (s, 2H), 3.52 (br d, J = 11.3 Hz, 2H), |
| | (M − H)− | 3.29-3.17 (m, 2H), 3.03-2.80 (m, 4H), 1.86 (br d, J = 10.0 Hz, 2H), |
| | | 1.43-1.32 (m, 3H), 1.30-1.20 (m, 2H), 0.87 (t, J = 7.4 Hz, 3H) |
| 126 | LCMS (ESI−): | ¹H NMR (400 MHz, CD₃OD) δ 6.58-6.71 (m, 2 H) 4.24 (s, 2 H) 4.16 (br |
| | m/z = 356.0 | t, J = 9.54 Hz, 2 H) 3.74-3.86 (m, 2 H) 3.39-3.47 (m, 2 H) 2.69-2.89 (m, |
| | (M − H)− | 3 H) 1.58-1.73 (m, 2 H) 0.90 (t, J = 7.40 Hz, 3 H) |
| 127 | LCMS (ESI−): | ¹H NMR (400 MHz, DMSO-d₆) δ 6.53-6.71 (m, 2 H) 3.88-3.97 (m, 2 H) |
| | m/z = 356.0 | 3.73-3.86 (m, 3 H) 3.35-3.47 (m, 2 H) 2.63-2.72 (m, 3 H) 1.17-1.38 (m, 6 H) |
| | (M − H)− | |
| 128 | LCMS (ESI−): | ¹H NMR (400 MHz, DMSO-d₆) δ 10.31-10.89 (m, 1 H) 9.43 (s, 1 H) |
| | m/z = 390.0 | 7.31-7.50 (m, 4 H) 6.60-6.77 (m, 2 H) 4.42-4.79 (m, 4 H) 3.94 (s, 2 H) |
| | (M − H)− | 3.54 (br d, J = 6.72 Hz, 2 H) 2.83-2.98 (m, 2 H) |
| 129 | LCMS (ESI−): | ¹H NMR (400 MHz, CD₃OD) δ 6.66 (m, 2H), 4.23 (s, 2H), 4.04-3.99 |
| | m/z = 384.1 | (m, 4H), 3.39 (m, 2H), 2.82 (t, J = 7.60 Hz), 0.94 (s, 9H). |
| | (M − H)− | |
| 130 | LCMS (ESI−): | ¹H NMR (400 MHz, DMSO-d6) δ 9.62 (br, 1H), 9.41 (s, 1H), 6.68-6.62 |
| | m/z = 424.0 | (m, 2H), 3.93 (s, 2H), 3.71-3.24 (m, 6H), 2.87-2.83 (m, 3H), 2.50 (m, |
| | (M − H)− | 2H), 2.15-2.14 (m, 1H), 1.82-1.63 (m, 1H) |
| 131 | LCMS (ESI−): | ¹H NMR (400 MHz, CD₃OD) δ 6.71-6.68 (m, 2H), 4.39 (s, 2H), 3.82 |
| | m/z = 368.1 | (br, 1H), 3.45-3.31 (m, 5H), 3.01 (t, J = 8.0 Hz, 2 H), 2.18 (m, 1H), 1.99 |
| | (M − H)⁻ | (m, 1H), 0.83-0.69 (m, 4H). |
| 132 | LCMS (ESI−): | ¹H NMR (400 MHz, D₂O) δ 6.68-6.67 (m, 2H), 4.31 (s. 2H), 4.13 (t, J = |
| | m/z = 370.1 | 8.8 Hz, 2H), 3.95 (m, 1H), 3.72 (t, J = 8.8 Hz, 2H), 3.45 (m, 2H), 2.83 |
| | (M − H)⁻ | (m, 2H), 2.47 (m, 1H), 1.74 (m, 1H), 0.77 (m, 6H). |
| 133 | LCMS (ESI−): | ¹H NMR (400 MHz, D₂O) δ 6.73-6.69 (m, 2H), 4.32 (s, 2H), 4.32 (m, |
| | m/z = 368.1 | 2H), 3.41-3.32 (m, 3H), 3.01-2.92 (m, 3H), 2.69 (m, 1H), 1.96 (m, 1H), |
| | (M − H)⁻ | 1.77-1.67 (m, 4H), 1.41 (m, 1H). |
| 134 | LCMS (ESI−): | ¹H NMR (400 MHz, CD₃OD) δ 6.71-6.69 (m, 2H), 4.37 (s, 2H), 3.76- |
| | m/z = 384.1 | 3.73 (m, 1H), 3.45-3.42 (m, 2H), 3.31-3.10 (m, 2H), 3.01-2.82 (m, 3H), |
| | (M − H)⁻ | 2.24-2.03 (m, 2H), 1.68 (m, 1H), 1.59 (m, 1H), 1.00 (t, J = 6.8 Hz, 6H). |
| 135 | LCMS (ESI−): | ¹H NMR (400 MHz, D₂O) δ 6.75-6.73 (m, 2H), 4.35 (s, 2H), 3.70-3.68 |
| | m/z = 384.1 | (m, 1H), 3.47-3.43 (m, 2H), 3.31-2.97 (m, 4H), 2.77 (m, 1H), 2.20-1.91 |
| | (M − H)⁻ | (m, 2H), 1.60-1.40 (m, 2H), 0.90 (t, J = 6.8 Hz, 6H). |
| 136 | LCMS (ESI+): | ¹H NMR (400 MHz, D₂O) δ 6.72-6.70 (m, 2H), 5.42-5.27 (m, 1H), 4.42- |
| | m/z = 348.0 | 4.37 (m, 2H), 4.34 (s, 2H), 4.25-4.20 (m, 2H), 3.55 (t, J = 7.2 Hz, 2H), |
| | (M − H)⁺ | 2.90 (t, J = 7.2 Hz, 2H). |
| 137 | LCMS (ESI−): | ¹H NMR (400 MHz, D₂O) δ 6.72-6.70 (m, 2H), 4.34 (s, 2H), 4.25-4.19 |
| | m/z = 396.0 | (m, 4H), 3.72 (sextuplet, J = 8.0 Hz, 1H), 3.50 (t, J = 7.2 Hz, 2H), 2.87 |
| | (M − H)⁻ | (t, J = 7.2 Hz, 2H). |
| 138 | LCMS (ESI−): | ¹H NMR (400 MHz, D₂O) δ 6.73-6.71 (m, 2H), 4.34 (s, 2H), 3.53-3.37 |
| | m/z = 356.0 | (br, 3H). 3.46 (t, J = 8.0 Hz, 2H), 2.99 (t, J = 8.0 Hz, 2H), 2.77 (br, 1H), |
| | (M − H)⁻ | 2.40 (br, 1H), 2.20 (br, 1H), 1.64 (br, 1H), 1.07 (d, J = 6.8 Hz, 3H). |
| 139 | LCMS (ESI−): | ¹H NMR (400 MHz, D₂O) δ 6.69-6.67 (m, 2H), 4.31 (s, 2H), 3.55-3.25 |
| | m/z = 356.0 | (br, 3H). 3.42 (t, J = 8.0 Hz, 2H), 2.95 (t, J = 8.0 Hz, 2H), 2.75 (br, 1H), |
| | (M − H)⁻ | 2.40 (br, 1H), 2.16 (br, 1H), 1.60 (br, 1H), 1.03 (d, J = 6.8 Hz, 3H). |
| 140 | LCMS (ESI−): | ¹H NMR (400 MHz, DMSO-d₆) δ 9.41 (s, 1H), 6.64-6.59 (m, 2H), 4.09 |
| | m/z = 342.0 | (t, J = 8.0 Hz, 2H), 3.93 (s, 2H), 3.68 (m, 2H), 3.36 (m, 2H), 2.82 |
| | (M − H)⁻ | (sextuplet, J = 7.6 Hz, 1H), 2.69 (t, J = 8.0 Hz, 2H). 1.17 (d, J = 6.4 Hz, 3H). |
| 141 | LCMS (ESI−): | ¹H NMR (400 MHz, D₂O) δ 6.71-6.68 (m, 2H), 4.33 (s, 2H), 4.23 (m, |
| | m/z = 410.0 | 2H), 3.97 (m, 2H), 3.51 (m, 2H), 3.21 (m, 1H), 2.87 (t, J = 6.8 Hz, 2H), |
| | (M − H)⁻ | 2.52 (m, 2H). |
| 142 | LCMS (ESI+): | ¹H NMR (400 MHz, D₂O) δ 6.74-6.68 (m, 2H), 4.58 (m, 1H), 4.47 (m, |
| | m/z = 362.0 | 1H), 4.34 (s, 2H), 4.18 (m, 2H), 4.06 (m, 2H), 3.48 (t, J = 6.8 Hz, 2H), |
| | (M − H)⁺ | 3.19-3.11 (m, 1H), 2.87 (t, J = 6.8 Hz, 2H). |

### Example A: Enzymatic Assay Used to Determine Potency of PTPN2 Inhibitors

Compound activity was determined in an in vitro enzymatic assay using untagged, full-length human PTPN2 (TC45) (1-387) protein. PTPN2 was produced in E. coli as a GST-TEV fusion and the GST was removed by TEV digestion, followed by additional purification to yield full-length PTPN2 (SEQ ID 1). PTPN2 enzyme was diluted in assay buffer (50 mM HEPES pH7.5, 0.2 mM EDTA, 1 mM DTT, 0.02% Brij-35, 0.02% BSA) to a final concentration of 0.5 nM and added to black 384-well non-binding plates (Greiner, 781900). Compounds were subsequently added using a Tecan D300e dispenser. Following a 10 min incubation at room temperature, DiFMUP substrate (ThermoFisher, D22065) was added to a final concentration of 100 μM. Plates were transferred to a SpectraMax plate reader (Molecular Devices) and fluorescence intensity was measured (ex 358, em 455) after a 30 min incubation at room temperature. Each plate included a 100% inhibition control (no enzyme) and a 0% inhibition control (DMSO) from which % inhibition for test compounds was calculated. A four-parameter curve fit was used to determine IC₅₀ values from % inhibition data.

### Example B: B16F10 Cellular Growth Inhibition Assay

Compound activity was determined using an interferon gamma (IFNγ)-induced cellular growth inhibition assay with the murine B16F10 melanoma cell line on an Agilent xCELLigence Real-Time Cell Analysis platform (RTCA). RTCA E-Plate View 96 plates (Agilent, 300601010) were pre-equilibrated with 50 μL of assay media (DMEM+10% FBS, Gibco 10566-024, Gibco 10082-147) at 37° C. in a humidified incubator before taking an initial measurement of impedance (sweep). B16F10 cells cultured in assay media were dissociated with TrypLE Express (Gibco 12605-010) for five minutes at 37° C., diluted in 3 volumes of assay buffer, centrifuged for 5 minutes at 500×g at room temperature before diluting cells to 7,700 cells/mL in assay media, plating 130 μL/well (1,000 cells/well) in the inner 60 wells of the assay plate, and adding 150 μL of assay media to the outer wells of the plate. Cells were incubated at room temperature for 20 min to allow cells to settle before placing them in the xCELLigence reader and incubating overnight at 37° C., sweeping wells every 15 minutes. After 24 hours, well readings were paused, plates were removed from the incubator and compounds were added using a Tecan D300e dispenser. All wells were normalized to a final concentration of 0.5% DMSO. Following a 30 min incubation at 37° C., recombinant mouse IFNγ (R&D Systems™ 485MI100) was diluted to 10 ng/mL in assay media and 20 μL was added to assay wells (1 ng/mL final concentration). Assay plates were placed in the xCELLigence reader and swept every 15 minutes. After 48 hours, well readings were normalized to the time point immediately preceding compound addition and the area under the growth curve (AUC) was calculated by the RTCA software and exported. A four-parameter curve fit was used to determine compound IC₅₀ values using % inhibition for each compound concentration calculated using the DMSO vehicle with IFNγ treatment as baseline (0% inhibition) and a positive control PTPN2 inhibitor with IFNγ treatment as 100% inhibition.

The data from Example A and Example B is shown in table 3.

TABLE 3

| | Full Length PTPN2 | B16 IFNγ Mediated Growth |
|---|---|---|
| | DiFMUP Assay: AVG | Inhibition Assay: AVG |
| Ex | IC₅₀ (μM) | IC₅₀ (μM) |
| 1 | 0.007 | NT |
| 2 | 0.014 | 28.2 |
| 3 | 0.003 | >100 |
| 4 | 0.014 | >100 |
| 5 | 0.007 | 84.6 |
| 6 | 0.020 | >100 |
| 7 | 0.007 | 2.47 |
| 8 | 0.088 | 18.6 |
| 9 | 0.021 | 13.2 |
| 10 | 0.050 | 17.4 |
| 11 | 0.018 | 5.18 |
| 12 | 0.012 | 4.34 |
| 13 | 0.013 | 36.2 |
| 14 | 0.014 | 52.8 |
| 15 | 0.024 | >48 |
| 16 | 0.016 | 35.8 |
| 17 | 0.007 | >52 |
| 18 | 0.007 | >58 |
| 19 | 0.012 | 8.43 |
| 20 | 0.015 | 2.62 |
| 21 | 0.015 | 4.59 |
| 22 | 0.010 | 1.23 |
| 23 | 0.014 | 4.33 |
| 24 | 0.015 | 5.90 |
| 25 | 0.015 | NT |
| 26 | 0.008 | NT |
| 27 | 0.007 | 9.73 |
| 28 | 0.023 | >57 |
| 29 | 0.013 | >62 |
| 30 | 0.013 | >13 |
| 31 | 0.047 | 29.7 |
| 32 | 0.035 | 14.8 |
| 33 | 0.035 | 28.8 |
| 34 | 0.043 | 9.96 |
| 35 | 0.031 | 13.0 |
| 36 | 0.022 | 81.0 |
| 37 | 0.066 | 46.4 |
| 38 | 0.154 | >100 |
| 39 | 0.063 | >100 |
| 40 | 0.046 | 38.6 |
| 41 | 0.047 | >100 |
| 42 | 0.012 | >100 |
| 43 | 0.063 | 95.9 |
| 44 | 0.004 | 38.0 |
| 45 | 0.030 | 55.4 |
| 46 | 0.075 | >100 |
| 47 | 0.098 | NT |
| 48 | 0.223 | >33 |
| 49 | 0.096 | >38 |
| 50 | 0.091 | >100 |
| 51 | 0.063 | 27.6 |
| 52 | 0.044 | >73 |
| 53 | 0.035 | >100 |
| 54 | 0.075 | 25.8 |
| 55 | 0.016 | >100 |
| 56 | 0.028 | >45 |
| 57 | 0.015 | NT |
| 58 | 0.051 | >100 |
| 59 | 0.016 | >47 |
| 60 | 0.024 | >73 |
| 61 | 0.019 | >41 |
| 62 | 0.016 | >100 |
| 63 | 0.075 | >100 |
| 64 | 0.089 | >100 |
| 65 | 0.056 | 41.3 |
| 66 | 0.016 | 4.27 |
| 67 | 0.036 | >100 |
| 68 | 0.046 | >100 |
| 69 | 0.015 | 13.6 |
| 70 | 0.204 | >45 |
| 71 | 0.065 | 19.3 |
| 72 | 0.066 | 8.44 |
| 73 | 0.092 | 8.62 |
| 74 | 0.161 | 9.29 |
| 75 | 0.020 | NT |
| 76 | 0.006 | 8.92 |
| 77 | 0.004 | >100 |
| 78 | 0.013 | 11 |
| 79 | 0.007 | 3.45 |
| 80 | 0.005 | 22.7 |
| 81 | 0.003 | 0.363 |
| 82 | 0.076 | 36.3 |
| 83 | 0.003 | 0.674 |
| 84 | 0.013 | 13.7 |
| 85 | 0.002 | 0.331 |
| 86 | 0.004 | 0.651 |
| 87 | 0.003 | 0.299 |
| 88 | 0.003 | 1.02 |
| 89 | 0.007 | 8.91 |
| 90 | 0.002 | 1.22 |
| 91 | 0.036 | >24 |
| 92 | 0.003 | 0.504 |
| 93 | 0.003 | 0.475 |
| 94 | 0.002 | 0.469 |
| 95 | 0.002 | 0.432 |
| 96 | 0.002 | 0.680 |
| 97 | 0.003 | 1.54 |
| 98 | 0.005 | 1.05 |
| 99 | 0.002 | 0.183 |
| 100 | 0.005 | 51.0 |
| 101 | 0.002 | 1.69 |
| 102 | 0.002 | 0.605 |
| 103 | 0.050 | 13.2 |
| 104 | 0.003 | 0.723 |
| 105 | 0.003 | 0.219 |
| 106 | 0.002 | 0.256 |
| 107 | 0.002 | 0.256 |
| 108 | 0.002 | 0.129 |
| 109 | 0.003 | 0.162 |
| 110 | 0.002 | 0.127 |
| 111 | 0.002 | 0.130 |
| 112 | 0.002 | 0.165 |
| 113 | 0.002 | 0.216 |
| 114 | 0.007 | 0.371 |
| 116 | 0.002 | 0.172 |
| 117 | 0.001 | 0.331 |
| 118 | 0.003 | 3.22 |
| 119 | 0.002 | 2.43 |
| 119 | 0.003 | 1.60 |
| 120 | 0.002 | 2.64 |
| 121 | 0.002 | 2.83 |
| 122 | 0.003 | 2.67 |
| 123 | 0.002 | 1.84 |
| 124 | 0.002 | 0.943 |
| 125 | 0.002 | 3.09 |
| 126 | 0.003 | 0.775 |
| 127 | 0.002 | 0.657 |
| 128 | 0.002 | 0.610 |
| 129 | 0.002 | 0.660 |
| 130 | 0.001 | 0.208 |
| 131 | 0.001 | 0.489 |
| 132 | 0.003 | 0.579 |
| 133 | 0.003 | 1.04 |
| 134 | 0.001 | 0.516 |
| 135 | 0.002 | 0.290 |
| 136 | 0.003 | 0.937 |
| 137 | 0.005 | 2.16 |
| 138 | 0.002 | 0.406 |
| 139 | 0.002 | 0.509 |
| 140 | 0.002 | 0.472 |
| 141 | 0.003 | 0.370 |
| 142 | 0.003 | 0.542 |

NT: not tested

What is claimed is:

1. A compound of Formula (I), or a pharmaceutically acceptable salt, solvate, or stereoisomer thereof: 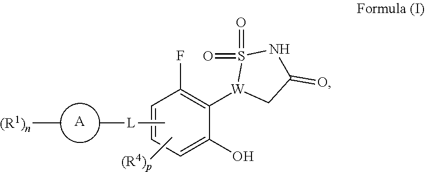
wherein:
Ring A is a heterocycloalkyl or heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{e}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R^{1a};
or two R¹ on the same atom are taken together to form an oxo;
or two R¹ on the same carbon are taken together to form a cycloalkyl or heterocycloalkyl; each optionally substituted with one or more R;
or two R¹ on the different atoms are taken together to form a cycloalkyl, heterocycloalkyl, aryl, or heteroaryl; each optionally substituted with one or more R;
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{a}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R;
or two R^{1a} on the same atom are taken together to form an oxo;
n is 0-11;
L is —O—, —S—, —S(═O)—, —S(═O)₂—, —NR²—, [C(R³)₂]ₘ, —O[C(R³)₂]ₘ—, —NR²[C(R³)₂]ₘ, —[C(R³)₂]ₘO—, or —[C(R³)₂]ₘNR²—;
R² is hydrogen, —C(═O)R^{a}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
each R³ is independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R;
or two R³ are taken together to form a cycloalkyl or heterocycloalkyl; each optionally substituted with one or more R;
m is 1-4;
each R⁴ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
p is 0-2;
W is CR^{W} or N;
R^{W} is hydrogen, deuterium, halogen, —CN, —NO₂, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{a}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently and optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently and optionally substituted with one or more R;
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently and optionally substituted with one or more R;
or R^{c} and R^{d} are taken together with the atom to which they are attached to form a heterocycloalkyl optionally substituted with one or more R; and
each R is independently halogen, —CN, —OH, —OC₁-C₃alkyl, —OC₁-C₃haloalkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃deuteroalkyl, C₁-C₃haloalkyl, C₁-C₃hydroxyalkyl, C₁-C₃aminoalkyl, C₁-C₃heteroalkyl, or C₃-C₆cycloalkyl;
or two R on the same atom form an oxo.

2. The compound of claim 1, or a pharmaceutically acceptable salt, solvate, or stereoisomer thereof,
wherein the compound is of Formula (Ia): 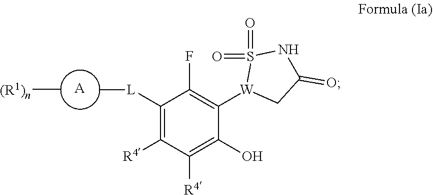
wherein R^{4′} is hydrogen or R⁴.

3. The compound of claim 1, or a pharmaceutically acceptable salt, solvate, or stereoisomer thereof,
wherein the compound is of Formula (Ib): 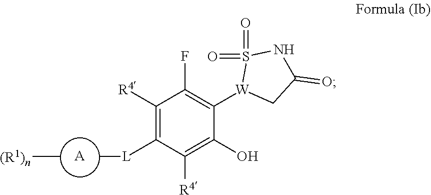
wherein R^{4′} is hydrogen or R⁴.

4. The compound of claim 1, or a pharmaceutically acceptable salt, solvate, or stereoisomer thereof,
wherein the compound is of Formula (Ic): 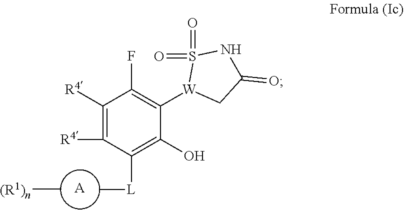
wherein R^{4′} is hydrogen or R⁴.

5. The compound of any one of claims 1-4, or a pharmaceutically acceptable salt, solvate, or stereoisomer thereof, wherein Ring A is heterocycloalkyl.

6. The compound of any one of claims 1-4, or a pharmaceutically acceptable salt, solvate, or stereoisomer thereof; wherein Ring A is monocyclic heterocycloalkyl.

7. The compound of any one of claims 1-4, or a pharmaceutically acceptable salt, solvate, or stereoisomer thereof, wherein Ring A is bicyclic heterocycloalkyl.

8. The compound of any one of claims 1-4, or a pharmaceutically acceptable salt, solvate, or stereoisomer thereof, wherein Ring A is 4- to 8-membered heterocycloalkyl.

9. The compound of any one of claims 1-4, or a pharmaceutically acceptable salt, solvate, or stereoisomer thereof, wherein Ring A is 5- to 8-membered heterocycloalkyl.

10. The compound of any one of claims 1-4, or a pharmaceutically acceptable salt, solvate, or stereoisomer thereof, wherein Ring A is 5- to 6-membered heterocycloalkyl.

11. The compound of any one of claims 5-10, or a pharmaceutically acceptable salt, solvate, or stereoisomer thereof; wherein the heterocycloalkyl comprises 1 to 4 heteroatoms selected from the group consisting of O, S, and N.

12. The compound of any one of claims 5-10, or a pharmaceutically acceptable salt, solvate, or stereoisomer thereof; wherein the heterocycloalkyl comprises 1 to 3 heteroatoms selected from the group consisting of O, S, and N.

13. The compound of any one of claims 5-10, or a pharmaceutically acceptable salt, solvate, or stereoisomer thereof; wherein the heterocycloalkyl comprises 1 to 3 heteroatoms selected from the group consisting of O and N.

14. The compound of any one of claims 5-10, or a pharmaceutically acceptable salt, solvate, or stereoisomer thereof; wherein the heterocycloalkyl comprises 1 to 2 heteroatoms selected from the group consisting of O and N.

15. The compound of any one of claims 5-10, or a pharmaceutically acceptable salt, solvate, or stereoisomer thereof, wherein the heterocycloalkyl comprises 1 to 2 heteroatoms that are N.

16. The compound of any one of claims 1-4, or a pharmaceutically acceptable salt, solvate, or stereoisomer thereof, wherein Ring A is azetidinyl, pyrrolidinyl, piperidinyl, piperazinyl, morpholinyl, or azepanyl.

17. The compound of any one of claims 1-4, or a pharmaceutically acceptable salt, solvate, or stereoisomer thereof, wherein Ring A is pyrrolidinyl or piperidinyl.

18. The compound of any one of claims 1-4, or a pharmaceutically acceptable salt, solvate, or stereoisomer thereof, wherein Ring A is pyrrolidinyl.

19. The compound of any one of claims 1-4, or a pharmaceutically acceptable salt, solvate, or stereoisomer thereof; wherein Ring A is piperidinyl.

20. The compound of any one of claims 1-4, or a pharmaceutically acceptable salt, solvate, or stereoisomer thereof, wherein Ring A is 6-azaspiro[3.4]octanyl, 7-azaspiro[3.5]nonanyl, 6-azaspiro[2.5]octanyl, 2-azaspiro[4.4]nonanyl, 8-oxa-2-azaspiro[4.5]decanyl, 2-azaspiro[3.4]octanyl, 2-oxa-7-azaspiro[4.4]nonanyl, 2-azaspiro[4.5]decanyl, or 2-azaspiro[3.3]heptanyl.

21. The compound of any one of claims 1-20, or a pharmaceutically acceptable salt, solvate, or stereoisomer thereof; wherein L is —[C(R³)₂]ₘ—.

22. The compound of any one of claims 1-21, or a pharmaceutically acceptable salt, solvate, or stereoisomer thereof, wherein each R³ is independently hydrogen, deuterium, halogen, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; or two R³ are taken together to form a cycloalkyl.

23. The compound of any one of claims 1-21, or a pharmaceutically acceptable salt, solvate, or stereoisomer thereof, wherein each R³ is independently hydrogen, deuterium, halogen, —OH, C₁-C₆alkyl, or C₁-C₆haloalkyl; or two R³ are taken together to form a cycloalkyl.

24. The compound of any one of claims 1-21, or a pharmaceutically acceptable salt, solvate, or stereoisomer thereof, wherein each R³ is independently hydrogen, halogen, —OH, or C₁-C₆alkyl; or two R³ are taken together to form a cycloalkyl.

25. The compound of any one of claims 1-21, or a pharmaceutically acceptable salt, solvate, or stereoisomer thereof, wherein each R³ is independently hydrogen or C₁-C₆alkyl.

26. The compound of any one of claims 1-21, or a pharmaceutically acceptable salt, solvate, or stereoisomer thereof, wherein each R³ is hydrogen.

27. The compound of any one of claims 1-26, or a pharmaceutically acceptable salt, solvate, or stereoisomer thereof; wherein m is 1 or 2.

28. The compound of any one of claims 1-27, or a pharmaceutically acceptable salt, solvate, or stereoisomer thereof; wherein m is 1.

29. The compound of any one of claims 1-27, or a pharmaceutically acceptable salt, solvate, or stereoisomer thereof; wherein m is 2.

30. The compound of any one of claims 1-26, or a pharmaceutically acceptable salt, solvate, or stereoisomer thereof, wherein L is —CH₂—, —CH₂CH₂—, or —CH₂CH₂CH₂—.

31. The compound of any one of claims 1-26, or a pharmaceutically acceptable salt, solvate, or stereoisomer thereof; wherein L is —CH₂—.

32. The compound of any one of claims 1-26, or a pharmaceutically acceptable salt, solvate, or stereoisomer thereof; wherein L is —CH₂CH₂—.

33. The compound of any one of claims 1-26, or a pharmaceutically acceptable salt, solvate, or stereoisomer thereof; wherein L is —CH₂CH₂CH₂—.

34. The compound of any one of claims 1-33, or a pharmaceutically acceptable salt, solvate, or stereoisomer thereof, wherein each R⁴ is independently deuterium, halogen, C₁-C₆alkyl, or C₁-C₆haloalkyl.

35. The compound of any one of claims 1-34, or a pharmaceutically acceptable salt, solvate, or stereoisomer thereof, wherein each R⁴ is independently deuterium or halogen.

36. The compound of any one of claims 1-35, or a pharmaceutically acceptable salt, solvate, or stereoisomer thereof; wherein p is 0 or 1.

37. The compound of any one of claims 1-36, or a pharmaceutically acceptable salt, solvate, or stereoisomer thereof; wherein p is 0.

38. The compound of any one of claims 1-36, or a pharmaceutically acceptable salt, solvate, or stereoisomer thereof; wherein p is 1.

39. The compound of any one of claims 1-38, or a pharmaceutically acceptable salt, solvate, or stereoisomer thereof; wherein W is N.

40. The compound of any one of claims 1-39, or a pharmaceutically acceptable salt, solvate, or stereoisomer thereof, wherein each R¹ is independently deuterium, halogen, —CN, —OH, —OR^{a}, —NR^{c}R^{d}, —C(═O)R^{a}, —C(═O)OR^{a}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R¹.

41. The compound of any one of claims 1-39, or a pharmaceutically acceptable salt, solvate, or stereoisomer thereof, wherein each R¹ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R¹.

42. The compound of any one of claims 1-39, or a pharmaceutically acceptable salt, solvate, or stereoisomer thereof, wherein each R¹ is independently deuterium, halogen, —OH, —OR^{a}, C₁-C₆alkyl, C₁-C₆haloalkyl, C₁-C₆hydroxyalkyl, C₁-C₆heteroalkyl, or cycloalkyl; wherein each alkyl and cycloalkyl is independently and optionally substituted with one or more R¹.

43. The compound of any one of claims 1-39, or a pharmaceutically acceptable salt, solvate, or stereoisomer thereof, wherein each R¹ is independently halogen, —OH, —OR^{a}, C₁-C₆alkyl, C₁-C₆haloalkyl, C₁-C₆hydroxyalkyl, C₁-C₆heteroalkyl, or cycloalkyl.

44. The compound of any one of claims 1-39, or a pharmaceutically acceptable salt, solvate, or stereoisomer thereof, wherein each R¹ is independently halogen, C₁-C₆alkyl, or C₁-C₆haloalkyl.

45. The compound of any one of claims 1-39, or a pharmaceutically acceptable salt, solvate, or stereoisomer thereof, wherein each R¹ is independently halogen or C₁-C₆alkyl.

46. The compound of any one of claims 1-39, or a pharmaceutically acceptable salt, solvate, or stereoisomer thereof, wherein each R¹ is independently C₁-C₆alkyl.

47. The compound of any one of claims 1-39, or a pharmaceutically acceptable salt, solvate, or stereoisomer thereof, wherein each R¹ is independently C₁-C₆alkyl optionally substituted with one or more R^{1a}.

48. The compound of any one of claims 1-39, or a pharmaceutically acceptable salt, solvate, or stereoisomer thereof, wherein each R¹ is independently C₁-C₆alkyl.

49. The compound of any one of claims 1-48, or a pharmaceutically acceptable salt, solvate, or stereoisomer thereof, wherein each R^{1a} is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R.

50. The compound of any one of claims 1-48, or a pharmaceutically acceptable salt, solvate, or stereoisomer thereof, wherein each R^{1a} is independently deuterium, halogen, —CN, —OH, —OR^{a}, —NR^{c}R^{d}, C₁-C₆alkyl, C₁-C₆haloalkyl, cycloalkyl, heterocycloalkyl, aryl, or heteroaryl; wherein each alkyl, cycloalkyl, heterocycloalkyl, aryl, and heteroaryl is independently and optionally substituted with one or more R.

51. The compound of any one of claims 1-48, or a pharmaceutically acceptable salt, solvate, or stereoisomer thereof, wherein each R^{1a} is independently deuterium, halogen, —CN, —OH, —OR^{a}, —NR^{c}R^{d}, C₁-C₆alkyl, C₁-C₆haloalkyl, cycloalkyl, heterocycloalkyl, aryl, or heteroaryl.

52. The compound of any one of claims 1-48, or a pharmaceutically acceptable salt, solvate, or stereoisomer thereof, wherein each R^{1a} is independently halogen, —NR^{c}R^{d}, C₁-C₆alkyl, C₁-C₆haloalkyl, cycloalkyl, heterocycloalkyl, aryl, or heteroaryl.

53. The compound of any one of claims 1-48, or a pharmaceutically acceptable salt, solvate, or stereoisomer thereof, wherein each R^{1a} is independently —NR^{c}R^{d}, cycloalkyl, heterocycloalkyl, aryl, or heteroaryl.

54. The compound of any one of claims 1-48, or a pharmaceutically acceptable salt, solvate, or stereoisomer thereof, wherein each R^{1a} is independently cycloalkyl, heterocycloalkyl, aryl, or heteroaryl.

55. The compound of any one of claims 1-54, or a pharmaceutically acceptable salt, solvate, or stereoisomer thereof; wherein n is 0-2.

56. The compound of any one of claims 1-54, or a pharmaceutically acceptable salt, solvate, or stereoisomer thereof; wherein n is 0 or 1.

57. The compound of any one of claims 1-54, or a pharmaceutically acceptable salt, solvate, or stereoisomer thereof; wherein n is 0.

58. The compound of any one of claims 1-54, or a pharmaceutically acceptable salt, solvate, or stereoisomer thereof; wherein n is 1.

59. The compound of claim 1, or a pharmaceutically acceptable salt, solvate, or stereoisomer thereof, wherein the compound is selected from a compound of table 1 or 2.

60. A pharmaceutical composition comprising a compound of any one of claims 1-59, or a pharmaceutically acceptable salt, solvate, or stereoisomer thereof, and a pharmaceutically acceptable excipient.

61. A method of treating cancer in a subject in need thereof, the method comprising administering to the subject an effective amount of a compound of any on of claim 1-59, or a pharmaceutically acceptable salt, solvate, or stereoisomer thereof.

62. A method of treating cancer in a subject in need thereof, the method comprising administering to the subject a pharmaceutical composition of claim 60.

63. The method of claim 61 or 62, further comprising administering an additional therapeutic agent.

64. The method of claim 63, wherein the additional therapeutic agent is an immunotherapeutic agent.

65. The method of claim 64, wherein the immunotherapeutic agent is an anti-PD-1 antibody, an anti-PD-L1 antibody, or an anti-CTLA-4 antibody.

66. A method of treating type-2 diabetes in a subject in need thereof, the method comprising administering to the subject an effective amount of a compound of any one of claims 1-59, or a pharmaceutically acceptable salt, solvate, or stereoisomer thereof.

67. A method of treating type-2 diabetes in a subject in need thereof, the method comprising administering to the subject a pharmaceutical composition of claim 60.

68. A method of treating and/or controlling obesity in a subject in need thereof, the method comprising administering to the subject an effective amount of a compound of any one of claims 1-59, or a pharmaceutically acceptable salt, solvate, or stereoisomer thereof.

69. A method of treating and/or controlling obesity in a subject in need thereof, the method comprising administering to the subject a pharmaceutical composition of claim 60.

70. A method of treating a metabolic disease in a subject in need thereof, the method comprising administering to the subject an effective amount of a compound of any one of claims 1-59, or a pharmaceutically acceptable salt, solvate, or stereoisomer thereof.

71. A method of treating a metabolic disease in a subject in need thereof, the method comprising administering to the subject a pharmaceutical composition of claim 60.
